SEQUENCE LISTING (72 sequences)

SEQ ID NO: 1 - DNA, 1723 bp, Human cyclovirus
atacccgccacttcgttgcacgatggcaaaccgtacagtgcgccgattctgcttcacgtggaacgaccatccagtcgaagcatacgaaaagtgcgaaaagtttattgaaaaattctgcaaatatggaatcgtgggagaagaatacgctccaactacaggaatgccccatctccaaggtttctgtaaccttaataaaccaacacgcttcagtaccatcaagaagcacctcgataacagcatccatattgagaaggcaaatgggacagacgaacaaaaccaaaaatactgttcaaaatcaggcatattttttgaatcgggcgtacctaacaaacaagggcaacgcaatgatctgcaatctctggtggaattcattcacgaaaagaggccaacgatacgagatattgccaccgaacatcctacgacatacatacgctacttccgtggaatcgaaagaatgctgcaactggttaacccgatcaaacaacgcgatttcaagactgaagtatattattattggggaccgccggggactggcaaatctagaagagcacttgaagaggcacaagcattcaacactgagtcaatatactataaaccacgcggattgtggtgggacggatacgaacaacaggacagcgtcatcatagacgacttctacggatggatcaaatacgacgaaatgctgaaaataatggaccgctacccatacaaggtacaagtgaaaggagcttttcaagaatttacaagcaaaaaaatttttattacatcaaatgttgatacagacgaattatacaaatttgtaggctatacaacagccgcttttgaacgtcgcattacaaataaagaatacatggcttaaataaatgagcgttgatttttatatttaacaaacaaatccataacaactgtaaaattaacaggatgcgattccgcaatagtactatctccctcaaaacataaaaatccacaatataatatttccttatccgcatcaactcctatatcaaactcaggtcgccattttgtagtattatataaagtagaggtctccgtatcagtaccaagacgaacagcaggcacaaaactcatacgtccaaattgcgtcatccttcgtatcttgctcttatcaatagataaacaggtcggaaagttcggagtagcaggtttcacaagaggtcgatgatatggtacaatagcataattagtaactcgcgatgacgtattattcgcaacgttctgttgtggaaaaacccttacattcagtctatatatcttcacacgttcaaaatttggtgcgaggttcttatgttcagaaaattgttccaacgtttgatgcagggaagccacaccaccagttcttgcatcagaatatacttgcacagaacgagtaagtttagtaaataatgtagaaattggacggcggcgaaatcgcagtctacgcttgaaaactcgcctgcgagaccggacaaaccggttcctacgctggggcattcgccgcaaacgcttcaaggccatgcctttcactttaacgctcggtataaaaataactcctcttcgggtgacggcttgttgccaagtgaacgttgcgcccccccccgtcggccgggcaggctccccggcggggggagggcgtgcgtgttggaacgtgttacgggcggatgttagcggagcggatagtacatgcggttacgaagtcacggttacgaagtggcggggtaatact

SEQ ID NO: 2 - protein, 278 aa, Human cyclovirus
MANRTVRRFCFTWNDHPVEAYEKCEKFIEKFCKYGIVGEEYAPTTGMPHLQGFCNLNKPTRFSTIKKHLDNSIHIEKANGTDEQNQKYCSKSGIFFESGVPNKQGQRNDLQSLVEFIHEKRPTIRDIATEHPTTYIRYFRGIERMLQLVNPIKQRDFKTEVYYYWGPPGTGKSRRALEEAQAFNTESIYYKPRGLWWDGYEQQDSVIIDDFYGWIKYDEMLKIMDRYPYKVQVKGAFQEFTSKKIFITSNVDTDELYKFVGYTTAAFERRITNKEYMA

SEQ ID NO: 3 - protein, 219 aa, Human cyclovirus
MALKRLRRMPQRRNRFVRSRRRVFKRRLRFRRRPISTLFTKLTRSVQVYSDARTGGVASLHQTLEQFSEHKNLAPNFERVKIYRLNVRVFPQQNVANNTSSRVTNYAIVPYHRPLVKPATPNFPTCLSIDKSKIRRMTQFGRMSFVPAVRLGTDTETSTLYNTTKWRPEFDIGVDADKEILYCGFLCFEGDSTIAESHPVNFTVVMDLFVKYKNQRSFI

SEQ ID NO: 4 - DNA, 1780 bp, Human cyclovirus
ataccgtccacttcgttgcgccttgttgctgggcgctcccacttatatactctactcgtgcaattcgagagaaactaccgcttggtgttgtcgcccgatcccgctacgcggcggcacctcgcaaacgctgcgctccttcgtcgcgcagcctactcggcacccccgcttcgcgggacgggctctgtgcctcgtagtcgttagccatgaacagtactgtgcgtagattctgtttcacgtggaataactacacggttgcagacactcttactgtgaaagactactttgtgaaatactgcaaatatggcatcgctggagaggaactcgctccggaaaccggaactcctcatcttcagggattctgtaatctacgcaagccccagcgcttcagtgccatcaagaagcatctcagtgacagaatccatattgagaaggcaaatggatccgacgaacaaaaccaatcttactgtaaaaaagcaggccattggtttgaacagggtgtaccagttaaacaaggggacagaagcgaccttaaatctgtcgtttcaaccattgcggatggggccaacaccgcacaagccattgctgaaaaacaccccgttgagtttattaagtactataaagggatcagcgagtatctacgactacttgccccaattgcgccgcgtaaattcaaaacctacgtatactactattggggcccaccaggatctggcaaaagccggcgcgctcttgaagaagcagaatcgctcgacccagattcaatatactacaagccaagagggctatggtgggacggctaccgacagcagcagtctgttatcattgatgacttctacggttggatcaaatacgacgaactcctgaaaataacggaccgctacccatacaaggtgcaagtgaaaggcggcttcgaggaattcacctctacccgcatctggttcacaagcaacgtcgacacggaccggttataccggtttgacgggttcaccccagaggcattcgaaagaagaattacagcgaaaatacatattacttaaacaatcattgtattttgacctttgagtttacaatatacatcaagcttaatactaaaatatgattttgtatctgtagctcccaaattcccttgaaacgcaaccgctccacattgaatgttaggatgttcttgagttccgataagattacatttcaattccggccgccattggacttgtccataagatgtctcattgcttttccaatcggttactagctgggtactcggtacatatacctgccttcctacttgcgtctgacggtacatttttgctcggtccaaagacatatacgcctcaaaatctttaggcatggcgattgatgtgtgccacggcgccattgcgtagcacggacacatcgaggttgaattattagatacattttgcagagggtatacagtagcaacaaccttatagaacttgatatactcaaacgacgttttcaatctctcaaactctttaaaatcattacatttaaatgaaacaggccagacagacgtagatttatgctcaacagtaagaagcgtgaccttcgtaaattttgcgagtaactttcccttcgcgctacgcctatgtctcctgcggtagactcgcctccgccggccaagccgtcgaaccggtttgcgccggtacacccttctccttctgtacgccatgcctgactgcccgtatatggagtgaaaggccgccgcgcgggcgacatggtggggggttacgaagttgttacgaagtggacgggtaatact

SEQ ID NO: 5 - protein, 277 aa, Human cyclovirus
MNSTVRRFCFTWNNYTVADTLTVKDYFVKYCKYGIAGEELAPETGTPHLQGFCNLRKPQRFSAIKKHLSDRIHIEKANGSDEQNQSYCKKAGHWFEQGVPVKQGDRSDLKSVVSTIADGANTAQAIAEKHPVEFIKYYKGISEYLRLLAPIAPRKFKTYVYYYWGPPGSGKSRRALEEAESLDPDSIYYKPRGLWWDGYRQQQSVIIDDFYGWIKYDELLKITDRYPYKVQVKGGFEEFTSTRIWFTSNVDTDRLYRFDGFTPEAFERRITAKIHIT

SEQ ID NO: 6 - protein, 218 aa, Human cyclovirus
MAYRRRRVYRRKPVRRLGRRRRVYRRRHRRSAKGKLLAKFTKVTLLTVEHKSTSVWPVSFKCNDFKEFERLKTSFEYIKFYKVVATVYPLQNVSNNSTSMCPCYAMAPWHTSIAMPKDFEAYMSLDRAKMYRQTQVGRQVYVPSTQLVTDWKSNETSYGQVQWRPELKCNLIGTQEHPNIQCGAVAFQGNLGATDTKSYFSIKLDVYCKLKGQNTMIV

SEQ ID NO: 7 - DNA, 1740 bp, Human cyclovirus
atacccgtcacttcgttgtgaaatggcaaaccgtacagtgcgccgattctgctttacatggaacaactatcctgacacagcgtacgaaaagtgtgaaacattcatacaaaagttctgcaaatatggaatcgtgggagaagaactcgctccaacaacagggacgcctcacctacaagggttttgtaacttacgtaaacccacacgcttcagtaccatcaagaagcatctcgataactcaatccatattgagaaggcaaacggatccgacgaacagaaccaagcatactgttcgaagacaggcatattttttgaagagggtagtcccactaaacaagggcaacgcaacgatttacaacttctcgtggatgatgttatcggacaccctgatattaaaacaaaagatattgccgccaaatatccttctacatttgttagatacttcagaggaattaaagaattacaacgagtggttcatccagtggcagaaagaatgttcaaaactgaggtgtactattattggggacctcctggaactggcaaaagtagaagagccctagaagaagcccaggcgacaggaaacggatcgatatactacaaaccacgcggcctctggtgggacgggtacgaacaacaagattccgttattatagacgacttctacggatggataaaatatgacgaactacttaaaattacagaccgctacccatacaaggtacaaataaaaggaggattcgaagaatttacatcaaaaaaaatttttattacatcaaacgttgatacagatgaactttacaaatttaatggttacaacacagcagctttagaacgccgtttaaccgtcaaggaatatatgtcttaaagcgacgccaatgtattctgtttcttaaatacaacccatacatcatgtttaacattatatctcgctgttccagtcatttcaccattcccttgaaaagctattattccattataaatccttatagctcttgtctgagaattagatatatatacttttggcctaaactgtatcatatcgggtgaggatgtaccacctccggaatctgatagtccctctattaatactgaaggtacatatgtctgcgatccaacttgagtacctctaaatattttagctctatcgattgtcaaataagtattaaacgtagctgatgctggtcctccgcgatgccatggggccatcaaatatgccggtacttgacttgtcgaattattagcaatattctgcatcggatatatagtaactctctctttcaaaaaaacacaatattcaaaattctttttcaactcattgtactccgaaaaatccgtcgggagatatgacaaagaccacacgttaattttagaaatgtcaacattaaccgtatcgattttggtaagtttaaccaagagatcagattttccgacgcgacttttacggcgaaaaaacttgcgacggcgtcgacgataccgccgcagcggtcgccgaacccgtctataacgcctcaaggccatgcctttcactttgacgctcggtataaaataactccacttcaggcggctgcctgttgccaagtgaacgttgcgccccccccctcggccggggagcctgcccggccggtcccccccccgccggccgggcggcccccggcggggggagggcgtgcgtgttgaaacttgtttgggcggttgagtcagaccagttacgaagttacggttacgaagtgacggggtaatact

SEQ ID NO: 8 - protein, 279 aa, Human cyclovirus
MANRTVRRFCFTWNNYPDTAYEKCETFIQKFCKYGIVGEELAPTTGTPHLQGFCNLRKPTRFSTIKKHLDNSIHIEKANGSDEQNQAYCSKTGIFFEEGSPTKQGQRNDLQLLVDDVIGHPDIKTKDIAAKYPSTFVRYFRGIKELQRVVHPVAERMFKTEVYYYWGPPGTGKSRRALEEAQATGNGSIYYKPRGLWWDGYEQQDSVIIDDFYGWIKYDELLKITDRYPYKVQIKGGFEEFTSKKIFITSNVDTDELYKFNGYNTAALERRLTVKEYMS

SEQ ID NO: 9 - protein, 218 aa, Human cyclovirus
MALRRYRRVRRPLRRYRRRRRKFFRRKSRVGKSDLLVKLTKIDTVNVDISKINVWSLSYLPTDFSEYNELKKNFEYCVFLKERVTIYPMQNIANNSTSQVPAYLMAPWHRGGPASATFNTYLTIDRAKIFRGTQVGSQTYVPSVLIEGLSDSGGGTSSPDMIQFRPKVYISNSQTRAIRIYNGIIAFQGNGEMTGTARYNVKHDVWVVFKKQNTLASL

SEQ ID NO: 10 - DNA, 1759 bp, Human cyclovirus
ataccacgacttcgttgtagcacgccaaccggttccagcgcgtcaagaataatatatcccctccacagatggtacctcgaacgggttaagtagcgccgcccaatgcgggaggcaaccggaatcgacgcgggcactcgcgctgacgcgctcgtgctattccgcgtcgagccagtgacccgcatgcggcgtgatgtccaacagtactgtgcgtagattttgttttacttggaacaattatactgaattgaactacgcgctgtgtcaagagtttatcaaaaaatactgcaaatatggcatcgttggaaaggaactcgctccaacaaccaacacgcctcatctacaaggattctgtaatcttcaaaaacccatgcgcttcagtaccatcaaaaagcgtctcgataacggaatccatattgagaagtcaatggggagtgacacccaaaaccaaacgtactgttcaaaatcaggcgagttttttgaagcgggtgaccctcaatgccaagggaagagaaatgatctacaatccgtcgtggataccatccaggctgggaatgggtctttatccagtatcgcgaatgaacaccccacagcttatatacggtactttcgtggaatccaagagtacatcaagaccgttagacctattcccccacgataccacaaaactgaggtcagatattaccacggacctccagggtcagggaaatcgagacgggcactggaagaggcaacggctctcgcatctgatctaaacgatatctattacaaaccacgtgggacttggtgggacggatacaagcagcaatcatgcgtcatcatagacgacttttatgggtggataaaatatgacgaaatgttaaaaatatgcgacagatatccatataaagttcaaataaagggggggtttgaggaattcacaagtaaatacatttggattacaagtaatattgataccaatttattgtacaaatttaatgattacaacgatacagcttttgtaagacgaattgaaataaaattacttattgaatgaagtttgattaataaatgtacaccacatatcaatttttatattatagtgcgcctgagcattttcaggagcgtcgctaagcgcttgcatacccaaggcaccagtatacaaaacaacagttgaggcccctccagatatttgaattgtaggtttatactttgtttgaacagactgaacttcatatcctgctgctcctttagtctgaatagaatccaatgttgaaggaacgtaatgctgaatccccacttgcgttccacgaaagcatttagcccgatcaatagacaaaaaaccattaaaagttgcaacagttggcaactccttatgccatggaaacatacaataagcagggattaaacttgttgaattgttggaaacattctgctgcgggagtacggtaactttggctcgtgtgaaacgaactgcctcaaaagcatcagccaatgttgcaaattctgtaaaatcgctaggagtaatctcaaaggagaactgagtggtcttagaaatatcctgagtaattgaaacgaagcgagtaagcttaactctaaaagatccacgaccggcccgcttaaaaaacctccgacggcgtcggcgaagtcgccgaaccggtttacggcgccgcacaacacgtctgcgtcgagagaaaggcattgcggcactgatgcgctcgaccttgtaaagtgaaaggagcgcgcctgctacaaggtgcgctacgaagtcgtgggtaatact

SEQ ID NO: 11 - protein, 280 aa, Human cyclovirus
MSNSTVRRFCFTWNNYTELNYALCQEFIKKYCKYGIVGKELAPTTNTPHLQGFCNLQKPMRFSTIKKRLDNGIHIEKSMGSDTQNQTYCSKSGEFFEAGDPQCQGKRNDLQSVVDTIQAGNGSLSSIANEHPTAYIRYFRGIQEYIKTVRPIPPRYHKTEVRYYHGPPGSGKSRRALEEATALASDLNDIYYKPRGTWWDGYKQQSCVIIDDFYGWIKYDEMLKICDRYPYKVQIKGGFEEFTSKYIWITSNIDTNLLYKFNDYNDTAFVRRIEIKLLIE

SEQ ID NO: 12 - protein, 219 aa, Human cyclovirus
MPFSRRRRVVRRRKPVRRLRRRRRRFFKRAGRGSFRVKLTRFVSITQDISKTTQFSFEITPSDFTEFATLADAFEAVRFTRAKVTVLPQQNVSNNSTSLIPAYCMFPWHKELPTVATFNGFLSIDRAKCFRGTQVGIQHYVPSTLDSIQTKGAAGYEVQSVQTKYKPTIQISGGASTVVLYTGALGMQALSDAPENAQAHYNIKIDMWCTFINQTSFNK

SEQ ID NO: 13 - DNA, 1750 bp, Chimpanzee cyclovirus
ataccagccactttgttgcgcgatgtcaaaccgcacagtaagaaagttttgcttcacttggaataactacgaattcgacgcgtacgcaaagtgtgagacatttttaaataacttcgctaaatatggaatcgtgggagaagaactctgtccatcaacagggacgcctcacctacaggggtatgttaacctcattaaacccacacgcttcagtaccatcaagaagcatctccataacgcaatccatattgagaaggcaaacggatcagacgaacaaaaccaaacatactgtagaaaatcaggcatattttttgaaaagggagagcctatcaagcaaggacaaaggacggacctacagctgctggttaaagacacaatggagcccagcaatacccttaaagatattgccaccaaacaccctattgcatatatccggtacttcagaggtatccaagaactacggagaatggtattacctgtacctccaagaaactatcccacggaagttcgctactactggggacctcctggcagcggaaaaagtcgcagagctctacaagaagcaaccgaaatatctaccaacgggatatattataagccaaggggacaatggtgggacggctacgaacagcaatcctgcgtcatcatagacgacttctacggatggattaaatatgatgaaatacttaaaatttgcgacagatacccctacaaagtacaaataaaaggaggatacgaggaattcacaagtacacacatatggtttacatcaaacgttgacacagatttattatataaattcaacaattacataaatactgcttttgaacgacgaattactataaaagaacatattacactttcatagtattttgatttctaaatgtacaataaacatcagttataatattgaaagctgtctttcttccttccatatctggctgaccttgaaaacatattgctccacaatatactctaggtatattaatatctactcccaaacactccaaagtcggcttccaagtaatattacgtcccgttggattcgcgccttcattagcaactcctaccgttacgatgttcggtacaaatgacatagatgtacctacagtttgcgccctcaatttatgcttatcaatgcgcaaataacttgcaaaatctttaggcaacgcgatattataatgccaaggaacaacaacatacgcgggaacagatgaggttgaattatttgcaacattttgtaaaggttgaacacgaacaacaactttattcaatttaacggtttcaaaattaggagctaaacgtccatactcggtaaaatctcccatcttgaaggaacatgaccacgtagcattaatattattttcgacagacaacgtagtaattttagtaagctttgtaagcatattcccaggtttactcctaagaacacggcgacgcattctatagcgcctacgaaatctccgaaccggtttacgcacgcggcggcgacgcctaaaataacgtttgaatgccatgactcactgttgctaactgatgttgggggggaggctagcgcggcctaccggccgcttacataccgcgcctgccccccccatcccccacgggacccaggaacggcgagctaccgctcgccgaaaggagggggaaaggacggcggcctaccggccgccgcgtacacgctcagacacgctcaaggccaaaagtgaaaggcggttacgaagtggcggttacgaagtggctgggtaatact

SEQ ID NO: 14 - protein, 280 aa, Chimpanzee cyclovirus
MSNRTVRKFCFTWNNYEFDAYAKCETFLNNFAKYGIVGEELCPSTGTPHLQGYVNLIKPTRFSTIKKHLHNAIHIEKANGSDEQNQTYCRKSGIFFEKGEPIKQGQRTDLQLLVKDTMEPSNTLKDIATKHPIAYIRYFRGIQELRRMVLPVPPRNYPTEVRYYWGPPGSGKSRRALQEATEISTNGIYYKPRGQWWDGYEQQSCVIIDDFYGWIKYDEILKICDRYPYKVQIKGGYEEFTSTHIWFTSNVDTDLLYKFNNYINTAFERRITIKEHITLS

SEQ ID NO: 15 - protein, 220 aa, Chimpanzee cyclovirus
MAFKRYFRRRRRVRKPVRRFRRRYRMRRRVLRSKPGNMLTKLTKITTLSVENNINATWSCSFKMGDFTEYGRLAPNFETVKLNKVVVRVQPLQNVANNSTSSVPAYVVVPWHYNIALPKDFASYLRIDKHKLRAQTVGTSMSFVPNIVTVGVANEGANPTGRNITWKPTLECLGVDINIPRVYCGAICFQGQPDMEGRKTAFNIITDVYCTFRNQNTMKV

SEQ ID NO: 16 - DNA, 1794 bp, Human cyclovirus
ataccggcgacttcgttgcgtgctatttttatactctccctagtgcacgttggtcgaaccgtgtggaactaaccgcccgaccccgcttcgaggtggaccaccccctcccgcgcctaacggcgcggcggggccccgtccacctctacgcggggcgggctccgcgtgtcagatcacactgagatggcaaacagtacagtacgtagattcgtctttacgttaaataactatactgaagatcaataccaaaagtgcgttgagttcatttcaactaagtgcaagtatggcatcgttggaaaggaagtcggtgaggaaaatggaacccctcatctgcaaggattttgcaacctacacaaaccaatgcgctttggaaccatcaaaaagtctatcgataacgcaatccatattgagaaggcaaatggatccgacattgataaccaaaaatactgttcgaaagcaggcaacttctttgaaacgggcacaccatctcaacaaggacgtcgtacagacctggagtccctcgtggctggaatcgcggcaggggccaacaccacatccaaagtggctactgaattcccagccatgtacatcaaatatcataggggaatcaacgagtaccttaaactcgcctttcctatcaaaccaagaacagagaaaacttgggtgtactattactggggaccgactggttctggcaaatcgcgaagagcgcttgaagaagctcaatcaattaacgcagacagtatctactacaaaccgcgtggtctctggtgggacggttatcatcagcaagaaaacgttattattgacgacttctacggatggattaaatacgacgaactgctaaagatcacagaccgctacccatacaaggtgcaagtcaaagggggattcgaggaattcaccagcacccgcatatggattaccagcgaaaaggatacatgcgacttatacaaatatgaagggtataatccagcgtctttcgagcgccgaataacatgtaaaatcaacattacatatgaaaaataaattttattcaaccaataaatgaccgttgattcttatagcgtacatacaaatcctgtactagcgtaaaattagcattactaccacctacagctactcctatattctccacacaaaacattcccgtatatagtataggcaatgtcgcagtagtaccaatttcaaattcagggcgccaatcggtacgtatagtttggttcgcaggcgtagcgtcagcatctagacgtgttgcagggacgaaggacatccggccgaccttggtgcaacgaaatattttcgctttatcaatcgaaagcgccgttgggaagttgatcggagtagtaggcgtcggtttgtggtacgggagcaagcaataactgccaacgcgactcgttgaagtattcgacacattctgctggggaaatactcgcactgtctgacgcaataccttaactctctcaaaattaggtgccaggttaatatgttcggcaaaatcattcagagctacatgtagcgccaaattatactcagtgttgggtggtacaacagcctgaacggtacgcgtcaatttacaatacaatacaggggtcctacgcctatgaaaacgtctgcgacgaaccggtttgaacatgcgccggcggcgataaaccggtctgcgccggaccaaccgcctgcgtcggtatgccatggtcaactggcgcgaccttgaaaagtgaaaggcttttatagcggggtcgggcggttagagtgggggacgttacgaagtgtgttacgaagtcgccgggtaatact

SEQ ID NO: 17 - protein, 281 aa, Human cyclovirus
MANSTVRRFVFTLNNYTEDQYQKCVEFISTKCKYGIVGKEVGEENGTPHLQGFCNLHKPMRFGTIKKSIDNAIHIEKANGSDIDNQKYCSKAGNFFETGTPSQQGRRTDLESLVAGIAAGANTTSKVATEFPAMYIKYHRGINEYLKLAFPIKPRTEKTWVYYYWGPTGSGKSRRALEEAQSINADSIYYKPRGLWWDGYHQQENVIIDDFYGWIKYDELLKITDRYPYKVQVKGGFEEFTSTRIWITSEKDTCDLYKYEGYNPASFERRITCKINITYEK

SEQ ID NO: 18 - protein, 218 aa, Human cyclovirus
MAYRRRRLVRRRPVYRRRRMFKPVRRRRFHRRRTPVLYCKLTRTVQAVVPPNTEYNLALHVALNDFAEHINLAPNFERVKVLRQTVRVFPQQNVSNTSTSRVGSYCLLPYHKPTPTTPINFPTALSIDKAKIFRCTKVGRMSFVPATRLDADATPANQTIRTDWRPEFEIGTTATLPILYTGMFCVENIGVAVGGSNANFTLVQDLYVRYKNQRSFIG

SEQ ID NO: 19 - DNA, 1795 bp, Human cyclovirus
ataccgtcacttcgatgttggcgcgccccgacagtggggagtgcaaaggcgccgctacgcggcgccgtggcaaatactctcaactaacatgcctagcgggatagcggctaggggcgcgccaatgccggcttgggttcgcttcgctcacccaccggcatggcgttggcagacgtccgtggcatggcaaacagtactgtgcgccgtttctgcttcacgtggaacaattattcagaactagagtacgtgctgtgctgcgaattcattaaaaaatattgctgttatggcatcgttggaaaagaaatcgctcccaataccgggacacaccatctgcaggggttttgcaatctcgagaaacccatgcgcttcagtaccatcaagaagcgtctcgatagtagaatccatattgagaaggcagctggatccgactcagaaaaccaaacgtactgttctaagacaggcaacttttttgaatcgggaacgccaaaccaacagggacgcaggacggatcttcaggccgtcgtttctgccatacagaatggagaaatcaccacacccaaaatggtcgctgaactacatggcgccgtgtatatcaaataccatcgtggaatcgaaaaaatccttcatcagctgcatccagtgccgcctagggatataccgactgaactgcgggtgtattggggaccacccggttctgggaaatctcgcagggcgttggaggaagcacgtactctgggaggaagcatctactacaaaccacgaggtctctggtgggacggatatgaacagcagaattgtgtcatcatcgacgacttttatggttggataaaatacgacgagctattaaaaatatgtgacagatatccatacaaggttcaaataaaaggatcattccaagaattcacatccaaatatatctttattacatcaaacgttgatacatgcgattggtacaaattcgccggatacaatgtaaacgctatagagcgtcgtattacaataaaggagtacatggactaatgttccaatgtattttgattgtacaatgttaaataaacatctgtttttatcataaaataagacttgtcagattctttcaaaaatgaatttccagcaaaacacacccctccgcaaaataatcttggtaattgcgaagcctcgggagcattagaatccgggcatctcagtttaggtctaaatatatcctgacgataaaacttattcagcttgcaatcgcttcccacgcagtttatctccacattattcagcattccaatggaaggaacaaaagccattcttcctacttgggttccacggtagagtttggccttatcgacagagaggtaatcagtccaaccaactcctgttgacggaatatttttatgataaggaaacatgcaataaggaggaacagcactagtactggtgttggacacattctgctggggaatgatagtgtgcacgacgaggcggggcacgaaatattcaaaactatcttgaagtcgattaaattcagtaaaattagccagatctatatgcaattctttcaactcattttttgttacatttgcctgcacaagttgcgtctcggtgagctttactttgaaacccttggttgttccacgcttcctacggtacctacgccggtaccgcctacggcggtaaaccggtttccgtcggcgaactcgggagcggcgcacagctggcatggtcaacgaacctgttacaactggcacgcgaaccttgaaaagtgaaaggcgcgcgctacgacgtctgctacgaagtgacgggtaatact

SEQ ID NO: 20 - protein, 286 aa, Human cyclovirus
MALADVRGMANSTVRRFCFTWNNYSELEYVLCCEFIKKYCCYGIVGKEIAPNTGTHHLQGFCNLEKPMRFSTIKKRLDSRIHIEKAAGSDSENQTYCSKTGNFFESGTPNQQGRRTDLQAVVSAIQNGEITTPKMVAELHGAVYIKYHRGIEKILHQLHPVPPRDIPTELRVYWGPPGSGKSRRALEEARTLGGSIYYKPRGLWWDGYEQQNCVIIDDFYGWIKYDELLKICDRYPYKVQIKGSFQEFTSKYIFITSNVDTCDWYKFAGYNVNAIERRITIKEYMD

SEQ ID NO: 21 - protein, 230 aa, Human cyclovirus
MPAVRRSRVRRRKPVYRRRRYRRRYRRKRGTTKGFKVKLTETQLVQANVTKNELKELHIDLANFTEFNRLQDSFEYFVPRLVVHTIIPQQNVSNTSTSAVPPYCMFPYHKNIPSTGVGWTDYLSVDKAKLYRGTQVGRMAFVPSIGMLNNVEINCVGSDCKLNKFYRQDIFRPKLRCPDSNAPEASQLPRLFCGGVCFAGNSFLKESDKSYFMIKTDVYLTLYNQNTLEH

SEQ ID NO: 22 - DNA, 1699 bp, Human cyclovirus
accaggcacctcggcaccggcaccaatatggagtcaaagaggaagaagagacaaaacccagcaaagagatggtgttttacattgaacaattacacacagttagaggcaattacaattgaacaacttttatgcacagaagaagtacaatacgctattgttggagaggaaattggagaaaatggaacaccgcacttacaaggattcttcaatttaaaaaagaagaagcgacttacctccttgaaggcatggctgaatgatagagcccactacgaagaagcaaagggttccgacgaacagaacaggagatactgctctaaaagtggtaatatcctaatctctttcggctcgcctcaaaagcagggtcagagaaatgatcttgtcttggcggctgaacttttgaaagagagtggcgggaatatggcagcggttgcagatttatacccttcagccgttatcagatatgggcgtggcttacaacaatactggcagctcattggtttctctgcccgcgatttcaaaacggaggtttttgtttacgtcggcccgcccggatgtggaaagtcccgcgccgcagcagagctaggcgccgccagcggtggcaaagtgtactacaagccaagaggggagtggtgggatggctacaatggagaggccactgtaatcattgatgatttttacggatggctgaagtatgatgaattgttgagactttgtgaccgctatccacaccgggttcctgttaaagggggttttgttcagttttgtagcaagaggattatacttacttccaatattcatgtttggatgtggtatagatttgaaagctacgatgccagcgcccttatgcggcgtattaatgtgtataaattatggaatggaagtgtatgtacttttgatgatttgtgtgatgaaaaatacaacttcttgacaccattaaagtataattattaaaactttattgattttcagttgtggttcctacccatattggacgtttgaaactcacataagcctttataaggatttggtatataatagtattctgcgcaggatttgtaaagttgtttacaaaaaacgtttttaggccaaaatgaggaatatctttatatgcagaatttagccatacaccccggtttctacgttgattaaattcagcgctcacaggcgttgtagaagtagctgatgtagctaccaaccggtggaatactggccggaatttaatagtaaattttctatcattgcgccagaatttagtattattccaattttccatacttatttttgaggttggaggagtggcatcgtcaaagtccacagaggtctcattaaaaccgcctcctccagtaccttggtcaattggcatagtaggattaatttgagggagaaaggttactactatagtgttaattctataatagtcccaggttatttgcccggttaaaaattcccccaaagtgaacgttagtccaactcctttgccagctgcgtccaccctaaatgtgtcttgtttgataagacgggtggagtaagtttgtccgtagagccgcgcgggaagaaatgtgcgggcccgccttcgacctcgacgggggtcaggtcttctttggcgacgcctacctcccatttgaaaggcagggcgcgcctgctgttgccgtctgataagaaaaaaaaagcatatataagatcgaggtgccgtagtatt

SEQ ID NO: 23 - protein, 307 aa, Human cyclovirus
MESKRKKRQNPAKRWCFTLNNYTQLEAITIEQLLCTEEVQYAIVGEEIGENGTPHLQGFFNLKKKKRLTSLKAWLNDRAHYEEAKGSDEQNRRYCSKSGNILISFGSPQKQGQRNDLVLAAELLKESGGNMAAVADLYPSAVIRYGRGLQQYWQLIGFSARDFKTEVFVYVGPPGCGKSRAAAELGAASGGKVYYKPRGEWWDGYNGEATVIIDDFYGWLKYDELLRLCDRYPHRVPVKGGFVQFCSKRIILTSNIHVWMWYRFESYDASALMRRINVYKLWNGSVCTFDDLCDEKYNFLTPLKYNY

SEQ ID NO: 24 - protein, 221 aa, Human cyclovirus
MGGRRRQRRPDPRRGRRRARTFLPARLYGQTYSTRLIKQDTFRVDAAGKGVGLTFTLGEFLTGQITWDYYRINTIVVTFLPQINPTMPIDQGTGGGGFNETSVDFDDATPPTSKISMENWNNTKFWRNDRKFTIKFRPVFHRLVATSATSTTPVSAEFNQRRNRGVWLNSAYKDIPHFGLKTFFVNNFTNPAQNTIIYQILIKAYVSFKRPIWVGTTTENQ

SEQ ID NO: 25 - DNA, 1867 bp, Human cyclovirus
ataccgccacttcgtgttatataaagccatgcgttacaagctaagcttcagacaatggctgtatcaagtcgcaaacagacgaactctacgctgcgtcgcttctgttggacactcaacaactacaccgaggaagatgtcactactcttcagaaggatctcgctgagctatgtaaatttgctatatttggtagagagacatgtcctaacactggaacaaaacatctgcagggattttggtaaatatactctactcgtgcaattcgagagaaactactgcttggtgttgaccgcccgatcccgctacgaggtggaccaccccctggcgcgcttcgcgcgccggggccccgtccacctctgcgcgggacgggctccgtgtgttgtgatagtaatgtgtattgttttttcagcaatctgcagcgacccaagcgattcagtagcatacgaaaactatttaaagagcgagcgcacattgaaaaagctaaaggaagtgatttcgataataaggcatactgttctaaatctggcgaggtatggatgcacggagaacctagttctcaaggagctagaaacgatctccaagaggttgtttctgtcattgaaggaggagagagaaacattaaagcagtggcccttcagttccccacaacgtacatcaagtattttaaaggaattgaacaatacattagaatctgtcattcaagtgcagaacgagattttgctacccaagtctcgttcttctggggacctaccggatctgggaaatctagacgagcctacgaagaagcgaaagcgacaggagagccaatctactacaagcctagaggagaatggtgggatggatactgtggacacgctaatgtcatcatcgacgacttctacggatggcttaaatacgacgaattattaaaaatttgcgaccgatatccgtatagagtaccagtaaaggggggatatgaaaattttgttacaaaacgtatatggataacaagcgaaaaaccgttggaacaaatataccgatttataggttatgattgtagtagtataagaaggagactaaacacagaattgtatattggatatgaataaaatttattaaccaataaaatatttttgattgtataaagttattttagctacaagttctatctcatattgtctcctagacggcgcacccgcaccggggagttgatctttcgaccagtgcacaactccgcaataatgttgtaaactttgcgtattcgaattaagttccacgcgcggcctccactcaattttgccatattgatttcccgcaatcccagaatagccaacagcagaaagaagagcaggaacaaaccgccggaaggccccagatgtgccattatacgatttactcctatcaatagataatacaccgttgctatccacagtagttggagtaggcttatgccatggtgccacgtaataacgaggaacaggaccaacgtcagaggccacgttaaaaagcggtctaattttacatgacacactccaaatacggaatccctcaaagtttggaacaagcggcgccaactcagtaaaatcattcaaagaaggagcaatagttacagttccaccttcgtctccttgaataactacaacctgagtacgtttagcaagcaccgtaaaatttcctcttagtcgacgtgaccttcggaagcggcgccgcctagcaacacgacggcgcggcaactttcgccttatcggagcgcgtgaccgtaccactcgacgtctgaaggccatctctgacgtcacgcgataaaccggttatatcatgttgctatggaaggagaggggagccaatcagcgggcgatacgatgttacggttacgaagtggcgggtaatact

SEQ ID NO: 26 - protein, 286 aa, Human cyclovirus
MAVSSRKQTNSTLRRFCWTLNNYTEEDVTTLQKDLAELCKFAIFGRETCPNTGTKHLQGFCNLQRPKRFSSIRKLFKERAHIEKAKGSDFDNKAYCSKSGEVWMHGEPSSQGARNDLQEVVSVIEGGERNIKAVALQFPTTYIKYFKGIEQYIRICHSSAERDFATQVSFFWGPTGSGKSRRAYEEAKATGEPIYYKPRGEWWDGYCGHANVIIDDFYGWLKYDELLKICDRYPYRVPVKGGYENFVTKRIWITSEKPLEQIYRFIGYDCSSIRRRLNTELYIGYE

SEQ ID NO: 27 - protein, 222 aa, Human cyclovirus
MAFRRRVVRSRAPIRRKLPRRRVARRRRFRRSRRLRGNFTVLAKRTQVVVIQGDEGGTVTIAPSLNDFTELAPLVPNFEGFRIWSVSCKIRPLFNVASDVGPVPRYYVAPWHKPTPTTVDSNGVLSIDRSKSYNGTSGAFRRFVPALLSAVGYSGIAGNQYGKIEWRPRVELNSNTQSLQHYCGVVHWSKDQLPGAGAPSRRQYEIELVAKITLYNQKYFIG

SEQ ID NO: 28 - DNA, 1760 bp, Chicken cyclovirus
aagccgccacttcgttgtaaccatgcctcggcaaacagtcagacgtttcgtgttcacttggaacaactatcctatcgaagcatacgacaagtgcgaaaaatatctcaccaaattttgcaaatatggcatcgttggagaagaaatcgctccggaaacagggacgcctcacctacaggggttttgtaacctacataaacccacacgctttagtaccatcaagaagcatctcgataactcaatccatattgagaaggcaaatggatccgacattgacaaccaaaaatactgtagtaagtcaggcatcttttttgaaacgggacagccctctaaacaaggacagaggtcggaccttgctgtcgttagtaacaaaatcttggaaggaagtactacaatacgagatattgccacccaacatcctgaaacgtacatcagatactttcgcggcattcgagaactacaacgattggtcaacccgatctctcctcgagactttcccacacaagtatattactactacggtccgccaggaacaggtaaatccagaactgctttggaagaagcaaaaaaaattgatccgacttccatctactataaaccacgaggtctgtggtgggacggctatacaaaccagaaatgtgttattattgacgacttctacggatggataaaatacgacgaactactaaaaatttgcgaccgttatccttacaaagtacaaataaaagggggattcgaagaattcaacagcacacacatctggattacttcaaacgtcgatacagatttattgtataaatttacaggatattgtaacgctgcatttgaacgtcgtattacaataaaaaaatattttgcttaagacagtccatttattatagattgattcttcatttccacataaacattagtaactatattaaaccgcgtactccgaccagttgctgagggatcaccctgaaatcctaaagcgccacagcgaatcaaaggctgatcttgtgcagttgtagtccattgaacggtaggcttccaattaatcactggagcggtaaactcattcgacgaagtattttgagctggggatatagtattcggcacaaaactcatagtcaacggaacagtttgtggacgaatcttggctttatccacacttaacagcgttccaaaatctttaggaataggtaccccgtaatgccaagggtaaatacaatacgaaggcacagaggacgtactattgtttgacacattctgcattggaataacttttacaactactttattaacttttgtcatctcgaaattacttccgatacgagtccactcagaaaactgacccagttgaaacgaacaatcccacgtagtattaagttcattctgaacaacaatcgtatgaattcgcgtaaatttagcatacagatttccaggtttcgaataccggcctttacgtatcctacgaaatcggcgacggtacttccgcaacggtcgccgaatgcggcgacgatactttctaactcgacgatacgccatatgtagagggggagcttccctcgctaccgctcgggacaaatataaccccccttctaacaaccggtttcaacatgctttgggtagcgctcccccagctcgcccccccgccccccccccctccggtacgctccggggggggggcgtcgcgccgctgtggcgcgtgcgcgtggaaacgtgttcaaggctgaaagtgaaaggtggttacaaagttgggttacgaagtggcgggtaatact

SEQ ID NO: 29 - protein, 277 aa, Chicken cyclovirus
MPRQTVRRFVFTWNNYPIEAYDKCEKYLTKFCKYGIVGEEIAPETGTPHLQGFCNLHKPTRFSTIKKHLDNSIHIEKANGSDIDNQKYCSKSGIFFETGQPSKQGQRSDLAVVSNKILEGSTTIRDIATQHPETYIRYFRGIRELQRLVNPISPRDFPTQVYYYYGPPGTGKSRTALEEAKKIDPTSIYYKPRGLWWDGYTNQKCVIIDDFYGWIKYDELLKICDRYPYKVQIKGGFEEFNSTHIWITSNVDTDLLYKFTGYCNAAFERRITIKKYF

SEQ ID NO: 30 - protein, 222 aa, Chicken cyclovirus
MAYRRVRKYRRRIRRPLRKYRRRFRRIRKGRYSKPGNLYAKFTRIHTIVVQNELNTTWDCSFQLGQFSEWTRIGSNFEMTKVNKVVVKVIPMQNVSNNSTSSVPSYCIYPWHYGVPIPKDFGTLLSVDKAKIRPQTVPLTMSFVPNTISPAQNTSSNEFTAPVINWKPTVQWTTTAQDQPLIRCGALGFQGDPSATGRSTRFNIVTNVYVEMKNQSIINGLS

SEQ ID NO: 31 - DNA, 1838 bp, Bovine cyclovirus
atggctccgaacaagacgctgagaaggttctgctggactcttaacaactataccgaagatgacgtcgatcaacttcagaaggacctccctgagttatgtaaatttactatatttggtagagaagtttgccctacaactggaacaaaacatctgcagggattttggtaaatataacaactagtgcaattcgagagatagaaatgcccaggagagccgcccgaccccgctacgagggggtcacccctggcctcgctacgctcggccggggccccgaccccctctacgcggggcgggctccgtgcatagtgttataacatgtgtgtctcatttcagtaatctgcagcgacccaaaagattcaataacattcgcgaaatatttaaaggacgcgcgcacattgaaggagctaaaggatcagatcaggataatcaaaggtactgttctaaatctggtgaggtttggtcacacggagaaccatgtaatcaaggagctcgaagcgatctcgaagaggttgtttctatcattaaaggaggagaaagagatgttaaagcagtggcccttcagtgcccaacagcgtacattaaatattttaagggaattgaaaactacattagaatatatcatgccactccagagcgagattttaaaacagaagtgtacttcttctggggaccaactggagcaggaaaatccagaactgcacgcgaacaagcgctcgcgactggattacgggtatactacaaacctagaggagactggtgggacggatacaacggacatgaatgcgtcatcttcgatgacttctacggatggataaaatacgacgaagtacttaagatatgcgatcgttatccttatcgcgtaccagtcaaaggtggatacgagaattttattgcgaaaaaaatatggtttacgtcaaacaaaccacttgaacaaatatacaaatttattgattacgaacctagcgcatggagaaggagattgactgtagaacgagaattcttttatgaataaatctttattaacccaaaaaactcttttgattataaaatgtaacctttatttccgtttcgatttcatattgcctaatgtgtgtagaaccctcttgctggtctgcggaccataaatataacccgcaataatgcggaagagtcgccgtatttgaattgagttcaatccgcggtcgccaattaattttcccatattggggcgtcgtaatcccagcatacccagtagcagtaagaacagcaggaacaaaacgccgcacggacccactgagcccattatacgacttacacttgtcgatagtaacgatcccagttgaattaatagaagtagggccaggcttatgccaaggtgctgaataatatctaggtaccggagtagtatcagcagcaacgttaaataaagggttgaccttgactttaacagaccaaatacgataggcttcaaaataattaataaaagtggcggcttcggtaaaatccgagacagccggagatatttgcagtaggtatccagttttgccctctaccggagcaacgtataaactcttcatagaaaccgtgatatttcctgtccgctgacgtcgtctgaccttgcgtaatcgtcggcgtacgtgccgaattgcgcgtcgttttcgccttatcggtgcgcgcatgcgcactcctcggcgtctgaaggccatctctgtcgctatgcacggagcatgtctatatatagatcgggcgcggagtgggggaccaatcagcgtgcgttacggagtgttacgaagtggcgggtaatactagcccgccacttcgttgcctatataatcgcataatattcgtgctaagcccaa

SEQ ID NO: 32 - protein, 280 aa, Bovine cyclovirus
MAPNKTLRRFCWTLNNYTEDDVDQLQKDLPELCKFTIFGREVCPTTGTKHLQGFCNLQRPKRFNNIREIFKGRAHIEGAKGSDQDNQRYCSKSGEVWSHGEPCNQGARSDLEEVVSIIKGGERDVKAVALQCPTAYIKYFKGIENYIRIYHATPERDFKTEVYFFWGPTGAGKSRTAREQALATGLRVYYKPRGDWWDGYNGHECVIFDDFYGWIKYDEVLKICDRYPYRVPVKGGYENFIAKKIWFTSNKPLEQIYKFIDYEPSAWRRRLTVEREFFYE

SEQ ID NO: 33 - protein, 212 aa, Bovine cyclovirus
MRAPIRRKRRAIRHVRRRLRKVRRRQRTGNITVSMKSLYVAPVEGKTGYLLQISPAVSDFTEAATFINYFEAYRIWSVKVKVNPLFNVAADTTPVPRYYSAPWHKPGPTSINSTGIVTIDKCKSYNGLSGSVRRFVPAVLTATGYAGITTPQYGKINWRPRIELNSNTATLPHYCGLYLWSADQQEGSTHIRQYEIETEIKVTFYNQKSFLG

SEQ ID NO: 34 - DNA, 1751 bp, Capra hircus cyclovirus
atggcaaaccgtacagtaaggcgtttctgttttacgtggaacaactacgacgactctgcgtacgaaaagtgcgaaaagtttatcgaaaaattttgcaaatatggaatcgtgggagaagaatcagctccaaccactgggacgcttcacttgcaagggttctgtaatctacataagccaacacgctacagtaccatcaagaagcatctcgataacagcatccatattgagaaggcaaatgggacagacgaacaaaaccaaaaatactgttcgaaatcaggcatattttttgaatcgggtattccaaacaaacaagggacacggaatgatcttaaatctgtggtggaatcaatcaacaaagaaggtgctacgctgggaaatattgccaccgaacaccctgaaatatacatacgctatttccgtggcatccaacaactgctgcaactggtacgcccaatacgtcaacgcgatttcaagactgaggttttttattactgggggccgccaggatctgggaaatctagaagagcgcttgaggaagcgaggcaacttgacggagaatccatatactacaaaccccgaggattatggtgggatggctatgaacaacagcgcagcgttataatagacgacttctatggatggataaagtacgacgaaatgctgaaaataatggaccgctacccatacaaggtacaaataaaaggaggatttcaagaattcacatcagaaaaaatttttattacttcaaatgtcgacgtttgtgacttatacaaattcgtaggttacgaaactgccgcctttgacagacgcattacattaaaagaatacatggattaaattttcatagcctctacactctgggtaggaggatacggagacacatgtaaagatgtttgatttgaaaaaatacaataaacgtcctctacaacattaaatgtcgatgttcttccttccaaattaccctctccctgccaaacaaccaaaccagaatatatcctaggctcaaatttcgtttgatcatacggcacagtcggtttccatattatagtattcattcctcgcacagtgacgtcatcaggactgctctgagtccacgtattacatgtaaatatcatttgaccacgttccgtctgcctatatatcttagcacgatcactactaataacttgattaaaagacttcttagcctcaccacctttgtgccacggcaatatacaatatgacggaataattgtagtattagcattactaacattttgcataggaattacacgaactcgttgtttgataaatttgcagtattcaaaattcggggcaagcgatttatattcaggaaaatcagaaggaagaaagctacacgagtaagtagaagttgcattattcggaacttcatatgtagacactttagtaagtttacacataaaggagcccttagaaagacgcctacgtttccgataagttcttcgacgacgaaatctccgacgacgccccacgcgcctacgcctatatacggccctgaatcgcgccatgcctttcacttttacgctcggttaaaaataactcctcttcaggcggcagactgttgccaagtgaacgttgcgccccccccctcggccggggagcctgcccggccggcccccggcggggggagggcgtgcgtgttgaaacctgtttgggcggatgtagcggcggttacgaagtgacggttacgaagtgacggggtaatactatgcccgtcacttcgttgcaga

SEQ ID NO: 35 - protein, 278 aa, Capra hircus cyclovirus
MANRTVRRFCFTWNNYDDSAYEKCEKFIEKFCKYGIVGEESAPTTGTLHLQGFCNLHKPTRYSTIKKHLDNSIHIEKANGTDEQNQKYCSKSGIFFESGIPNKQGTRNDLKSVVESINKEGATLGNIATEHPEIYIRYFRGIQQLLQLVRPIRQRDFKTEVFYYWGPPGSGKSRRALEEARQLDGESIYYKPRGLWWDGYEQQRSVIIDDFYGWIKYDEMLKIMDRYPYKVQIKGGFQEFTSEKIFITSNVDVCDLYKFVGYETAAFDRRITLKEYMD

SEQ ID NO: 36 - protein, 231 aa, Capra hircus cyclovirus
MARFRAVYRRRRVGRRRRFRRRRTYRKRRRLSKGSFMCKLTKVSTYEVPNNATSTYSCSFLPSDFPEYKSLAPNFEYCKFIKQRVRVIPMQNVSNANTTIIPSYCILPWHKGGEAKKSFNQVISSDRAKIYRQTERGQMIFTCNTWTQSSPDDVTVRGMNTIIWKPTVPYDQTKFEPRIYSGLVVWQGEGNLEGRTSTFNVVEDVYCIFSNQTSLHVSPYPPTQSVEAMKI

SEQ ID NO: 37 - DNA, 1703 bp, Tadarida brasiliensis cyclovirus
atggcgaacgcaaccctgcgcagatttgtttttacgtggaataactataacgcggacgacatagaaaaagttgttaagttcatcaaagaaaagtgcaaatatggaatagttggtgaggaaattgcacctacaacaaatacaccacacctgcaggggttctgcaatctaaaaacacctatgcgattcaaaagcatcaaaactcatctacacacaaacatccacatagaaaaagcaaaaggtactgatctacaaaatcaagaatactgcaaaaaaggaaacaaatggatagaagaaggaacaccatgcacccaaggaaaacgcactgacttggaaaccttagtaagtgccataaacagcggaacaaatacccaacaaaaaatagctaaggaattccctatatgctacatcaaataccacagaggaatcaacgaatatctcaaactaatacatcctattgaaccccgcaaagaaaaaacatgggtctactactactggggaccaccaggcacgggaaaatcacgacgagctctggaagaagcatcccaaatcaacagcaatagtatatactacaaaccacgtggactctggtgggacggttatcatcaacaagaaaatgtcatcatagacgatttctacggatggataaagtacgacgaactcctcaaaataactgaccgctacccatataaagtacaaatcaaaggaggatttgaggagttcacatcaaaacgcatttggtttacaagcaacgtagacaccgataaattatatcattttattgattataataatgcagcgttcgaaagacgcattacatgtaaaattcacattacttaaatattcattattgattgccctgtaaaccgaacaaatacatcagtctttatattaaatgtagttgttcttccagccatatctgattgtccctgaaacgctactactccgcagcgtacacgtggcaatgaattcccattttgattaaccgttcgcacataaggtcgccattgaattgcatcaacatcctccgcaactttcccagaatgatccaaaacagacgttaatagtccaatatttggtacataggtctgactcattacatgcgtttgacgagacatctttgacttatccattgacaaatagttttgaaaatttttaggcaaatccatagcataatgccacggtgtcatggcaaaagcagggacagacgacgtagaattattagcaacattttgtaagggaacaactgtaatacgaactttaaggaagcgaacaaattcaaaatttttcgacaaagacgtaaactccttaaaatcatcaggcacaaaagagccatcccacacagaatttatattattctcaacagtaatcgtagccaccttagtaagtttacaaaacattgaacccctacgcgaacgaaccttcctccttcggttaggaaagtaccgccgcatccgacgaaccgcccgcaacggtcgtcggaaacgacgaaaaaaacgtcttaatcttaccattgctcaaggccaactggatgcgcggacgttgcaggggcgtaggcctccgctaccgctccggccacaaggtgtgtgtgggggggcattcctgcacaaacctgttttaccccctaaccagtttactcagcctccctgcaacgaagtcggatacgaagtggcgggtaatactataccgccacttcgtttcatacc

SEQ ID NO: 38 - protein, 278 aa, Tadarida brasiliensis cyclovirus
MANATLRRFVFTWNNYNADDIEKVVKFIKEKCKYGIVGEEIAPTTNTPHLQGFCNLKTPMRFKSIKTHLHTNIHIEKAKGTDLQNQEYCKKGNKWIEEGTPCTQGKRTDLETLVSAINSGTNTQQKIAKEFPICYIKYHRGINEYLKLIHPIEPRKEKTWVYYYWGPPGTGKSRRALEEASQINSNSIYYKPRGLWWDGYHQQENVIIDDFYGWIKYDELLKITDRYPYKVQIKGGFEEFTSKRIWFTSNVDTDKLYHFIDYNNAAFERRITCKIHIT

SEQ ID NO: 39 - protein, 225 aa, Tadarida brasiliensis cyclovirus
MVRLRRFFRRFRRPLRAVRRMRRYFPNRRRKVRSRRGSMFCKLTKVATITVENNINSVWDGSFVPDDFKEFTSLSKNFEFVRFLKVRITVVPLQNVANNSTSSVPAFAMTPWHYAMDLPKNFQNYLSMDKSKMSRQTHVMSQTYVPNIGLLTSVLDHSGKVAEDVDAIQWRPYVRTVNQNGNSLPRVRCGVVAFQGQSDMAGRTTTFNIKTDVFVRFTGQSIMNI

SEQ ID NO: 40 - DNA, 1200 bp, Sus scrofa cyclovirus
gtccggagggaaagcccgaaacacaggtggtgttttacgataaacaactggaccccgaccgagtgggaatctattgtggagtgtggaggcagtatagcgagataccttattatcggcaaagaggttggaaaaagcggtaccccacacttgcaagggtacgtgaatttcaagaacaaaaggcgactcagctcggtgaagcgcttacccggatttggtcgggcccatctggagccggcgagggggagccacaaagaggccagcgagtattgcaagaaagagggggattacctcgagattggcgaagattcctcttcgggtaccagatcggatcttcaagcagcagctcggattctgacggagacgtcgggaaatctgactgaagttgcggagaagatgcctgcagtatttatacgctatgggcggggtttgcgtgatttttgcggggtgatggggttgggtaaaccgcgtgattttaaaactgaagtttatgtttttattggtcctccaggatgcgggaaaacgcgggaagcttgtgcgggtgcggctgcgcgggaattgcagttatatttcaagccacgggggccttggtgggatggttataatggggaaggtgctgttattttggatgatttttatgggtgggttccatttgatgaattgctgagaattggggacaggtaccctctgagggttcctgttaagactttcatctttagtatcttataatattcaaaggtaattgcagtttcccattcgtttaggcgggtaatgaagtggttggcgtgccagggcttgttattctgaggggttccaacggatatgacgttcatggtggagtatttctttgtgtagtatgtgccagctgtgggcctcctaatgaatagtcttcttctggcatagcgccttctgtggcgtcgtcgtctccttgggcggggtcttcttctgaatatagctctgtgtctcattttggtgccgggctagtattacccggcacctcggaacccggatccacggaggtctgtagggagaaaaagtggtatcccattatggatgctccgcaccgtgtgagtggatataccgggcagtggatgatgaagcggcctcgtgttttgatgccgcaggacggggactggataactgagtttttgtggtgctacgagtgtcctgaagataaggacttttattgtcatcctattctag

SEQ ID NO: 41 - protein, 241 aa, Sus scrofa cyclovirus
VRRESPKHRWCFTINNWTPTEWESIVECGGSIARYLIIGKEVGKSGTPHLQGYVNFKNKRRLSSVKRLPGFGRAHLEPARGSHKEASEYCKKEGDYLEIGEDSSSGTRSDLQAAARILTETSGNLTEVAEKMPAVFIRYGRGLRDFCGVMGLGKPRDFKTEVYVFIGPPGCGKTREACAGAAARELQLYFKPRGPWWDGYNGEGAVILDDFYGWVPFDELLRIGDRYPLRVPVKTFIFSIL

SEQ ID NO: 42 - DNA, 22 bp, Artificial Sequence
  Primer
attgcttcgactggatggtcgt

SEQ ID NO: 43 - DNA, 22 bp, Artificial Sequence
  Primer
aacgactggatggtcgttccac

SEQ ID NO: 44 - DNA, 24 bp, Artificial Sequence
  Primer
attttccttatccgcatcaactcc

SEQ ID NO: 45 - DNA, 23 bp, Artificial Sequence
  Primer
tacaaactcaggtcgccattttg

SEQ ID NO: 46 - DNA, 20 bp, Artificial Sequence
  Primer
ggnaynccncayytncargg

SEQ ID NO: 47 - DNA, 20 bp, Artificial Sequence
  Primer
awccanccrtaraartcrtc

SEQ ID NO: 48 - DNA, 23 bp, Artificial Sequence
  Primer
ggnaynccncayytncarggntt

SEQ ID NO: 49 - DNA, 23 bp, Artificial Sequence
  Primer
tgytgytcrtanccrtcccacca

SEQ ID NO: 50 - DNA, 22 bp, Artificial Sequence
  Primer
ctacgagatattgccacccaac

SEQ ID NO: 51 - DNA, 22 bp, Artificial Sequence
  Primer
ctacatcagatactttcgcggc

SEQ ID NO: 52 - DNA, 22 bp, Artificial Sequence
  Primer
gtttagagggctgtcccgtttc

SEQ ID NO: 53 - DNA, 21 bp, Artificial Sequence
  Primer
gatggtactaaagcgtgtggg

SEQ ID NO: 54 - DNA, 23 bp, Artificial Sequence
  Primer
caggaatgcccagagtaagtaga

SEQ ID NO: 55 - DNA, 23 bp, Artificial Sequence
  Primer
ccattatcttcatcactaccgcg

SEQ ID NO: 56 - DNA, 22 bp, Artificial Sequence
  Primer
caatctacgtcaagtatgggcg

SEQ ID NO: 57 - DNA, 22 bp, Artificial Sequence
  Primer
gttcaaaacggaagtcatcgtc

SEQ ID NO: 58 - DNA, 9 bp, Artificial Sequence
  Synthetic construct
taatactaa

SEQ ID NO: 59 - DNA, 9 bp, Artificial Sequence
  Synthetic construct
taatactat

SEQ ID NO: 60 - DNA, 9 bp, Artificial Sequence
  Synthetic construct
tagtattac

SEQ ID NO: 61 - protein, 5 aa, Artificial Sequence
  Synthetic construct
WWDGY

SEQ ID NO: 62 - protein, 6 aa, Artificial Sequence
  Synthetic construct
DDFYGW

SEQ ID NO: 63 - protein, 4 aa, Artificial Sequence
  Synthetic construct
DRYP

SEQ ID NO: 64 - protein, 5 aa, Artificial Sequence
  Synthetic construct
FTLNN

SEQ ID NO: 65 - protein, 6 aa, Artificial Sequence
  Synthetic construct
TPHLQG

SEQ ID NO: 66 - protein, 3 aa, Artificial Sequence
  Synthetic construct
CSK

SEQ ID NO: 67 - DNA, 9 bp, Artificial Sequence
  Synthetic construct
taatactag

SEQ ID NO: 68 - DNA, 9 bp, Artificial Sequence
  Synthetic construct
aagtattac

SEQ ID NO: 69 - DNA, 9 bp, Artificial Sequence
  Synthetic construct
cagtattac

SEQ ID NO: 70 - DNA, 9 bp, Artificial Sequence
  Synthetic construct
tattattac

SEQ ID NO: 71 - DNA, 9 bp, Artificial Sequence
  Synthetic construct
gagtattac

SEQ ID NO: 72 - DNA, 46 bp, Artificial Sequence
  Synthetic construct
gttacgaagtggcggggtaatactatacccgccacttcgttgcacg

(19) United States
(12) Patent Application Publication
(10) Pub. No.: US 2012/0100168 A1
(43) Pub. Date: Apr. 26, 2012

(54) CYCLOVIRUS AND METHODS OF USE

(75) Inventors: Eric Delwart, San Francisco, CA (US); Li Linlin, San Francisco, CA (US)

(73) Assignee: Blood Systems, Inc., Scottsdale, AZ (US)

(21) Appl. No.: 13/012,777

(22) Filed: Jan. 24, 2011

Related U.S. Application Data
(60) Provisional application No. 61/298,151, filed on Jan. 25, 2010.

(51) Int. Cl. A61K 39/12; C07K 14/01; C07K 16/08; C12N 7/00; C12N 15/63; A61P 31/20; G01N 33/50; C12Q 1/68; C12Q 1/70; C07H 21/02; A61P 31/12; C07H 21/04; C12N 1/21

(52) U.S. Cl. 424/186.1; 536/23.72; 530/350; 530/387.9; 435/235.1; 435/320.1; 435/252.3; 436/94; 435/6.11; 435/5; 536/23.1; 536/24.33

(57) ABSTRACT

Provided herein are sequences of the genomes and encoded proteins of a novel virus, termed cyclovirus, and variants thereof. Also provided are methods of detecting cyclovirus and diagnosing cyclovirus infection, methods of treating or preventing cyclovirus infection, and methods for identifying anti-cyclovirus compounds. Further provided are vaccines and methods of preventing cyclovirus-related diseases in animals, such as pigs.

### CROSS REFERENCE TO RELATED APPLICATION

This application claims the benefit of priority under 35 U.S.C. §119(e) of U.S. Ser. No. 61/298,151 filed Jan. 25, 2010, the entire content of which is incorporated herein by reference.

### GRANT INFORMATION

This invention was made with government support under NIH Grant No. ROI HL083254. The government has certain rights in this invention.

### BACKGROUND OF THE INVENTION

1. Field of the Invention

The present invention relates generally to the discovery of cycloviruses and more specifically to methods of using the virus including methods of detecting the virus and diagnosing viral infection, methods of treating or preventing virus infection, and methods for identifying anti-viral compounds.

2. Background Information

Circoviruses are known to infect birds and pigs and can cause a wide range of severe symptoms with significant economic impact. Animal viruses with small, circular, single-stranded DNA (ssDNA) genomes comprise the Circoviridae family and the Anellovirus genus, while viruses in the Geminiviridae and Nanoviridae families infect plants. The genomes of these small viruses without a lipid envelope replicate through a rolling-circle mechanism, possibly sharing a common origin with bacterial plasmids, and show high recombination and nucleotide substitution rates.

The Circoviridae family consists of the Circovirus genus whose member species are currently known to infect only birds and pigs, and the Gyrovirus genus, including a single species, Chicken anemia virus (CAV). Circoviruses infect several avian groups, including parrots, pigeons, gulls, anserids (ducks, geese, and swans), and numerous passerines (ravens, canaries, finches, and starlings). Avian circoviruses have been associated with a variety of symptoms, including developmental abnormalities, lymphoid depletion, and immunosuppression. Mammalian circoviruses include only two closely related species, Porcine circovirus 1 and 2 (PCV1 and PCV2, respectively), infecting pigs. PCV2 has been associated with porcine circovirus-associated diseases, which can manifest as a systemic disease, respiratory disease complex, enteric disease, porcine dermatitis and nephropathy syndrome or as reproductive problems, causing great losses in the pork industry. Circovirus infections are thought to occur mainly through fecal-oral transmission.

The presence of circovirus/cycloviruses in human stool samples and in farm animal tissue also suggests the potential for frequent cross-species exposure and zoonotic transmissions. Thus, there remains a need for new circovirus/cyclovirus sequences for detecting the virus and diagnosing viral infection, as well as for treating or preventing virus infection and developing anti-viral compounds.

### SUMMARY OF THE INVENTION

The present invention is based in part on the discovery of new cycloviruses. Provided herein are sequences of the genomes and encoded proteins of a new virus, termed cyclovirus, and variants thereof. Also provided are methods of detecting cyclovirus and diagnosing cyclovirus infection, methods of treating or preventing cyclovirus infection, and methods for identifying anti-cyclovirus compounds. Further provided are vaccines and methods of preventing cyclovirus-related diseases in animals, including pigs.

Accordingly, in one embodiment, the present invention provides an isolated nucleic acid molecule. In one aspect, the isolated nucleic acid includes a nucleotide sequence having at least 60% identity to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof. In another aspect, the isolated nucleic acid includes a nucleotide sequence having at least 60% identity to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof, wherein the nucleotide sequence is at least 12, 20, 25, 30, 40, 50, 75, 100, or 200 nucleotides in length. In another aspect, the isolated nucleic acid includes a nucleotide sequence selected from the group consisting of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, and a complement thereof.

In another aspect, the isolated nucleic acid includes a nucleotide sequence that hybridizes under highly stringent conditions to at least 12, 25, 50, 100, or 150 contiguous nucleotides of a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof, wherein the hybridization reaction is incubated at 42° C. in a solution including 50% formamide, 5×SSC, and 1% SDS and washed at 65° C. in a solution including 0.2×SSC and 0.1% SDS. In an additional aspect, the nucleotide sequence hybridizes under highly stringent conditions over the full length of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof, wherein the hybridization reaction is incubated at 42° C. in a solution including 50% formamide, 5×SSC, and 1% SDS and washed at 65° C. in a solution including 0.2×SSC and 0.1% SDS.

In one aspect, the isolated nucleic acid includes a nucleotide sequence that hybridizes under highly stringent conditions to at least 12, 25, 50, 100, or 150 contiguous nucleotides of a nucleotide sequence encoding a protein selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, SEQ ID NO:30, SEQ ID NO:32, SEQ ID NO:33, SEQ ID NO:35, SEQ ID NO:36, SEQ ID NO:38, SEQ ID NO:39, SEQ ID NO:41, or a complement thereof, wherein the hybridization reaction is incubated at 42° C. in a solution including 50% formamide, 5×SSC, and 1% SDS and washed at 65° C. in a solution including 0.2×SSC and 0.1% SDS. In an additional aspect, the nucleotide sequence hybridizes under highly stringent conditions over the full length of a nucleotide sequence encoding a protein selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, SEQ ID NO:30, SEQ ID NO:32, SEQ ID NO:33, SEQ ID NO:35, SEQ ID NO:36, SEQ ID NO:38, SEQ ID NO:39, SEQ ID NO:41, or a complement thereof, wherein the hybridization reaction is incubated at 42° C. in a solution including 50% formamide, 5×SSC, and 1% SDS and washed at 65° C. in a solution including 0.2×SSC and 0.1% SDS.

In various aspects, the nucleotide sequence is at least 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof. In various aspect, the nucleotide sequence is at least 80% identical to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof.

In various aspect, the nucleotide sequence is at least 90% identical to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof. In various aspects, the nucleotide sequence is at least 95% identical to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof.

In one aspect, the nucleotide sequence includes an open reading frame. In another aspect, the nucleotide sequence includes an open reading frame encoding a protein selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, SEQ ID NO:30, SEQ ID NO:32, SEQ ID NO:33, SEQ ID NO:35, SEQ ID NO:36, SEQ ID NO:38, SEQ ID NO:39, SEQ ID NO:41, and conservative variants thereof.

In another embodiment, the present invention provides a substantially purified protein encoded by a nucleotide sequence provided herein. In one aspect, the substantially purified protein includes an amino acid sequence at least 60% identical to a sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, SEQ ID NO:30, SEQ ID NO:32, SEQ ID NO:33, SEQ ID NO:35, SEQ ID NO:36, SEQ ID NO:38, SEQ ID NO:39, SEQ ID NO:41. In another aspect, the substantially purified protein includes an amino acid sequence at least 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41. In another aspect, the substantially purified protein includes a sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41.

In another embodiment, the present invention provides a composition including a substantially purified protein provided herein. In another embodiment, the present invention provides a composition including a nucleic acid provided herein. In another embodiment, the present invention provides an isolated antibody that specifically binds to a protein provided herein. In another embodiment, the present invention provides a purified serum including a polyclonal antibody that specifically binds to a protein provided herein.

In another embodiment, the present invention provides an isolated cyclovirus including a nucleic acid molecule provided herein. In another embodiment, the present invention provides an expression vector including a nucleic acid provided herein. In another embodiment, the present invention provides a host cell including an expression vector provided herein.

In another embodiment, the present invention provides a method of detecting an cyclovirus nucleic acid. The method includes (a) contacting a sample suspected of containing an cyclovirus nucleic acid with a nucleotide sequence that hybridizes under highly stringent conditions to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof; and (b) detecting the presence or absence of hybridization.

In another embodiment, the present invention provides a method of detecting an cyclovirus nucleic acid. The method includes (a) contacting a sample suspected of containing an cyclovirus nucleic acid with a nucleotide sequence that hybridizes under highly stringent conditions to a nucleotide sequence encoding an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41, or a complement thereof; and (b) detecting the presence or absence of hybridization.

In another embodiment, the present invention provides a method of detecting a cyclovirus nucleic acid. The method includes (a) amplifying the nucleic acid of a sample suspected of containing cyclovirus nucleic acid with at least one primer that hybridizes to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof to produce an amplification product; and (b) detecting the presence of an amplification product, thereby detecting the presence of the cyclovirus nucleic acid.

In another embodiment, the present invention provides a method of detecting a cyclovirus nucleic acid. The method includes (a) amplifying the nucleic acid of a sample suspected of containing cyclovirus nucleic acid with at least one primer that hybridizes to a nucleotide sequence encoding an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41, or a complement thereof, to produce an amplification product; and (b) detecting the presence of an amplification product, thereby detecting the presence of the cyclovirus nucleic acid.

In another embodiment, the present invention provides a method of detecting a cyclovirus infection in a sample. The method includes (a) contacting a sample suspected of containing a cyclovirus protein with an antibody that specifically binds a polypeptide encoded by SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof to form a protein/antibody complex; and (b) detecting the presence of the protein/antibody complex, thereby detecting the presence of the cyclovirus protein.

In another embodiment, the present invention provides a method of detecting a cyclovirus infection in a sample. The method includes (a) contacting a sample suspected of containing a cyclovirus protein with an antibody that specifically binds to an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41, to form a protein/antibody complex; and (b) detecting the presence of the protein/antibody complex, thereby detecting the presence of the cyclovirus protein.

In another embodiment, the present invention provides a kit for detecting a cyclovirus nucleic acid. The kit includes at least one nucleic acid molecule that hybridizes under highly stringent conditions to a nucleic acid molecule provided herein. In another embodiment, the present invention provides a kit for detecting a cyclovirus nucleic acid. The kit includes at least one oligonucleotide primer that hybridizes to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof, under highly stringent PCR conditions. In another embodiment, the present invention provides a kit for detecting a cyclovirus in a sample. The kit includes an antibody specifically binds to a protein provided herein.

In another embodiment, the present invention provides a method of assaying for an anti-cyclovirus compound. The method includes (a) contacting a sample containing a cyclovirus with a test compound, the cyclovirus including a genome that hybridizes under highly stringent conditions to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof; and (b) determining whether the test compound inhibits cyclovirus replication, wherein inhibition of cyclovirus replication indicates that the test compound is an anti-cyclovirus compound.

In another embodiment, the present invention provides a method of treating or preventing a cyclovirus infection in a subject. The method includes administering to the subject an antigen encoded by a cyclovirus, the cyclovirus including a genome that hybridizes under highly stringent conditions to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof; thereby treating or prevention infection in the subject. In another embodiment, the present invention provides a method of treating or preventing a cyclovirus infection in a subject. The method includes administering to the subject an antigen encoded by a cyclovirus, wherein the antigen includes an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41, thereby treating or prevention infection in the subject.

In another embodiment, the present invention provides a vaccine for the prevention of gastrointestinal tract, respiratory, nervous system or blood infection in a subject. The vaccine includes a cyclovirus or at least one cyclovirus antigen from the cyclovirus which induces a gastrointestinal tract, respiratory, nervous system or blood infection in a subject and a pharmacologically acceptable carrier wherein the cyclovirus has gastrointestinal tract, respiratory, nervous system or blood infection inducing characteristics. In one aspect, the cyclovirus antigen has an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41.

In another embodiment, the present invention provides a method for detecting and serotyping cyclovirus in a sample. The method includes (a) contacting a first portion of the sample with a first pair of primers in a first amplification protocol, wherein the first pair of primers have an associated first characteristic amplification product if a cyclovirus is present in the sample; (b) determining whether or not the first characteristic amplification product is present; (c) contacting a second portion of the sample with a second pair of primers in a second amplification protocol, wherein the second pair of primers have an associated second characteristic amplification product if a cyclovirus is present in the sample and wherein the second pair of primers are different from the first pair of primers; (d) determining whether or not the second characteristic amplification product is present; (e) based on whether or not the first and second characteristic amplification product are present, selecting one or more subsequent pair of primers and contacting the one or more subsequent pair of primers with additional portions of the sample in subsequent amplification protocols, wherein each subsequent pair of primers is different from each pair of primers already used and wherein each subsequent pair of primers has an associated subsequent characteristic amplification product if a cyclovirus is present in the sample; (f) determining whether or not the associated characteristic amplification product for each subsequent pair of primers used is present; (g) repeating steps e) and f) for one or more subsequent pairs of primers if the cyclovirus cannot be serotyped based on the determinations of steps b), d), and f) until the cyclovirus can be serotyped, wherein the one or more subsequent pairs of primers are different from all pairs of primers used in earlier amplification protocols; and (h) determining the serotype or groups of serotypes of the cyclovirus that may be present in the sample.

In one aspect, the cyclovirus has a genome including a nucleic acid sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof. In another aspect, the cyclovirus has a genome including a nucleic acid sequence encoding an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41. In another aspect, the first, second, and any subsequent amplification protocols are polymerase chain reactions or reverse-transcription polymerase chain reactions. In another aspect, the detecting and serotyping of the cyclovirus in the sample is used to diagnose a viral disease or medical condition. In an additional aspect, the viral disease or medical condition is an gastrointestinal tract infection.

In another embodiment, the present invention provides a method for detecting the presence of a cyclovirus in a sample. The method includes (a) purifying RNA contained in the sample; (b) reverse transcribing the RNA with primers effective to reverse transcribe cyclovirus RNA to provide a cDNA; (c) contacting at least a portion of the cDNA with (i) a composition that promotes amplification of a nucleic acid and (ii) an oligonucleotide mixture wherein the mixture includes at least one oligonucleotide that hybridizes to a highly conserved sequence of the sense strand of a cyclovirus nucleic acid and at least one oligonucleotide that hybridizes to a highly conserved sequence of the antisense strand of a cyclovirus nucleic acid; (d) carrying out an amplification procedure on the amplification mixture such that, if a cyclovirus is present in the sample, a cyclovirus amplicon is produced whose sequence includes a nucleotide sequence of at least a portion of the cyclovirus genome; and (e) detecting whether an amplicon is present; wherein the presence of the amplicon indicates that a cyclovirus is present in the sample.

In one aspect, the cyclovirus has a genome including a nucleic acid sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof. In another aspect, the cyclovirus has a genome including a nucleic acid sequence encoding an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41. In one aspect, the detecting of the cyclovirus in the sample is used to diagnose a viral disease or medical condition. In an additional aspect, the viral disease or medical condition is an gastrointestinal tract infection.

In another embodiment, the present invention provides a vaccine for protecting an animal from infection by a cyclovirus. The vaccine is selected from the group consisting of (a) a genetically modified cyclovirus encoded by the isolated polynucleotide molecule provided herein; and (b) a viral vector including the isolated polynucleotide molecule provided herein; wherein the vaccine is in an amount effective to produce immunoprotection against infection by a cyclovirus and the vaccine includes a vaccine carrier acceptable for human or veterinary use.

In another embodiment, the present invention provides a vaccine for the prevention of a systemic disease, respiratory disease complex, enteric disease, postweaning multisystemic wasting syndrome, porcine dermatitis and nephropathy syndrome or reproductive disorders in porcine. The vaccine includes a cyclovirus or at least one cyclovirus antigen from the cyclovirus which induces a systemic disease, respiratory disease complex, enteric disease, porcine dermatitis and nephropathy syndrome or reproductive disorders in porcine and a pharmacologically acceptable carrier wherein the cyclovirus has systemic disease, respiratory disease complex, enteric disease, postweaning multisystemic wasting syndrome, porcine dermatitis and nephropathy syndrome or reproductive disorders inducing characteristics.

In another embodiment, the present invention provides a method of protecting an animal from infection with a strain of cyclovirus. The method including administering to the animal, an immunogenically protective amount of the vaccine provided herein, thereby stimulating an immunoprotective response against cyclovirus in the animal. In one aspect, the animal is a mammal or a bird. In another aspect, the animal is selected from the group consisting of human, bird, pig, cow, sheep, goat, camel, chicken, and chimpanzee. In another aspect, the animal is a pig. In another aspect, the bird is a chicken.

In another embodiment, the present invention provides a composition including a pharmaceutically acceptable vehicle and at least one cyclovirus immunogen selected from the group consisting of an inactivated immunogenic cyclovirus, an attenuated immunogenic cyclovirus, and an isolated immunogenic cyclovirus polypeptide.

In another embodiment, the present invention provides a method of treating or preventing a cyclovirus-associated disease or disorder in an animal including administering to the animal a therapeutically effective amount of a composition provided herein. In one aspect, the cyclovirus-associated disease or disorder is selected from the group consisting of systemic disease, respiratory disease complex, enteric disease, postweaning multisystemic wasting syndrome, porcine dermatitis and nephropathy syndrome and reproductive disorders. In another aspect, the animal is selected from the group consisting of human, bird, pig, cow, sheep, goat, camel, chicken, and chimpanzee.

### BRIEF DESCRIPTION OF THE DRAWINGS

FIG. 1 shows phylogenetic analysis of the translated Rep sequence amplified by pan-Rep PCR. Cycloviruses sequences are grouped into 25 species as shown on the right. Cycloviruses in the same species are defined as having >85% identity in Rep region and are labeled by vertical bars 1-25. The bar represents 5% estimated phylogenetic divergence. The country of origin and the sample type of the color-highlighted sequences are shown in the box.

FIG. 2 shows genomic organizations of (A) circoviruses and (B) cycloviruses. The 2 major ORFs, encoding the putative replication associated protein (Rep) and the putative capsid protein (Cap), and other ORFs with a coding capacity greater than 100 amino acids are shown. The locations of the stem-loop structures are marked.

FIG. 3 shows phylogenetic analysis of 15 Circoviridae replicase proteins from 12 human and 3 chimpanzee stools. Outlier taxas are non-circoviridae Rep proteins. Sample designation is the same as in FIG. 1.

FIG. 4 shows stem-loop of Cyclovirus prototype CyCV1-PK5006 (A), and nonamer sequences and stem length of the stem-loop structure for circoviruses and cycloviruses (B).

FIGS. 5A-5I show exemplary sequences from 9 new cyclovirus species discovered from human or chimpanzee feces. FIG. 5J shows exemplary sequences from 1 new cyclovirus species discovered from chicken muscle. FIGS. 5K-5N show additional exemplary cyclovirus sequences. FIGS. 5P-5Q show additional sequences.

FIG. 6 shows phylogenetic analysis of pan-Rep translation products together with Rep proteins from plant and animal viruses, bacteria, protozoa and environmental Circovirus-like genome (Genbank accession No. FJ959077-86), falling outside of the circovirus and cyclovirus Glade.

FIG. 7 shows genomic organization of the cycloviruses, circoviruses and circovirus-like virus discovered in animal tissues. The two major ORFs, encoding the putative replication associated protein (Rep) and the putative capsid cpotein (Cap), and other ORFs with a coding capacity greater than 100 amino acids were shown.

FIG. 8 shows phylogenetic analysis of chicken cyclovirus and circovirus, and representative cyclovirus and circovirus species based on the complete amino acid sequence of Rep protein using the neighbor joining method with 1,000 bootstrap replicates. The bar represents 10% estimated phylogenetic divergence. The GenBank accession numbers of the Rep sequences for viruses used in the phylogenetic analysis are as follows: BFDV (AF071878), CaCV (AJ301633), CoCV (AF252601), DuCV (DQ100076), GoCV (AJ304456), GuCV (DQ845074), FiCV (DQ845075), RaCV (DQ146997), StCV (DQ172906), SwCV (EU056310), PCV1 (AY660574), PCV2 (AY424401), CAV (M55918), Milk vetch dwarf virus (AB009047), Pepper golden mosaic virus (U57457), cycloviruses (GQ404844-GQ404850, GQ404854-GQ404858, HM228874, and HM228875).

FIG. 9 shows porcine circovirus 2 genotype. Phylogenetic analysis was based on the nucleotide sequence of the full-length ORF2 of representative PCV2 strains.

### DETAILED DESCRIPTION OF THE INVENTION

The present invention is based in part on the discovery of new cycloviruses. Provided herein are sequences of the genomes and encoded proteins of a new virus, termed cyclovirus, and variants thereof. Also provided are methods of detecting cyclovirus and diagnosing cyclovirus infection, methods of treating or preventing cyclovirus infection, and methods for identifying anti-cyclovirus compounds. Further provided are vaccines and methods of preventing cyclovirus-related diseases in animals, including pigs.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Using viral metagenomics, the present invention provides circovirus-like DNA sequences and characterized 15 circular viral DNA genomes in stool samples from humans in Pakistan, Nigeria, Tunisia, and the United States and from wild chimpanzees. Distinct genomic features and phylogenetic analysis indicate that some viral genomes were part of a previously unrecognized genus in the Circoviridae family the inventors tentatively named “Cyclovirus” whose genetic diversity is comparable to that of all the known species in the Circovirus genus. Circoviridae detection in the stools of U.S. adults was limited to porcine circoviruses which were also found in most U.S. pork products. To determine whether the divergent cycloviruses found in non-U.S. human stools were of dietary origin, the inventors genetically compared them to the cycloviruses in muscle tissue samples of commonly eaten farm animals in Pakistan and Nigeria. Limited genetic overlap between cycloviruses in human stool samples and local cow, goat, sheep, camel, and chicken meat samples indicated that the majority of the 25 Cyclovirus species identified might be human viruses. The present invention provides that the genetic diversify of small circular DNA viral genomes in various mammals, including humans, is significantly larger than previously recognized, and frequent exposure through meat consumption and contact with animal or human feces provides ample opportunities for cyclovirus transmission. Determining the role of cycloviruses, found in 7 to 17% of non-U.S. human stools and 3 to 55% of non-U.S. meat samples tested, in both human and animal diseases is now facilitated by knowledge of their genomes.

The present invention provides highly diverse, circovirus-like, circular DNA viral genomes discovered in human and chimpanzee stool samples, and the present invention also provides their inclusion in a new genus of the Circoviridae family that we tentatively name “Cyclovirus” pending review by the International Committee on Taxonomy of Viruses (ICTV). Cycloviruses were also found to be prevalent in the muscle tissue of farm animals, such as chickens, cows, sheep, goats, and camels. The Cyclovirus species found in human stool samples and in animal meat samples showed limited genetic overlap, suggesting that most of the cycloviruses found in human stool samples are not from consumed animal meat. Rather, these cycloviruses in human stools might cause human enteric infections.

The identifications of cycloviruses provide methods of detecting the virus, its genome, transcripts, and proteins including structural and non-structural proteins. Antibodies (polyclonal and monoclonal) made to antigens from any of these viral proteins can be used to detect the antigen or protein as well as to isolate the antigens and to remove virus, proteins, or nucleic acids from a sample, e.g., a blood sample. Antibodies to cyclovirus antigens can be used in diagnostic assays to detect viral infection. Any suitable sample, including blood, saliva, sputum, etc., can be used in a diagnostic assay of the invention. Such antibodies can also be used in therapeutic applications to inhibit or prevent viral infection.

The cyclovirus antigens of the invention can also be used in diagnostic application to detect anti-cyclovirus antigen antibodies in infected or exposed subjects. Cyclovirus antigens of the invention can also be used therapeutically, as prophylactic vaccines or vaccines for acute or latent infections, e.g., whole virus vaccines, protein or subunit vaccines, and nucleic acid vaccines encoding viral proteins, ORFs or genomes for intracellular expression and secretion or cell surface display, or can be targeted to specific cell types using promoters and vectors.

The cyclovirus virus, nucleic acids and proteins of the invention can be used to assay for antiviral compounds, including compounds that inhibit (1) viral interactions at the cell surface, e.g., viral transduction (e.g., block viral cell receptor binding or internalization); (2) viral replication and gene expression, e.g., viral replication (e.g., by inhibiting non-structural protein activity, origin activity, or primer binding), viral transcription (promoter or splicing inhibition, nonstructural protein inhibition), viral protein translation, protein processing (e.g., cleavage or phosphorylation); and (3) viral assembly and egress, e.g., viral packaging, and virus release.

“Cyclovirus” refers to both the genetic components of the virus, e.g., the genome (positive or negative) and RNA transcripts thereof (either sense or antisense), proteins encoded by the genome (including structural and nonstructural proteins), and viral particles. Cyclovirus nucleic acids may be isolated from a host including, but not limited to, primate, e.g., human; rodent, e.g., rat, mouse, hamster; cow, pig, horse, sheep, or any mammal. The nucleic acids and proteins of the invention include both naturally occurring and recombinant molecules.

Disclosed cyclovirus nucleic acids can be used to produce infectious clones, e.g., for production of recombinant viral particles, including empty capsids or capsids containing a recombinant (e.g., wild type or further comprising a heterologous nucleic acid) or modified (e.g., mutated) cyclovirus genome, which may be replication competent or incompetent, using the methods disclosed in U.S. Pat. Nos. 6,558,676; 6,132,732; 6,001,371; 5,916,563; 5,827,647; 5,508,186; 6,379,885; 6,287,815; 6,204,044; and 5,449,608. Such particles are useful as gene transfer vehicles, and as vaccines, and for use in diagnostic applications and for drug discovery assays for antiviral compounds, as discussed below.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from an eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 75% identity, preferably 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

There are several methods available and well known to those skilled in the art to obtain full-length DNAs, or extend short DNAs, for example those based on the method of Rapid Amplification of cDNA Ends (RACE). Another sequencing method is based on detecting the activity of DNA polymerase with a chemiluminescent enzyme. Essentially, the method allows sequencing of a single strand of DNA by synthesizing the complementary strand along it, one base pair at a time, and detecting which base was actually added at each step. The template DNA is immobilized, and solutions of A, C, G, and T nucleotides are added sequentially. Light is produced only when the nucleotide solution compliments the first unpaired base of the template. The sequence of solutions which produce chemiluminescent signals allows the determination of the sequence of the template.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman (1981) Adv. Appl. Math. 2:482, by the homology alignment algorithm of Needleman & Wunsch (1970) J. Mol. Biol. 48:443, by the search for similarity method of Pearson & Lipman (1988) Proc. Nat'l. Acad. Sci. USA 85:2444, by computerized implementations of these algorithms (GAP, BESTFIT, FASTA, and TFASTA in the Wisconsin Genetics Software Package (Genetics Computer Group, 575 Science Dr., Madison, Wis.), or by manual alignment and visual inspection (see, e.g., Current Protocols in Molecular Biology, Ausubel et al., eds. (New York, Wiley 1994, and 1995 supplement).

A preferred example of algorithm that is suitable for determining percent sequence identity and sequence similarity are the BLAST and BLAST 2.0 algorithms, which are described in Altschul et al. (1977) Nuc. Acids Res. 25:3389-3402 and Altschul et al. (1990) J. Mol. Biol. 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, Ausubel et al., eds. (New York, Wiley 1994).

For polymerase chain reactions or PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, see e.g., Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al. (1990) Nature 348:552-554).

For preparation of antibodies, e.g., recombinant, monoclonal, or polyclonal antibodies, many technique known in the art can be used (see, e.g., Kohler & Milstein (1975) Nature 256:495-497; Kozbor et al. (1983) Immunology Today 4: 72; Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,946,778 and 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016; Marks et al. (1992) Bio/Technology 10:779-783; Lonberg et al. (1994) Nature 368:856-859; Morrison (1994) Nature 368:812-13; Fishwild et al. (1996) Nature Biotechnology 14:845-51; Neuberger (1996) Nature Biotechnology 14:826; and Lonberg & Huszar (1995) Intern. Rev. Immunol. 13:65-93). Alternatively, phage display technology can be used to identify antibodies and heteromeric Fab fragments that specifically bind to selected antigens (see, e.g., McCafferty et al. (1990) Nature 348:552-554; Marks et al. (1992) Biotechnology 10:779-783). Antibodies can also be made bispecific, i.e., able to recognize two different antigens (see, e.g., WO 93/08829, Traunecker et al. (1991) EMBO J. 10:3655-3659; and Suresh et al. (1986) Methods in Enzymology 121:210). Antibodies can also be heteroconjugates, e.g., two covalently joined antibodies, or immunotoxins (see, e.g., U.S. Pat. No. 4,676,980; WO 91/00360; and WO 92/200373).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al. (1986) Nature 321:522-525; Riechmann et al. (1988) Nature 332:323-327; Verhoeyen et al. (1988) Science 239:1534-1536; and Presta (1992) Curr. Op. Struct. Biol. 2:593-596), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The antibody can be conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to an cyclovirus, polymorphic variants, alleles, orthologs, and conservatively modified variants, or splice variants, or portions thereof, can be selected to obtain only those polyclonal antibodies that are specifically immunoreactive with cyclovirus and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The phrase “functional effects” in the context of assays for testing compounds that modulate activity of an cyclovirus includes the determination of a parameter that is indirectly or directly under the influence of an cyclovirus, e.g., a phenotypic or chemical effect, such as the ability to increase or decrease viral genome replication, viral RNA and protein production, virus packaging, viral particle production (particularly replication competent viral particle production), cell receptor binding, viral transduction, cellular infection, antibody binding, inducing a cellular or humoral immune response, viral protein enzymatic activity, etc. “Functional effects” include in vitro, in vivo, and ex vivo activities. Such functional effects can be measured by any means known to those skilled in the art, e.g., changes in spectroscopic characteristics (e.g., fluorescence, absorbance, refractive index); hydrodynamic (e.g., shape); chromatographic; or solubility properties for a protein; measuring inducible markers or transcriptional activation of a protein; measuring binding activity or binding assays, e.g., binding to antibodies; measuring changes in ligand or substrate binding activity; measuring viral replication; measuring cell surface marker expression; measurement of changes in protein levels; measurement of RNA stability; identification of downstream or reporter gene expression (CAT, luciferase, β-gal, GFP and the like), e.g., via chemiluminescence, fluorescence, colorimetric reactions, antibody binding, and inducible markers.

“Inhibitors,” “activators,” and “modulators” of cyclovirus nucleic acid and polypeptide sequences are used to refer to activating, inhibitory, or modulating molecules identified using in vitro and in vivo assays of the cyclovirus nucleic acid and polypeptide sequences. Inhibitors are compounds that, e.g., bind to, partially or totally block activity, decrease, prevent, delay activation, inactivate, desensitize, or down regulate the activity or expression of cyclovirus, e.g., antagonists.

“Activators” are compounds that increase, open, activate, facilitate, enhance activation, sensitize, agonize, or up regulate cyclovirus activity, e.g., agonists Inhibitors, activators, or modulators also include genetically modified versions of cyclovirus, e.g., versions with altered activity, as well as naturally occurring and synthetic ligands, substrates, antagonists, agonists, antibodies, peptides, cyclic peptides, nucleic acids, antisense molecules, ribozymes, small chemical molecules and the like. Such assays for inhibitors and activators include, e.g., expressing cyclovirus in vitro, in cells, or cell membranes, applying putative modulator compounds, and then determining the functional effects on activity, as described above.

Samples or assays comprising cyclovirus that are treated with a potential activator, inhibitor, or modulator are compared to control samples without the inhibitor, activator, or modulator to examine the extent of inhibition. Control samples (untreated with inhibitors) are assigned a relative protein activity value of 100% Inhibition of cyclovirus can be achieved when the activity value relative to the control is about 80%, preferably 50%, more preferably 25-0%. Activation of cyclovirus can be achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulation tumor cell proliferation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

An “siRNA” molecule or an “RNAi” molecule refers to a nucleic acid that forms a double stranded RNA, which double stranded RNA has the ability to reduce or inhibit expression of a gene or target gene when the siRNA expressed in the same cell as the gene or target gene. The term “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length. See also WO 2003/076592, herein incorporated by reference in its entirety.

An siRNA molecule or RNAi molecule is “specific” for a target nucleic acid if it reduces expression of the nucleic acid by at least about 10% when the siRNA or RNAi is expressed in a cell that expresses the target nucleic acid.

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology, Ausubel et al., eds. (New York, Wiley 1994).

Other techniques that can be used to identify known and previously uncharacterized cyclovirus isolates, including representational difference analysis (RDA), DNA microarrays and use of degenerate PCR primers or other methods well known to those of skill in the art. Other methods for determining the sequence of an cyclovirus include, for example, sequence independent single primer amplification of nucleic acids in serum (DNase-SISPA). In this method, DNA is isolated directly from environmental samples and sequenced. This method first removes contaminating human DNA in plasma or serum by DNase digestion. Viral nucleic acids protected from DNase digestion by their viral coats are then converted into double stranded DNA (dsDNA) using random primers. The dsDNA is then digested by a 4 base pair specific restriction endonuclease resulting in two overhanging bases to which are ligated a complementary oligonucleotide linker. A PCR primer complementary to the ligated linker is then used to PCR amplify the sequences between the restriction sites. The PCR products are analyzed by PAGE and distinct DNA bands are extracted, subcloned and sequenced. Similarity to known viruses is then tested using BLASTn (for nucleic acid similarity) and tBLASTx (for protein similarity). The DNase-SISPA method does not require foreknowledge of the viral sequences being amplified and can therefore theoretically amplify more divergent members of known viral families than nucleic acid sequence similarity-dependent approaches using degenerate primers or microarrays. There are several methods available and well known to those skilled in the art to obtain full-length DNAs, or extend short DNAs, for example, those based on the method of Rapid Amplification of cDNA Ends (RACE) and large scale sequencing.

To make a cDNA library to clone cyclovirus genes expressed by the genome, the source used should be rich in the RNA of choice. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman (1983) Gene 25:263-269; Sambrook et al., supra; Ausubel et al., supra).

For a genomic library, the DNA is extracted from the tissue and optionally mechanically sheared or enzymatically digested. The fragments are then separated by gradient centrifugation from undesired sizes and are constructed in suitable vectors. These vectors are packaged in vitro. Recombinant vectors can be analyzed, e.g., by plaque hybridization as described in Benton & Davis (1977) Science 196:180-182. Colony hybridization is carried out as generally described in Grunstein et al. (1975) Proc. Natl. Acad. Sci. USA., 72:3961-3965.

A preferred method of isolating cyclovirus and orthologs, alleles, mutants, polymorphic variants, splice variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR and RT-PCR) and ligase chain reaction (LCR) can be used to amplify nucleic acid sequences directly from mRNA, from cDNA, from genomic libraries or cDNA libraries. Degenerate oligonucleotides can be designed to amplify homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of Cyclovirus encoding mRNA in physiological samples, for nucleic acid sequencing, or for other purposes. Genes amplified by the PCR reaction can be purified from agarose gels and cloned into an appropriate vector.

Gene expression of cycloviruses can also be analyzed by techniques known in the art, e.g., reverse transcription and amplification of mRNA, isolation of total RNA or poly A RNA, northern blotting, dot blotting, in situ hybridization, RNase protection, high density polynucleotide array technology, e.g., and the like.

Nucleic acids encoding an cyclovirus genome or protein can be used with high density oligonucleotide array technology to identify cyclovirus, orthologs, alleles, conservatively modified variants, and polymorphic variants in this invention. In the case where the homologs being identified are linked to modulation of the cell cycle, they can be used with oligonucleotide array as a diagnostic tool in detecting the disease in a biological sample, see, e.g., Gunthand et al. (1998) AIDS Res. Hum. Retroviruses 14: 869-876; Kozal et al. (1996) Nat. Med. 2:753-759; Matson et al. (1995) Anal. Biochem. 224:110-106; Lockhart et al. (1996) Nat. Biotechnol. 14:1675-1680; Gingeras et al. (1998) Genome Res. 8:435-448; Hacia et al. (1998) Nucleic Acids Res. 26:3865-3866.

The gene of choice is typically cloned into intermediate vectors before transformation into prokaryotic or eukaryotic cells for replication and/or expression. These intermediate vectors are typically prokaryote vectors, e.g., plasmids, or shuttle vectors.

To obtain high level expression of a cloned gene or genome, one typically subclones the nucleic acid into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al, supra. Bacterial expression systems for expressing the protein are available in, e.g., E. coli, Bacillus sp., and Salmonella (Palva et al. (1983) Gene 22:229-235; Mosbach et al. (1983) Nature 302:543-545. Kits for such expression systems are commercially available. Eukaryotic expression systems for mammalian cells, yeast, and insect cells are well known in the art and are also commercially available. In one preferred embodiment, retroviral expression systems are used in the present invention.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence encoding the nucleic acid of choice and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, β-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMT010/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

In one embodiment, the vectors of the invention have a regulatable promoter, e.g., tet-regulated systems and the RU-486 system (see, e.g., Gossen & Bujard (1992) PNAS 89:5547; Oligino et al. (1998) Gene Ther. 5:491-496; Wang et al. (1997) Gene Ther. 4:432-441; Neering et al. (1996) Blood 88:1147-1155; and Rendahl et al. (1998) Nat. Biotechnol. 16:757-761). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a sequence of choice under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical; any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein, which are then purified using standard techniques (see, e.g., Colley et al. (1989) J. Biol. Chem. 264:17619-17622; Guide to Protein Purification, in Methods in Enzymology, vol. 182 (Deutscher, ed., 1990)). Transformation of eukaryotic and prokaryotic cells are performed according to standard techniques (see, e.g., Morrison (1977) J. Bact. 132:349-351; Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing cyclovirus proteins and nucleic acids.

After the expression vector is introduced into the cells, the transfected cells are cultured under conditions favoring expression of the protein of choice, which is recovered from the culture using standard techniques identified below.

Either naturally occurring or recombinant cyclovirus proteins can be purified for use in diagnostic assays, for making antibodies (for diagnosis and therapy) and vaccines, and for assaying for anti-viral compounds. The protein may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant protein is being purified. For example, proteins having established molecular adhesion properties can be reversible fused to the protein. With the appropriate ligand or substrate, a specific protein can be selectively adsorbed to a purification column and then freed from the column in a relatively pure form. The fused protein is then removed by enzymatic activity. Finally, protein could be purified using immunoaffinity columns. Recombinant protein can be purified from any suitable source, include yeast, insect, bacterial, and mammalian cells.

Methods for production and purification of recombinant protein from a bacterial or eukaryotic (e.g., yeast, mammalian cell, and the like) system are well known in the art. Recombinant proteins are expressed by transformed host cells, (e.g., bacteria) in large amounts, typically after promoter induction; but expression can be constitutive. Promoter induction with IPTG is one example of an inducible promoter system. Host cells are grown according to standard procedures in the art. Where the host cell is a bacterial cell, fresh or frozen bacteria cells are used for isolation of protein.

Recombinant proteins, particularly when expressed in bacterial host cells, may form insoluble aggregates (“inclusion bodies”). Several protocols are suitable for purification of protein inclusion bodies. For example, purification of inclusion bodies typically involves the extraction, separation and/or purification of inclusion bodies by disruption of bacterial cells, e.g., by incubation in a buffer of 50 mM Tris/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Human proteins are separated from other bacterial proteins by standard separation techniques, e.g., with Ni-NTA agarose resin.

Alternatively, where the host cell is a bacterium, it is possible to purify recombinant protein from bacteria periplasm. After lysis of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Standard protein separation techniques for purifying proteins are also contemplated in the present invention. Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the protein can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed.

The protein can also be separated from other proteins on the basis of its size, net surface charge, hydrophobicity, and affinity for ligands or substrates. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

In addition to the detection of an cyclovirus gene and gene expression using nucleic acid hybridization technology, one can also use immunoassays to detect cyclovirus proteins, virus, and nucleic acids of the invention. Such assays are useful for, e.g., therapeutic and diagnostic applications. Immunoassays can be used to qualitatively or quantitatively analyze protein, virus, and nucleic acids. A general overview of the applicable technology can be found in Harlow & Lane, Antibodies: A Laboratory Manual (1988).

Methods of producing polyclonal and monoclonal antibodies that react specifically with cyclovirus protein, virus and nucleic acids are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein (1975) Nature 256:495-497). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al. (1989) Science 246:1275-1281; Ward et al. (1989) Nature 341:544-546).

A number of immunogens comprising portions of an cyclovirus protein, virus or nucleic acid may be used to produce antibodies specifically reactive with the cyclovirus. For example, a recombinant cyclovirus protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into a subject capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The immune response to the immunogen preparation is monitored by taking test bleeds and determining the titer of reactivity to the beta subunits. When appropriately high titers of antibody to the immunogen are obtained, blood is collected and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from a subject immunized with a desired antigen are immortalized, commonly by fusion with a myeloma cell (see, Kohler & Milstein (1976) Eur. J. Immunol. 6:511-519). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al. (1989) Science 246:1275-1281.

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-cyclovirus proteins and nucleic acids, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular cyclovirus protein can also be made by subtracting out other cross-reacting proteins, e.g., from other human cycloviruses or other non-human cycloviruses. In this manner, antibodies that bind only to the protein of choice may be obtained.

Once the specific antibodies against an cyclovirus protein, virus or nucleic acid in are available, the antigen can be detected by a variety of immunoassay methods. In addition, the antibody can be used therapeutically. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

Protein, in this case cyclovirus protein which is either associated with or separate from an cyclovirus viral particle, can be detected and/or quantified using any of a number of well recognized immunological binding assays (see, e.g., U.S. Pat. Nos. 4,366,241; 4,376,110; 4,517,288; and 4,837,168). Cyclovirus viral particles may be detected based on an epitope defined by the viral proteins as presented in a viral particle and/or an epitope defined by a viral protein that is separate from a viral particle (e.g., such as may be present in an infected cell). As used in this context, “antigen” is meant to refer to an cyclovirus polypeptide as well as cyclovirus viral particles. For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice. The antibody may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled cyclovirus protein nucleic acid or a labeled anti-cyclovirus antibody. Alternatively, the labeling agent may be a third moiety, such a secondary antibody, which specifically binds to the antibody/antigen complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al. (1973) J. Immunol. 111:1401-1406; Akerstrom et al. (1985) J. Immunol. 135:2589-2542). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Immunoassays for detecting cyclovirus protein, virus and nucleic acid in samples may be either competitive or noncompetitive, and may be either quantitative or non-quantitative. Noncompetitive immunoassays are assays in which antigen is directly detected and, in some instances the amount of antigen directly measured. In a “sandwich” assay, for example, the anti-cyclovirus antibodies can be bound directly to a solid substrate on which they are immobilized. These immobilized antibodies then capture the cyclovirus antigen present in the test sample. Proteins thus immobilized are then bound by a labeling agent, such as a second anti-cyclovirus antigen antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

In competitive assays, cyclovirus antigen present in a sample is detected indirectly by detecting a decrease in a detectable signal associated with a known, added (exogenous) cyclovirus antigen displaced (competed away) from an anti-cyclovirus antigen antibody by the unknown cyclovirus antigen present in a sample. In this manner, such assays can also be adapted to provide for an indirect measurement of the amount of cyclovirus antigen present in the sample. In one competitive assay, a known amount of cyclovirus antigen is added to a sample and the sample is then contacted with an antibody that specifically binds to the cyclovirus antigen. The amount of exogenous cyclovirus antigen bound to the antibody is inversely proportional to the concentration of cyclovirus antigen present in the sample. In a particularly preferred embodiment, the antibody is immobilized on a solid substrate. The amount of cyclovirus antigen bound to the antibody may be determined either by measuring the amount of cyclovirus antigen present in cyclovirus antigen/antibody complex, or alternatively by measuring the amount of remaining uncomplexed protein. The amount of cyclovirus antigen may be detected by providing a labeled cyclovirus antigen.

A hapten inhibition assay is another competitive assay. In this assay the known cyclovirus antigen is immobilized on a solid substrate. A known amount of anti-cyclovirus antigen antibody is added to the sample, and the sample is then contacted with the immobilized cyclovirus antigen. The amount of anti-cyclovirus antigen bound to the known immobilized cyclovirus antigen is inversely proportional to the amount of cyclovirus antigen present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Immunoassays in the competitive binding format can also be used for crossreactivity determinations. For example, an cyclovirus antigen can be immobilized to a solid support. Proteins are added to the assay that compete for binding of the antisera to the immobilized antigen. The ability of the added proteins to compete for binding of the antisera to the immobilized protein is compared to the ability of the cyclovirus antigen to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of an cyclovirus antigen, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the cyclovirus antigen that is required to inhibit 50% of binding, then the second protein is said to specifically bind to the polyclonal antibodies generated to cyclovirus antigen.

Western blot (immunoblot) analysis can be used to detect and quantify the presence of cyclovirus antigen in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the cyclovirus antigen. The anti-cyclovirus antigen antibodies specifically bind to the cyclovirus antigen on the solid support. These antibodies may be directly labeled or alternatively may be subsequently detected using labeled antibodies (e.g., labeled sheep anti-mouse antibodies) that specifically bind to the anti-cyclovirus antigen antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al. (1986) Amer. Clin. Prod. Rev. 5:34-41).

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads, fluorescent dyes (e.g., fluorescein isothiocyanate, Texas red, rhodamine, and the like), radiolabels (e.g., ³H, ¹²⁵I, ³⁵S, ¹⁴C, or ³⁹P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize cyclovirus antigen, or secondary antibodies that recognize anti-cyclovirus antigen.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by the use of electronic detectors such as charge coupled devices (CCDs) or photomultipliers and the like. Similarly, enzymatic labels may be detected by providing the appropriate substrates for the enzyme and detecting the resulting reaction product. Colorimetric or chemiluminescent labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

The present invention provides diagnostic assays to detect cyclovirus, cyclovirus nucleic acids (genome and genes), cyclovirus antibodies in an infected subject, and cyclovirus proteins. In one embodiment, cyclovirus nucleic acids are detected using a nucleic acid amplification-based assay, such as a PCR assay, e.g., in a quantitative assay to determine viral load. In another embodiment, cyclovirus antigens are detected using a serological assay with antibodies (either monoclonal or polyclonal) to antigens encoded by cycloviruses.

In one embodiment of the present invention, the presence of cyclovirus, cyclovirus nucleic acid, or cyclovirus protein in a sample is determined by an immunoassay. Enzyme mediated immunoassays such as immunofluorescence assays (IFA), enzyme linked immunosorbent assays (ELISA) and immunoblotting (western) assays can be readily adapted to accomplish the detection of the cyclovirus or cyclovirus proteins. An ELISA method effective for the detection of the virus can, for example, be as follows: (1) bind an anti-cyclovirus antibody or antigen to a substrate; (2) contact the bound receptor with a fluid or tissue sample containing the virus, a viral antigen, or antibodies to the virus; (3) contact the above with an antibody bound to a detectable moiety (e.g., horseradish peroxidase enzyme or alkaline phosphatase enzyme); (4) contact the above with the substrate for the enzyme; (5) contact the above with a color reagent; (6) observe color change. The above method can be readily modified to detect presence of an anti-cyclovirus antibody in the sample or a specific cyclovirus protein as well as the virus.

Another immunologic technique that can be useful in the detection of cycloviruses is the competitive inhibition assay, utilizing monoclonal antibodies (MABs) specifically reactive with the virus. Briefly, serum or other body fluids from the subject is reacted with an antibody bound to a substrate (e.g., an ELISA 96-well plate). Excess serum is thoroughly washed away. A labeled (enzyme-linked, fluorescent, radioactive, etc.) monoclonal antibody is then reacted with the previously reacted cyclovirus-antibody complex. The amount of inhibition of monoclonal antibody binding is measured relative to a control. MABs can also be used for detection directly in samples by IFA for MABs specifically reactive for the antibody-virus complex.

Alternatively, an cyclovirus antigen and/or a patient's antibodies to the virus can be detected utilizing a capture assay. Briefly, to detect antibodies to cyclovirus in a patient sample, antibodies to the patient's immunoglobulin, e.g., anti-IgG (or IgM) are bound to a solid phase substrate and used to capture the patient's immunoglobulin from serum. An cyclovirus, or reactive fragments of an cyclovirus, can then be contacted with the solid phase followed by addition of a labeled antibody. The amount of patient cyclovirus specific antibody can then be quantitated by the amount of labeled antibody binding.

Additionally, a micro-agglutination test can also be used to detect the presence of cyclovirus in test samples (see e.g., Constantine and Wreghitt (1991) J Med Microbiol. 34(1):29-31). Briefly, latex beads are coated with an antibody and mixed with a test sample, such that cyclovirus in the tissue or body fluids that is specifically reactive with the antibody crosslink with the receptor, causing agglutination. The agglutinated antibody-virus complexes form a precipitate, visible with the naked eye or by spectrophotometer. Other assays include serologic assays, in which the relative concentrations of IgG and IgM are measured.

In the diagnostic methods described above, the sample can be taken directly from the patient or in a partially purified form. The antibody specific for a particular cyclovirus (the primary reaction) reacts by binding to the virus. Thereafter, a secondary reaction with an antibody bound to, or labeled with, a detectable moiety can be added to enhance the detection of the primary reaction. Generally, in the secondary reaction, an antibody or other ligand which is reactive, either specifically or nonspecifically with a different binding site (epitope) of the virus will be selected for its ability to react with multiple sites on the complex of antibody and virus. Thus, for example, several molecules of the antibody in the secondary reaction can react with each complex formed by the primary reaction, making the primary reaction more detectable.

The detectable moiety can allow visual detection of a precipitate or a color change, visual detection by microscopy, or automated detection by spectrometry, radiometric measurement or the like. Examples of detectable moieties include fluorescein and rhodamine (for fluorescence microscopy), horseradish peroxidase (for either light or electron microscopy and biochemical detection), biotin-streptavidin (for light or electron microscopy) and alkaline phosphatase (for biochemical detection by color change). The detection methods and moieties used can be selected, for example, from the list above or other suitable examples by the standard criteria applied to such selections (see e.g., Harlow and Lane (1988) Antibodies: A Laboratory Manual, Cold Spring Harbor Press).

As described herein, an cyclovirus infection may also, or alternatively, be detected based on the level of an cyclovirus RNA or DNA in a biological sample. Primers from cyclovirus sequences can be used for detection of cyclovirus, diagnosis, and determination of cyclovirus viral load. Any suitable primer can be used to detect the genome, nucleic acid sub-sequence, ORF, or protein of choice, using, e.g., methods described in US 20030104009. For example, the subject nucleic acid compositions can be used as single- or double-stranded probes or primers for the detection of cyclovirus mRNA or cDNA generated from such mRNA, as obtained may be present in a biological sample (e.g., extracts of human cells). The cyclovirus polynucleotides of the invention can also be used to generate additional copies of the polynucleotides, to generate antisense oligonucleotides, and as triple-strand forming oligonucleotides. For example, two oligonucleotide primers may be employed in a polymerase chain reaction (PCR) based assay to amplify a portion of cyclovirus cDNA derived from a biological sample, wherein at least one of the oligonucleotide primers is specific for (i.e., hybridizes to) the cyclovirus polynucleotide. The amplified cDNA is then separated and detected using techniques well known in the art, such as gel electrophoresis. Similarly, oligonucleotide probes that specifically hybridize to an cyclovirus polynucleotide may be used in a hybridization assay to detect the presence of the cyclovirus polynucleotide in a biological sample.

The polynucleotides of the invention, particularly where used as a probe in a diagnostic assay, can be detectably labeled. Exemplary detectable labels include, but are not limited to, radiolabels, fluorochromes, (e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrho-damine (TAMRA)), radioactive labels, (e.g. ³²P, ³⁵S, and ³H), and the like. The detectable label can involve two stage systems (e.g., biotin-avidin, hapten-anti-hapten antibody, and the like).

The invention also includes solid substrates, such as arrays, comprising any of the polynucleotides described herein. The polynucleotides are immobilized on the arrays using methods known in the art. An array may have one or more different polynucleotides.

Any suitable qualitative or quantitative methods known in the art for detecting a specific cyclovirus nucleic acid (e.g., RNA or DNA) can be used. Cyclovirus nucleic acids can be detected by, for example, in situ hybridization in tissue sections, using methods that detect single base pair differences between a hybridizing nucleic acid (e.g., using the technology described in U.S. Pat. No. 5,846,717), by reverse transcriptase-PCR, or in Northern blots containing poly A⁺ mRNA, and other methods well known in the art. For detection of cyclovirus polynucleotides in blood or blood-derived samples, the use of methods that allow for detection of single base pair mismatches is preferred.

Using the cyclovirus nucleic acid as a basis, nucleic acid probes (e.g., including oligomers of at least about 8 nucleotides or more) can be prepared, either by excision from recombinant polynucleotides or synthetically, which probes hybridize with the cyclovirus nucleic acid, and thus are useful in detection of cyclovirus virus in a sample, and identification of infected individuals, as well as further characterization of the viral genome(s). The probes for cyclovirus polynucleotides (natural or derived) are of a length or have a sequence which allows the detection of unique viral sequences by hybridization. While about 6-8 nucleotides may be useful, longer sequences may be preferred, e.g., sequences of about 10-12 nucleotides, or about 20 nucleotides or more. Preferably, these sequences will derive from regions which lack heterogeneity among cyclovirus viral isolates.

Nucleic acid probes can be prepared using routine methods, including automated oligonucleotide synthetic methods. A complement to any unique portion of the cyclovirus genome may be used, e.g., a portion of the cyclovirus genome that allows for distinguishing cyclovirus from other viruses that may be present in the sample. For use as probes, complete complementarity is desirable, though it may be unnecessary as the length of the fragment is increased.

For use of such probes as diagnostics, the biological sample to be analyzed, such as blood or serum, may be treated, if desired, to extract the nucleic acids contained therein. The resulting nucleic acid from the sample may be subjected to gel electrophoresis or other size separation techniques; alternatively, the nucleic acid sample may be dot blotted without size separation. The probes are usually labeled with a detectable label. Suitable labels, and methods for labeling probes are known in the art, can include, for example, radioactive labels incorporated by nick translation or kinasing, biotin, fluorescent probes, and chemiluminescent probes. The nucleic acids extracted from the sample are then treated with the labeled probe under hybridization conditions of suitable stringencies.

The probes can be made completely complementary to the cyclovirus genome or portion thereof. Therefore, usually high stringency conditions are desirable in order to prevent or at least minimize false positives. However, conditions of high stringency should only be used if the probes are complementary to regions of the viral genome which lack heterogeneity among cyclovirus viral isolates. The stringency of hybridization is determined by a number of factors during hybridization and during the washing procedure, including temperature, ionic strength, length of time, and concentration of formamide. These factors are outlined in, for example, Sambrook et al. (1989) Molecular Cloning; A Laboratory Manual, Second Edition (Cold Spring Harbor Press, Cold Spring Harbor, N.Y.).

Generally, it is expected that the cyclovirus sequences will be present in a biological sample (e.g., blood, cells, and the liked) obtained from an infected individual at relatively low levels, e.g., at approximately 10²-10⁴ cyclovirus sequences per 10⁶ cells. This level may require that amplification techniques be used in hybridization assays. Such techniques are known in the art.

For example, the Enzo Biochemical Corporation “Bio-Bridge” system uses terminal deoxynucleotide transferase to add unmodified 3′-poly-dT-tails to a DNA probe. The poly dT-tailed probe is hybridized to the target nucleotide sequence, and then to a biotin-modified poly-A. PCT Publication No. WO84/03520 and European application no. EP0,124,221A1 describe a DNA hybridization assay in which: (1) analyte is annealed to a single-stranded DNA probe that is complementary to an enzyme-labeled oligonucleotide; and (2) the resulting tailed duplex is hybridized to an enzyme-labeled oligonucleotide. EP0,204,510B1 describes a DNA hybridization assay in which analyte DNA is contacted with a probe that has a tail, such as a poly-dT tail, an amplifier strand that has a sequence that hybridizes to the tail of the probe, such as a poly-A sequence, and which is capable of binding a plurality of labeled strands.

Non-PCR-based, sequence specific DNA amplification techniques can also be used in the invention to detect cyclovirus sequences. An example of such techniques includes, but is not necessarily limited to the Invader assay, see, e.g., Kwiatkowski et al. (1999) Mol. Diagn. 4(4):353-64; U.S. Pat. No. 5,846,717.

A particularly desirable technique may first involve amplification of the target cyclovirus sequences in sera approximately 10,000 fold, e.g., to approximately 10 sequences/mL. This may be accomplished, for example, by the polymerase chain reactions (PCR) technique described in Mullis, U.S. Pat. No. 4,683,195, and Mullis et al. U.S. Pat. No. 4,683,202. Other amplification methods are well known in the art.

The probes, or alternatively nucleic acid from the samples, may be provided in solution for such assays, or may be affixed to a support (e.g., solid or semi-solid support). Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, and Protein A beads.

In one embodiment, the probe (or sample nucleic acid) is provided on an array for detection. Arrays can be created by, for example, spotting polynucleotide probes onto a substrate (e.g., glass, nitrocellulose, and the like) in a two-dimensional matrix or array. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Techniques for constructing arrays and methods of using these arrays are described in EP0,799,897; WO 97/29212; WO 97/27317; EP0,785,280; WO 97/02357; U.S. Pat. No. 5,593,839; U.S. Pat. No. 5,578,832; EP 728 520; U.S. Pat. No. 5,599,695; EP0,721,016; U.S. Pat. No. 5,556,752; WO 95/22058; and U.S. Pat. No. 5,631,734. Arrays are particularly useful where, for example, a single sample is to be analyzed for the presence of two or more nucleic acid target regions, as the probes for each of the target regions, as well as controls (both positive and negative) can be provided on a single array. Arrays thus facilitate rapid and convenience analysis.

The invention further provides diagnostic reagents and kits comprising one or more such reagents for use in a variety of diagnostic assays, including for example, immunoassays such as ELISA and “sandwich”-type immunoassays, as well as nucleic acid assay, e.g., PCR assays. In a related embodiment, the assay is performed in a flow-through or strip test format, wherein the binding agent is immobilized on a membrane, such as nitrocellulose. Such kits may preferably include at least a first peptide, or a first antibody or antigen binding fragment of the invention, a functional fragment thereof, or a cocktail thereof, or a first oligo pair, and means for signal generation. The kit's components may be pre-attached to a solid support, or may be applied to the surface of a solid support when the kit is used. The signal generating means may come pre-associated with an antibody or nucleic acid of the invention or may require combination with one or more components, e.g., buffers, nucleic acids, antibody-enzyme conjugates, enzyme substrates, or the like, prior to use.

Kits may also include additional reagents, e.g., blocking reagents for reducing nonspecific binding to the solid phase surface, washing reagents, enzyme substrates, enzymes, and the like. The solid phase surface may be in the form of microtiter plates, microspheres, or other materials suitable for immobilizing nucleic acids, proteins, peptides, or polypeptides. An enzyme that catalyzes the formation of a chemiluminescent or chromogenic product or the reduction of a chemiluminescent or chromogenic substrate is one such component of the signal generating means. Such enzymes are well known in the art. Where a radiolabel, chromogenic, fluorigenic, or other type of detectable label or detecting means is included within the kit, the labeling agent may be provided either in the same container as the diagnostic or therapeutic composition itself, or may alternatively be placed in a second distinct container means into which this second composition may be placed and suitably aliquoted. Alternatively, the detection reagent and the label may be prepared in a single container means, and in most cases, the kit will also typically include a means for containing the vial(s) in close confinement for commercial sale and/or convenient packaging and delivery.

Assays for modulators of cycloviruses are also contemplated in the present invention. Modulation of an cyclovirus, and corresponding modulation of the cell cycle or proliferation, can be assessed using a variety of in vitro and in vivo assays, including cell-based models. Such assays can be used to test for inhibitors and activators of cycloviruses. Modulators of cycloviruses are tested using either recombinant or naturally occurring protein of choice.

Measurement of modulation of an cyclovirus or a cell expressing cyclovirus, either recombinant or naturally occurring, can be performed using a variety of assays, in vitro, in vivo, and ex vivo, as described herein. A suitable physical, chemical or phenotypic change that affects activity, e.g., enzymatic activity, cell surface marker expression, viral replication and proliferation can be used to assess the influence of a test compound on the polypeptide of this invention. When the functional effects are determined using intact cells or animals, one can also measure a variety of effects.

Assays to identify compounds with cyclovirus modulating activity can be performed in vitro. Such assays can use full length cyclovirus or a variant thereof, or a mutant thereof, or a fragment thereof, such as a RING domain. Purified recombinant or naturally occurring protein can be used in the in vitro methods of the invention. In addition to purified cyclovirus, the recombinant or naturally occurring protein can be part of a cellular lysate or a cell membrane. As described below, the binding assay can be either solid state or soluble. Preferably, the protein or membrane is bound to a solid support, either covalently or non-covalently. Often, the in vitro assays of the invention are substrate or ligand binding or affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein.

In one embodiment, a high throughput binding assay is performed in which the protein or a fragment thereof is contacted with a potential modulator and incubated for a suitable amount of time. In one embodiment, the potential modulator is bound to a solid support, and the protein is added. In another embodiment, the protein is bound to a solid support. A wide variety of modulators can be used, as described below, including small organic molecules, peptides, antibodies, etc. A wide variety of assays can be used to identify cyclovirus-modulator binding, including labeled protein-protein binding assays, electrophoretic mobility shifts, immunoassays, enzymatic assays, and the like. In some cases, the binding of the candidate modulator is determined through the use of competitive binding assays, where interference with binding of a known ligand or substrate is measured in the presence of a potential modulator. Either the modulator or the known ligand or substrate is bound first, and then the competitor is added. After the protein is washed, interference with binding, either of the potential modulator or of the known ligand or substrate, is determined. Often, either the potential modulator or the known ligand or substrate is labeled.

In another embodiment, the cyclovirus is expressed in a cell, and functional, e.g., physical and chemical or phenotypic, changes are assayed to identify modulators of the cell cycle. Any suitable functional effect can be measured, as described herein. The cyclovirus can be naturally occurring or recombinant. Also, fragments of the cyclovirus or chimeric proteins can be used in cell based assays. In addition, point mutants in essential residues required by the catalytic site can be used in these assays.

The compounds tested as modulators of cyclovirus can be any small organic molecule, or a biological entity, such as a protein, e.g., an antibody or peptide, a sugar, a nucleic acid, e.g., an antisense oligonucleotide or a ribozyme or RNAi, or a lipid. Alternatively, modulators can be genetically altered versions of an cyclovirus. Typically, test compounds will be small organic molecules, peptides, circular peptides, RNAi, antisense molecules, ribozymes, and lipids.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), and Fluka Chemika-Biochemica Analytika (Buchs Switzerland).

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka (1991) Int. J. Pept. Prot. Res. 37:487-493; and Houghton et al. (1991) Nature 354:84-88). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al. (1993) Proc. Nat. Acad. Sci. USA 90:6909-6913), vinylogous polypeptides (Hagihara et al. (1992) J. Amer. Chem. Soc. 114:6568), nonpeptidal peptidomimetics with glucose scaffolding (Hirschmann et al. (1992) J. Amer. Chem. Soc. 114:9217-9218), analogous organic syntheses of small compound libraries (Chen et al., J. Amer. Chem. Soc. 116:2661 (1994)), oligocarbamates (Cho et al. (1993) Science 261:1303), and/or peptidyl phosphonates (Campbell et al. (1994) J. Org. Chem. 59:658), nucleic acid libraries (see Ausubel, Berger and Sambrook, all supra), peptide nucleic acid libraries (see, e.g., U.S. Pat. No. 5,539,083), antibody libraries (see, e.g., Vaughn et al. (1996) Nature Biotechnology, 14(3):309-314 and PCT Publication No. WO 1997/000271), carbohydrate libraries (see, e.g., Liang et al. (1996) Science, 274:1520-1522 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In one embodiment the invention, soluble assays using an cyclovirus, or a cell or tissue expressing an cyclovirus, either naturally occurring or recombinant are provided. In another embodiment, the invention provides solid phase based in vitro assays in a high throughput format, where the cyclovirus is attached to a solid phase. Any one of the assays described herein can be adapted for high throughput screening.

In the high throughput assays of the invention, either soluble or solid state, it is possible to screen up to several thousand different modulators or ligands in a single day. This methodology can be used for cyclovirus in vitro, or for cell-based or membrane-based assays comprising an cyclovirus. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage. A tag for covalent or non-covalent binding can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-glycine sequences of between about 5 and 200 amino acids. Such flexible linkers are known to persons of skill in the art. For example, poly(ethelyne glycol) linkers are available from Shearwater Polymers, Inc. (Huntsville, Ala.). These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield (1963) J. Am. Chem. Soc. 85:2149-2154 (describing solid phase synthesis of, e.g., peptides); Geysen et al. (1987) J. Immun. Meth. 102:259-274 (describing synthesis of solid phase components on pins); Frank & Doring (1988) Tetrahedron 44:60316040 (describing synthesis of various peptide sequences on cellulose disks); Fodor et al. (1991) Science, 251:767-777; Sheldon et al. (1993) Clinical Chemistry 39(4):718-719; and Kozal et al. (1996) Nature Medicine 2(7):753759 (all describing arrays of biopolymers fixed to solid substrates). Non-chemical approaches for fixing tag binders to substrates include other common methods, such as heat, cross-linking by UV radiation, and the like.

Pharmaceutical compositions comprise one or more such vaccine compounds and a physiologically acceptable carrier. Vaccines may comprise one or more such compounds and a non-specific immune response enhancer. A non-specific immune response enhancer may be any substance that enhances an immune response to an exogenous antigen. Examples of non-specific immune response enhancers include adjuvants, biodegradable microspheres (e.g., polylactic galactide) and liposomes (into which the compound is incorporated; see, e.g., U.S. Pat. No. 4,235,877). Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Vaccine preparation is generally described in, for example, Powell and Newman, eds., Vaccine Design (the subunit and adjuvant approach), Plenum Press (NY, 1995). Vaccines may be designed to generate antibody immunity and/or cellular immunity such as that arising from CTL or CD4+ T cells.

Pharmaceutical compositions and vaccines within the scope of the present invention may also contain other compounds, which may be biologically active or inactive. For example, one or more immunogenic portions of other antigens may be present, either incorporated into a fusion polypeptide or as a separate compound, within the composition or vaccine. Polypeptides may, but need not, be conjugated to other macromolecules as described, for example, within U.S. Pat. Nos. 4,372,945 and 4,474,757. Pharmaceutical compositions and vaccines may generally be used for prophylactic and therapeutic purposes.

Nucleic acid vaccines encoding a genome, structural protein or non-structural protein or a fragment thereof of cyclovirus can also be used to elicit an immune response to treat or prevent cyclovirus infection. Numerous gene delivery techniques are well known in the art, such as those described by Rolland (1998) Crit. Rev. Therap. Drug Carrier Systems 15:143-198, and references cited therein. Appropriate nucleic acid expression systems contain the necessary DNA sequences for expression in the patient (such as a suitable promoter and terminating signal). In a preferred embodiment, the DNA may be introduced using a viral expression system (e.g., vaccinia, pox virus, retrovirus, or adenovirus), which may involve the use of a non-pathogenic (defective), replication competent virus. Suitable systems are disclosed, for example, in Fisher-Hoch et al. (1989) Proc. Natl. Acad. Sci. USA 86:317-321; Flexner et al. (1989) Ann. N.Y. Acad. Sci. 569:86-103; Flexner et al. (1990) Vaccine 8:17-21; U.S. Pat. Nos. 4,603,112, 4,769,330, 4,777,127 and 5,017,487; WO 89/01973; GB 2,200,651; EP 0,345,242; WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502; Guzman et al. (1993) Circulation 88:2838-2848; and Guzman et al. (1993) Cir. Res. 73:1202-1207. Techniques for incorporating DNA into such expression systems are well known to those of ordinary skill in the art. The DNA may also be “naked,” as described, for example, in Ulmer et al. (1993) Science 259:1745-1749 and reviewed by Cohen (1993) Science 259:1691-1692. The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions owing to diminished or aberrant expression of the protein, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

All citations are herein incorporated by reference by their entireties. The following examples are intended to illustrate but not limit the invention.

### Example 1

### Sample Collections

Stool samples from South Asian children: A total of 107 fecal specimens were collected by the WHO Regional Reference Laboratory for polio eradication at the National Institute of Health in Islamabad, Pakistan, between December 2005 and May 2008: 57 samples from non-polio-infected children with acute flaccid paralysis (AFP), 9 from closely related but healthy contacts, and 41 from clinically healthy children living in the same geographic region. The median age of the children was 3 years (range, 1 month to 15 years).

Stool samples from Nigerian children: Ninety-six stool samples from non-polio-infected children with AFP were collected by the WHO National Polio Laboratory at the University of Maiduguri Teaching Hospital in Maiduguri. Nigeria, during February to April 2007. The median age of these children was 2.5 years (range, 6 months to 12 years).

Stool samples from Tunisian children and adults: A total of 192 stool samples were collected by the WHO Regional Reference Laboratory for Poliomyelitis and Measles, Institut Pasteur de Tunis, Tunis-Belvedere, Tunisia, from 2005 to 2008, including 94 stool samples from non-polio-infected children with AFP and 82 samples from closely related healthy contact children. Two stool samples from AFP cases and 14 stool samples from healthy contacts were from adults (>15 years). The median age of the cohort was 5 years (range, 6 months to 54 years).

Stool samples from Minnesota patients with gastroenteritis and healthy controls: A total of 247 stool samples from the Minnesota Department of Health were collected from 2004 to 2006, including 107 specimens from clinically healthy donors and 140 specimens from patients with acute gastroenteritis.

Stool samples from African chimpanzees: Forty-four stool samples from individual wild chimpanzees were collected from Central Africa (Tanzania, Cameroon, Rwanda, Uganda, Central African Republic, Republic of the Congo, and Democratic Republic of the Congo). All of the samples were collected from the common chimpanzee (Pan troglodytes) between 2002 and 2007. The geographic sites where the chimpanzee stool samples were collected are shown in Table 3.

Plasma specimens from U.S. blood donors: Ninety-six plasma specimens were collected from unremunerated blood donors in the United States.

Plasma specimens from African bush hunters: A total of 113 plasma specimens were collected from nonsymptomatic bush-hunting African adults (95 specimens) or adults with a nonmalarial fever (18 specimens).

U.S. pork: Thirteen specimens of pork products were purchased from markets in stores in the United States (San Francisco) in September 2008.

Meat products from Pakistan: A total of 57 meat samples were collected from Islamabad, Rawalpindi, and Lahore in Pakistan between August 2008 and March 2009.

Meat products from Nigeria: A total of 147 meat product samples from markets in Maiduguri, Nigeria, were collected during March to April 2009.

All studies were reviewed and approved by the University of California in San Francisco Committee on Human Research.

Nucleotide sequence accession numbers: The sequences of 15 genomes have been deposited in GenBank under accession numbers GQ404844 to GQ404858. Partial Rep gene sequences were deposited in GenBank under accession numbers GQ404858 to GQ404986.

### Example 2

### Molecular Virus Screening and Identification of PK5006 Genome

Prior to random PCR amplification and pyrosequencing viral nucleic acids are partially purified by filtration and nuclease treatment of stool supernatants. The resulting sequence data are analyzed for similarity to known viruses using BLASTx. Inverse nested PCR is used to amplify the genome of CyCV1-PK5006 with the following PCR primers: CV-IR1 (SEQ ID NO:42): 5′-ATTGCTTCGACTGGATGGTCGT-3′, CV-IR2 (SEQ ID NO:43): 5′-AACGACTGGATGGTCGTTCCAC-3′, CV-IF1 (SEQ ID NO:44): 5′-ATTTTCCTTATCCGCATCAACTCC-3′, and CV-IF2 (SEQ ID NO:45): 5′-TACAAACTCAGGTCGCCATTTTG-3′. Full genome sequences of an additional 14 circular genomes are obtained by inverse nested PCR, using primers based on amplified Rep gene fragments

Detection of circoviruses using degenerate primers: Nucleic acids are extracted from stool supernatants and plasma samples using the QIAamp viral RNA kit which extracts both RNA and DNA (Qiagen). DNA is extracted from animal tissue specimens using a QIAamp DNA minikit (Qiagen). Degenerate primers for nested PCR are as follows: CV-F1 (SEQ ID NO:46): 5′-GGNAYNCCNCAYYTNCARGG-3′, CV-R1 (SEQ ID NO:47): 5′-AWCCANCCRTARAARTCRTC-3′, CV-F2 (SEQ ID NO:48): 5′-GGNAYNCCNCAYYTNCARGGNTT-3′, and CV-R2 (SEQ ID NO:49): 5′-TGYTGYTCRTANCCRTCCCACCA-3′. The degenerate primers can be designed on the basis of the consensus sequence from an alignment of replicase (Rep) proteins from CyCV1-PK5006 and 12 representative Circovirus species. Multiple-sequence alignment of the Rep amino acid sequences is performed using ClustalW2, with default settings. PCRs with the degenerate Rep primers are performed with the following cycling profile: 5 min at 95° C.; 40 cycles, with 1 cycle consisting of 1 minute at 95° C., 1 minute at 52° C. (56° C. for the 2nd PCR round), and 1 minute at 72° C.; and a final incubation for 10 minutes at 72° C. Products with a size of approximately 400 bp are purified and sequenced using primer CV-R2. Most of the products are sequenced directly. Amplicons with low concentrations or multiple bands are cloned to obtain high-quality sequence data.

Phylogenetic analysis: Phylogenetic analyses based on aligned amino acid sequences from full-length or partial Rep proteins are generated by the neighbor joining (NJ) method in MEGA 4.1, using amino acid p-distances, with 1,000 bootstrap replicates. Other tree-building methods, maximum parsimony (MEGA) and maximum likelihood (PhyML), are carried out to confirm the NJ tree. The GenBank accession numbers of the Rep sequences from plasmids, viruses, and protists used in the phylogenetic analyses are as follows (shown in brackets): Beak and feather disease virus (BFDV) [AF071878], Canary circovirus (CaCV) [AJ301633], Columbid circovirus (CoCV) [AF252610], Duck circovirus (DuCV) [DQ100076], Goose circovirus (GoCV) [AJ304456], Gull circovirus (GuCV) [DQ845074], Finch circovirus (FiCV) [DQ845075], Raven circovirus (RaCV) [DQ146997], Starling circovirus (StCV) [DQ1729062], Cygnus olor circovirus (SwCV) [EU056310], Porcine circovirus 1 (PCV1) [AY660574], Porcine circovirus 2 (PCV2) [AY424401], Chicken anemia virus (CAV) [M55918], Milk vetch dwarf virus [AB009047], Pepper golden mosaic virus [U57457], Canarypox virus [NC_{—}005309], Giardia intestinalis [AF059664], Bifidobacterium pseudocatenulatum plasmid p4M [NC_{—}003527], and Entamoeba histolytica [XM_{—}643662].

Genome analyses: Putative open reading frames (ORFs) with a coding capacity greater than 100 amino acids are predicted by Vector NT1 Advance 10.3 (Invitrogen). The stem-loop structure is predicted using Mfold (version 3.2).

### Example 3

### A Highly Divergent Circovirus in Human Stool Samples

Viral particles in human stool samples from Pakistani children are enriched by filtration, and contaminating host DNA and RNA are digested by nuclease treatment. Nucleic acids protected within viral capsids were then extracted, amplified using random PCR, and pyrosequenced. The resulting DNA sequences are assembled into contigs, translated, and analyzed by protein similarity search (BLASTx). A contig (1,164 bp) composed of eight sequence reads from the stool sample of a healthy South Asian child (PK5006) is found to have significant similarity to the replicase (Rep) protein of circoviruses (E-value <le-10). Since species in the genus Circovirus have a circular genome, the full viral genome is then amplified by inverse nested PCR, and the amplicon is sequenced by primer walking The virus of the assembled genome was tentatively named “Cyclovirus species 1 strain PK5006” (CyCV1-PK5006) (cyclo means circular in Greek). Sequence alignment of the putative Rep protein of CyCV1-PK5006 with that of known species in the genus Circovirus identified several highly conserved amino acids motifs.

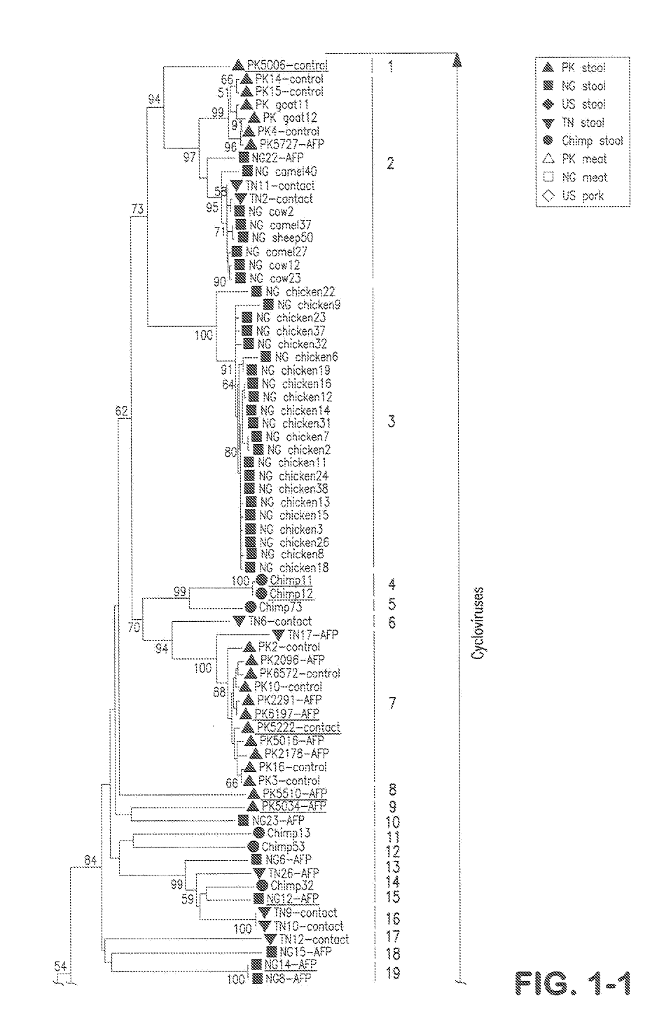

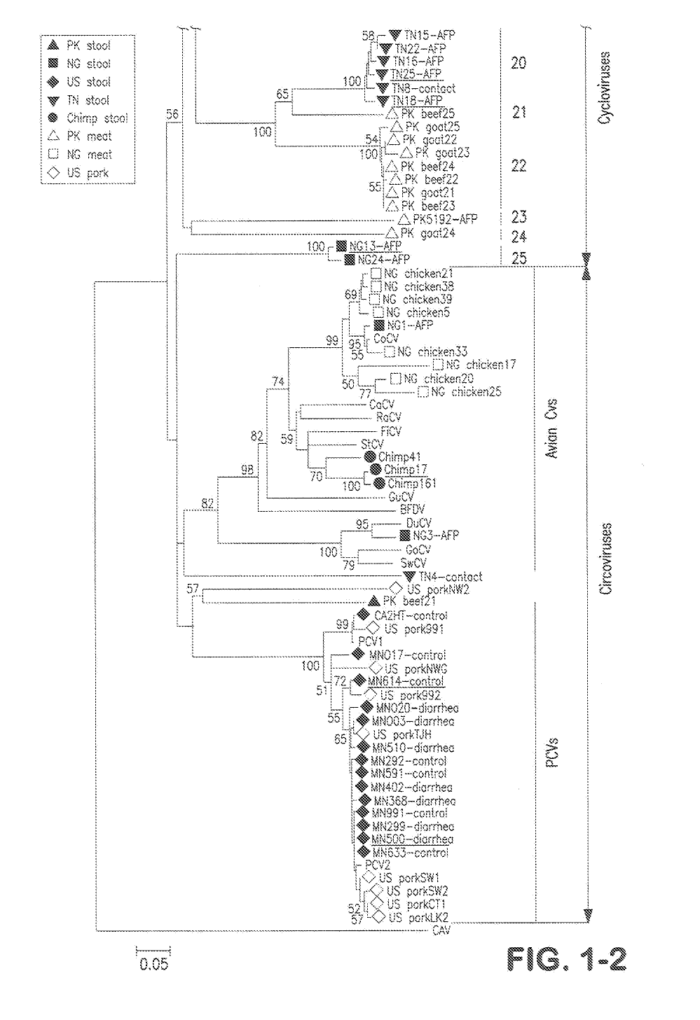

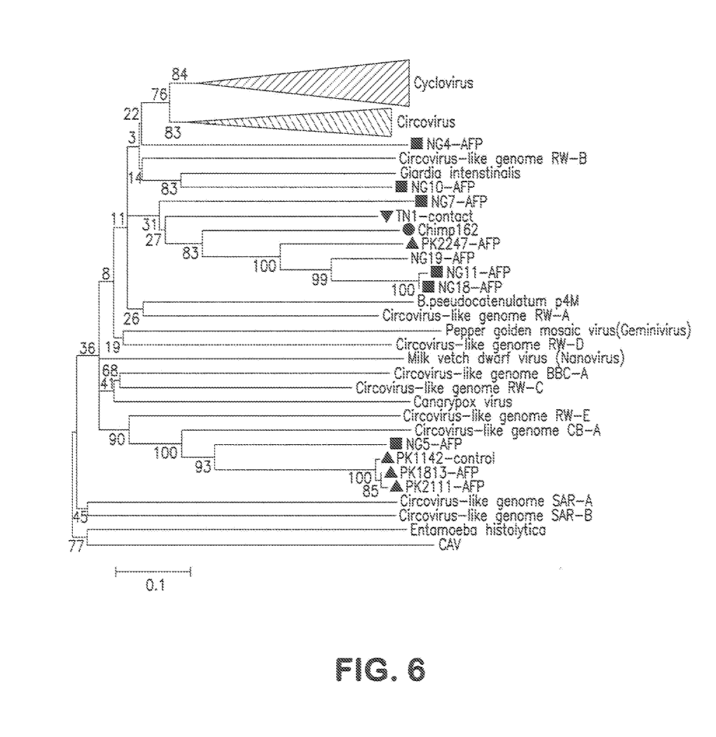

Frequent detection and analysis of circovirus-like Rep sequences in human and animal specimens: To screen for related viruses, the inventors design pan-Rep PCR primers to hybridize to the Rep genes of known avian and porcine circoviruses as well as to the Cyclovirus prototype CyCV1-PK5006. Ten specimen collections of 1,112 samples, including human stool and plasma samples and animal stool and muscle tissue samples are then screened with these primers (Table 1). Rep sequences are detected in 137 samples from all but the two human plasma collections. The approximately 400-bp amplicons are sequenced, and translated amino acids are aligned using the homologous region of the putative Rep-associated protein of CAV as the outgroup. The derived phylogenetic tree is consistent with prior analyses based on the complete Rep protein sequences and on the complete genome of animal circoviruses. A densely populated cluster of Rep sequences (including that of the Cyclovirus prototype genome) is labeled cycloviruses in FIG. 1. Some of the Rep sequences fell outside the Circovirus and Cyclovirus clades, together with the non-Circoviridae Rep proteins from Nanovirus, Geminivirus, Gyrovirus, Canarypox virus, Bifidobacterium pseudocatenulatum plasmid p4M, Giardia intestinalis, and Entamoeba histolytica (see FIG. 6). The possibility that some outlier Rep sequences belong to ingested plant viruses distantly related to nanoviruses and geminiviruses cannot be discounted.

The prevalence of Cyclovirus in human stool samples ranges from 17% in Pakistani children to 0% in U.S. adult stools that exclusively contained PCV1 or PCV2 (Table 1). Rep sequences amplified from the stool samples from two Nigerian children (FIG. 1, NG1-AFP and NG3-AFP) grouped within the avian Circovirus Glade, while sequence amplified from the stool sample of a Tunisian child forms a distinct lineage of circovirus-like sequence (FIG. 1, TN4-contact). Cycloviruses are found in 6 out of 44 stool samples from chimpanzees (14%), and avian circovirus-like sequences are amplified from another 3 chimpanzee stool samples (FIG. 1). No statistical association is found between detection of cyclovirus or circovirus Rep sequences with the occurrence of disease (non-polio AFP in Pakistan or Tunisia or unexplained gastroenteritis in Minnesota).

### Example 4

### Genome Characteristics and Phylogeny of Cycloviruses

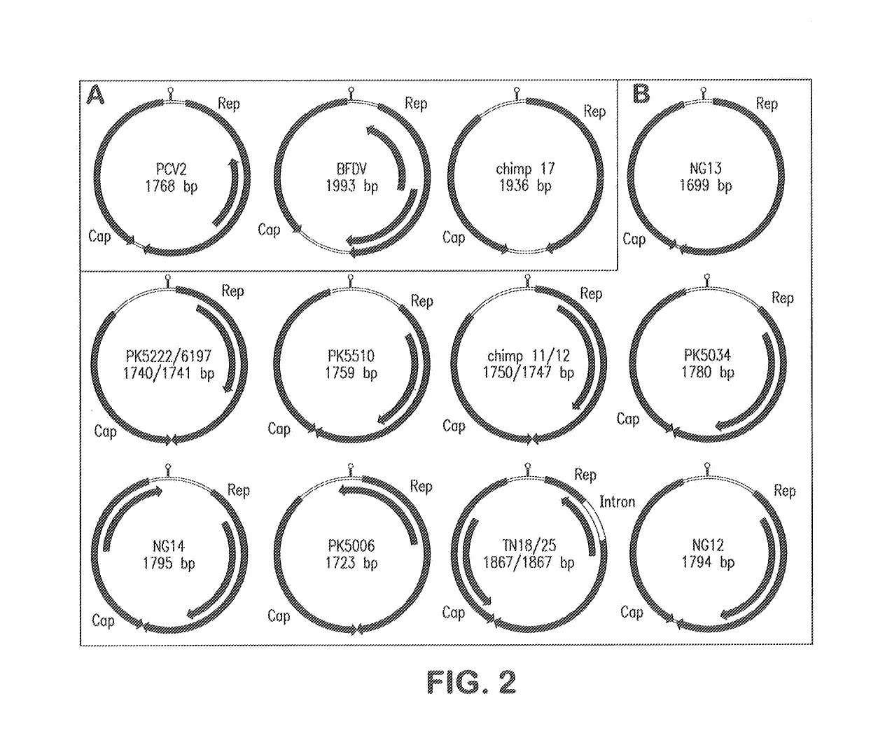

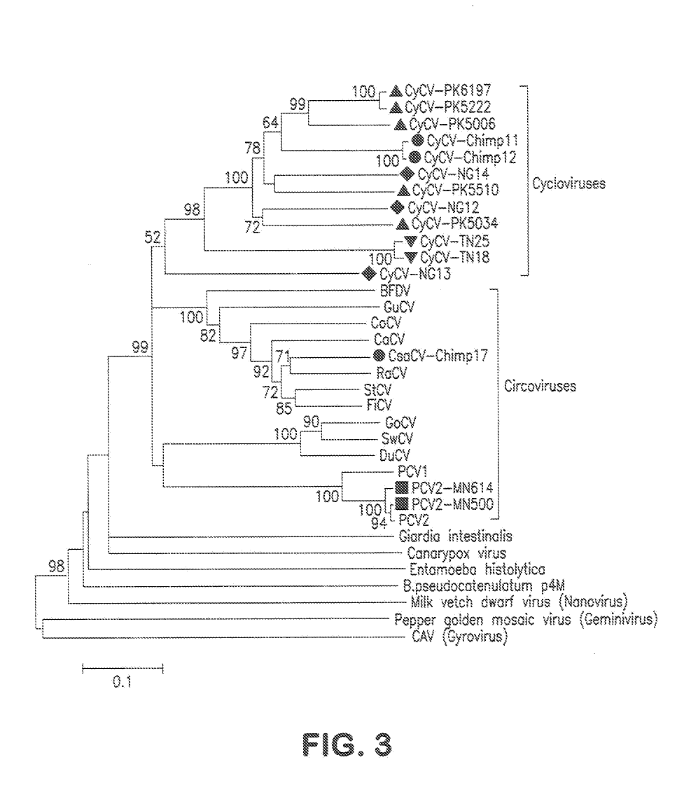

To confirm the presence of diverse cycloviruses and to characterize the genome of this novel group, inverse PCR is used to amplify and sequence complete viral genomes from human and chimpanzee stool samples. Each of the 15 sequenced circular genomes has two main open reading frames arranged in opposite directions, encoding the putative Rep and capsid (Cap) proteins, an arrangement typical of circoviruses (FIG. 2). The complete Rep proteins are used for phylogenetic analysis. The resulting tree confirms the presence of a new Cyclovirus Glade within the Circoviridae, including now 12 genomes (FIG. 3). The ORFs of the Cyclovirus genomes are similar to those of circoviruses but with some distinctive features (FIG. 2). On average, cycloviruses have smaller genomes (average, 1,772 bp; range, 1,699 to 18,67 bp) than circoviruses do (average, 1,902 bp; range, 1,759 to 2,063 bp), encoding relatively smaller Rep and Cap proteins (Tables 2-1 and 2-2). NG13 had the smallest genome size of any reported virus (1,699 bp).

The 3′ intergenic regions between the stop codons of the two major ORFs are either absent or only a few base pairs long in cycloviruses, while those of circoviruses are significantly larger. The 5′ intergenic regions between the start codons of the two major ORFs of cycloviruses are larger than those of circoviruses (Tables 2-1 and 2-2). The Rep ORFs of the two closely related genomes, TN18 and TN25 (97% nucleotide similarity) are both interrupted by an apparent 171-bp intron with a typical splice donor site (GT) and splice acceptor site (AG) (FIG. 2).

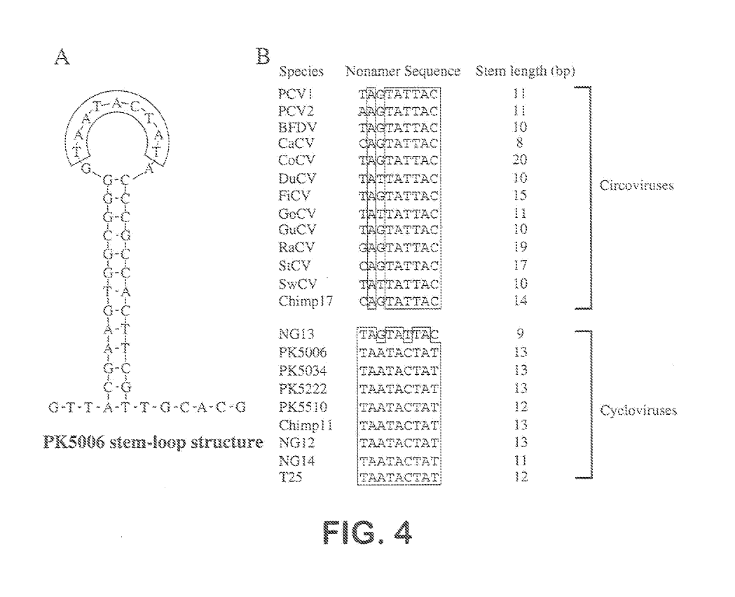

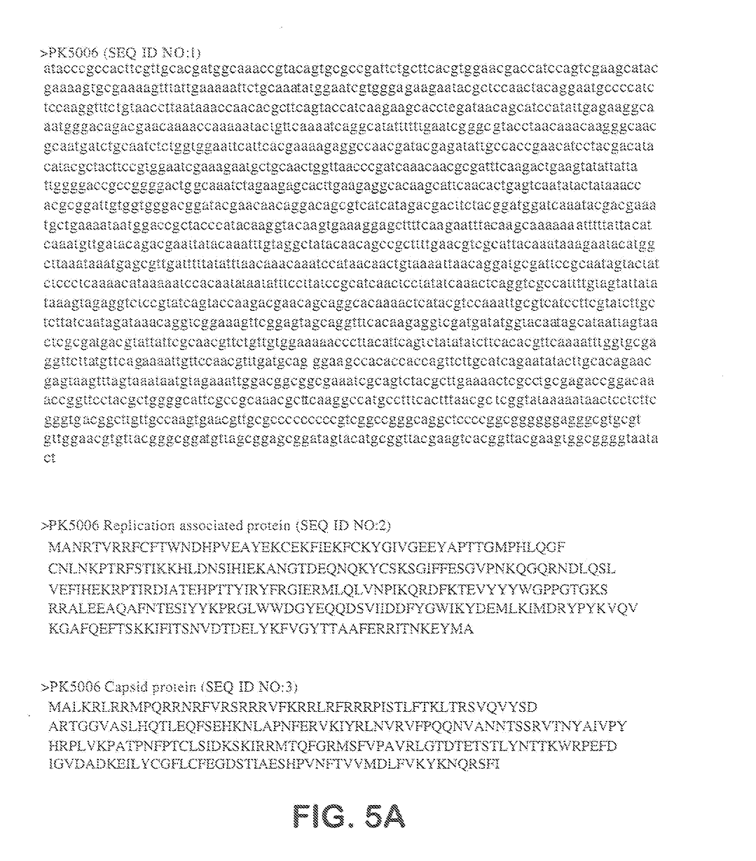

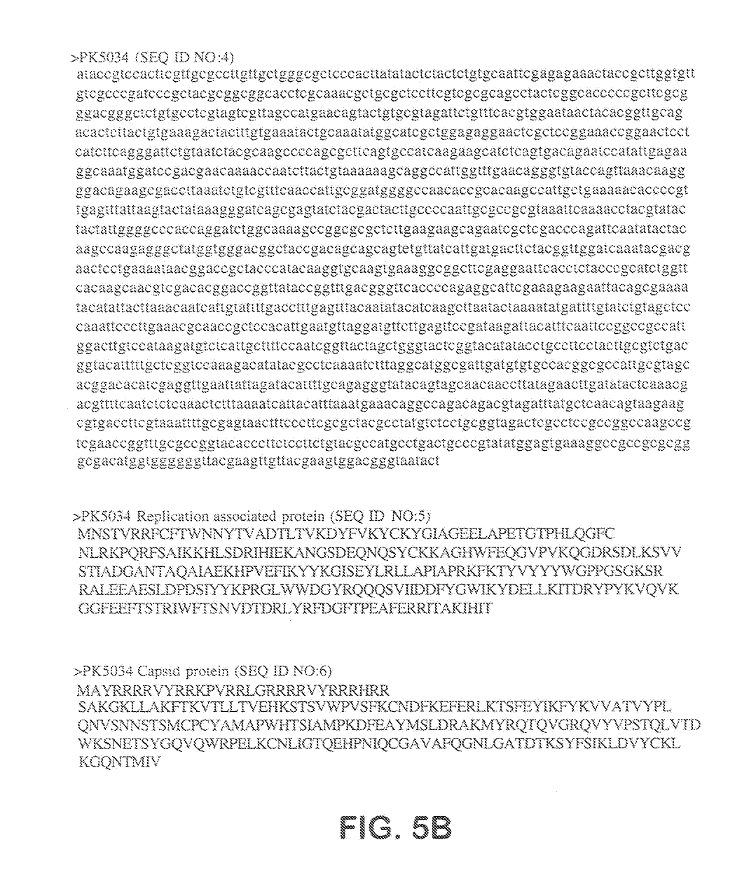

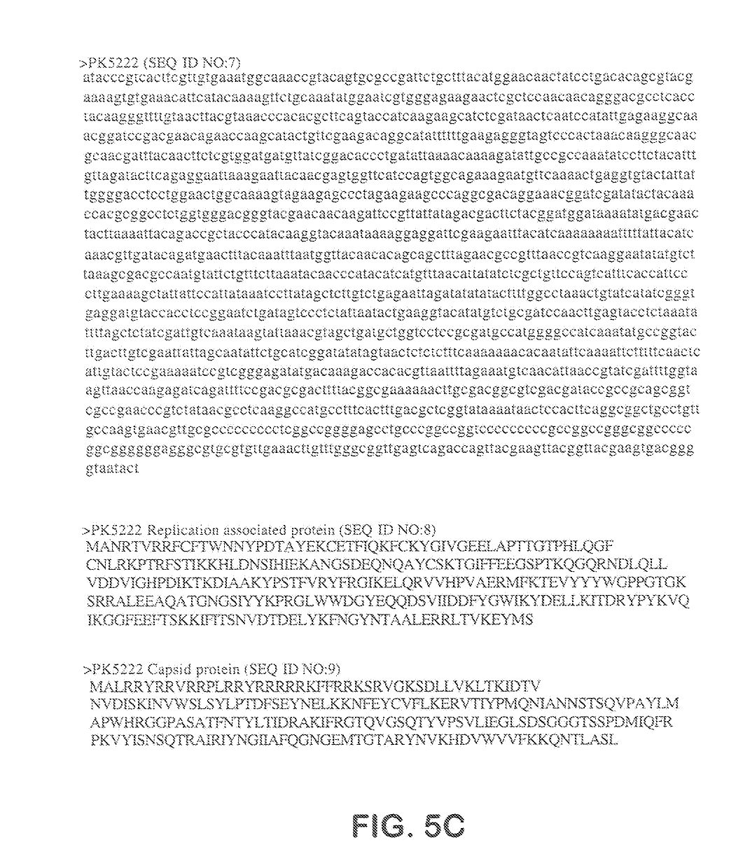

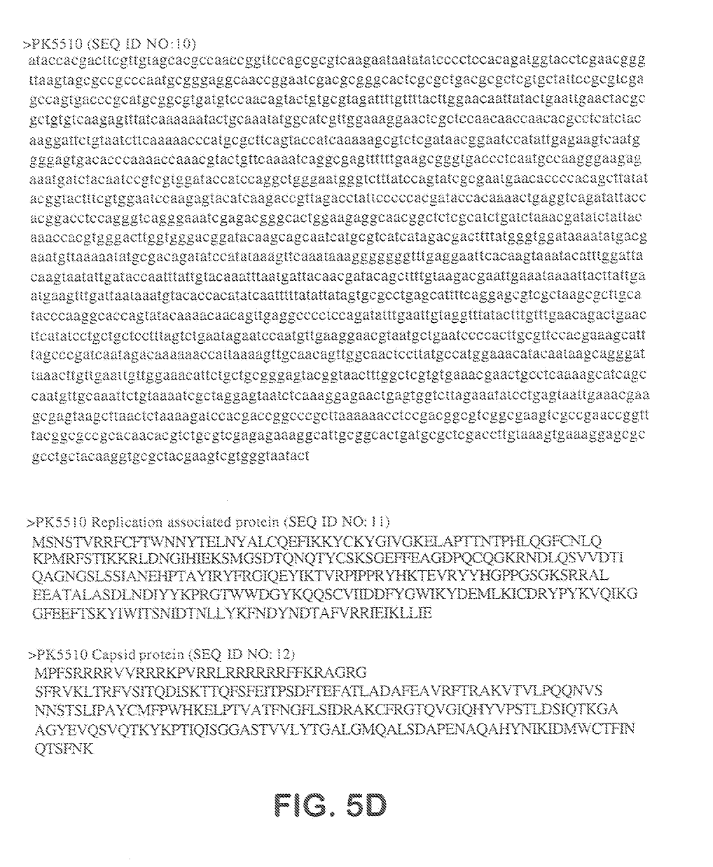

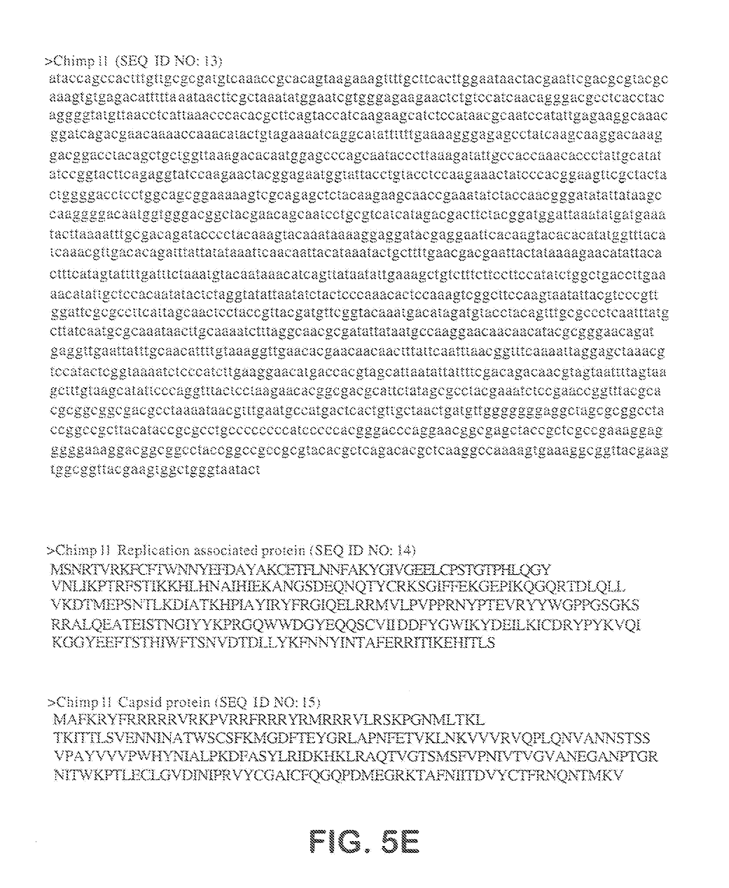

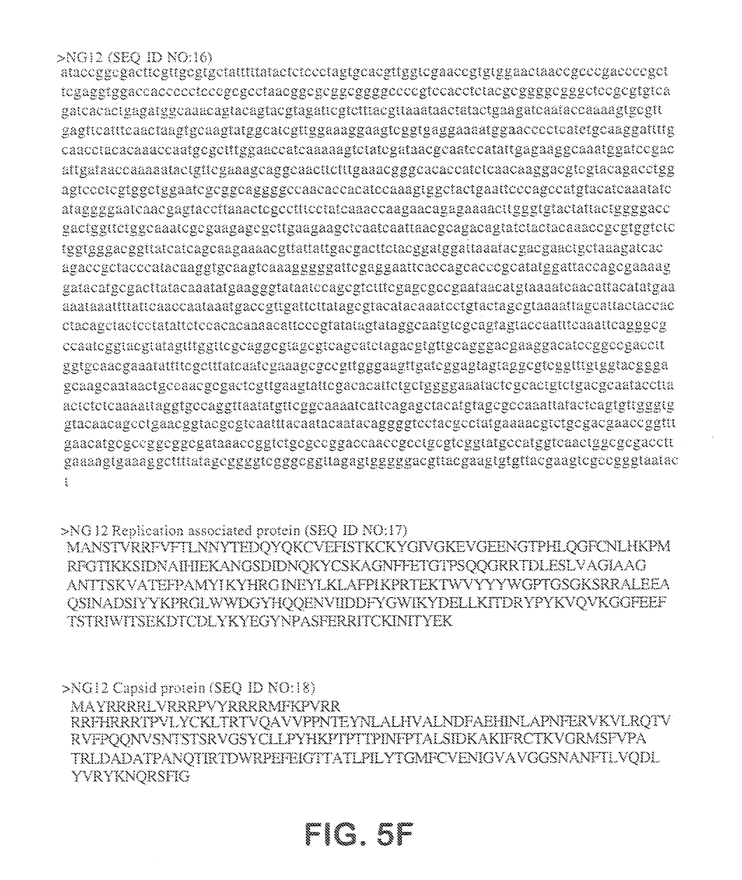

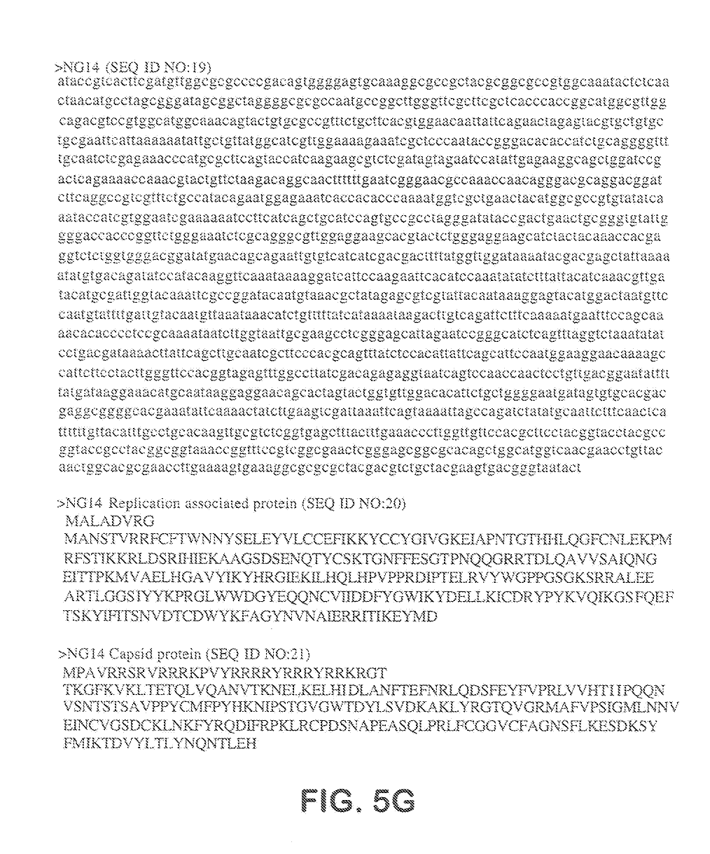

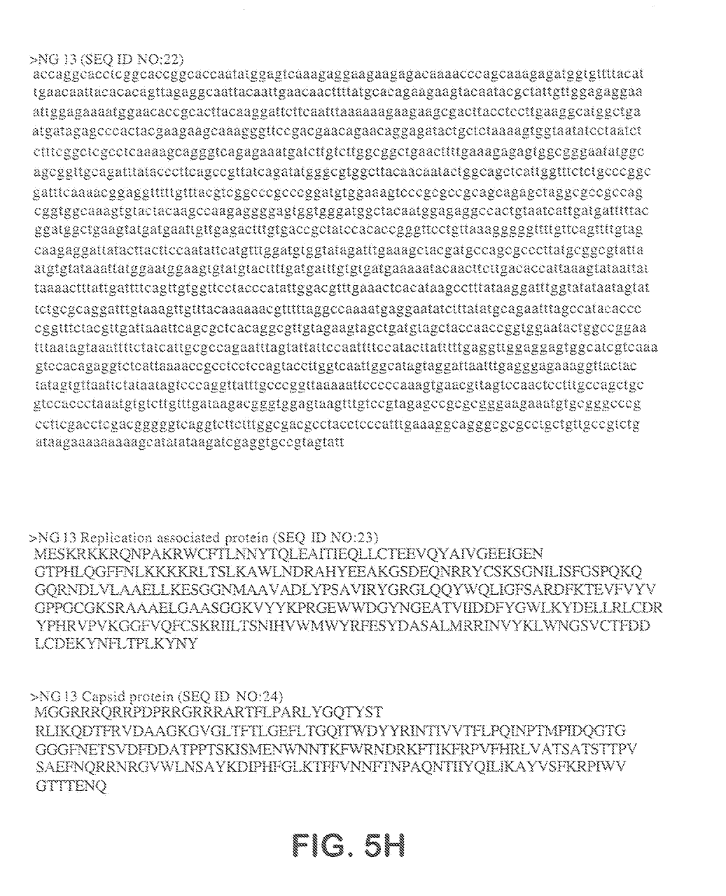

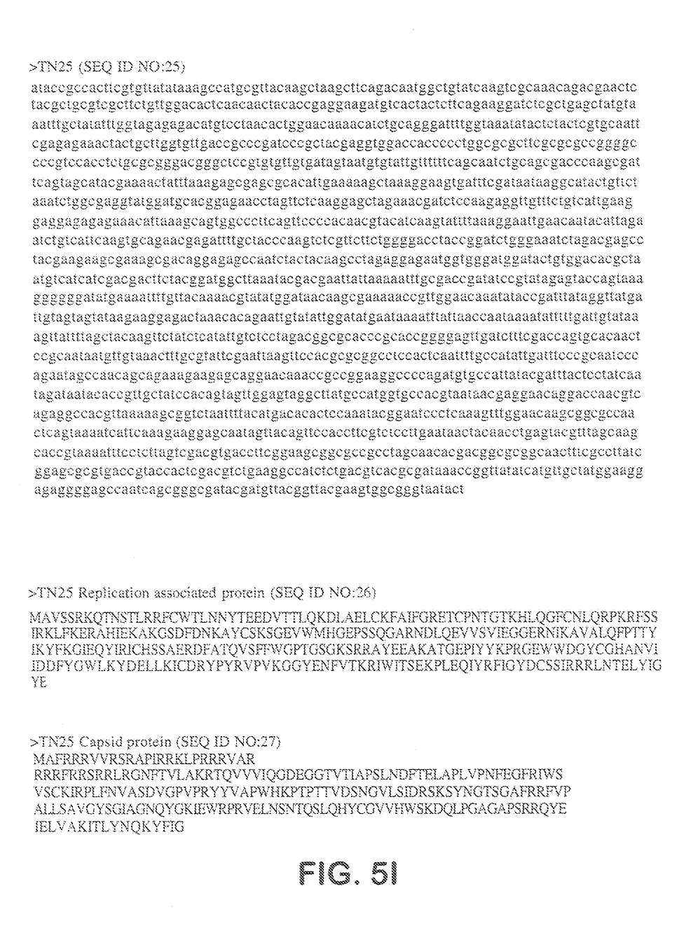

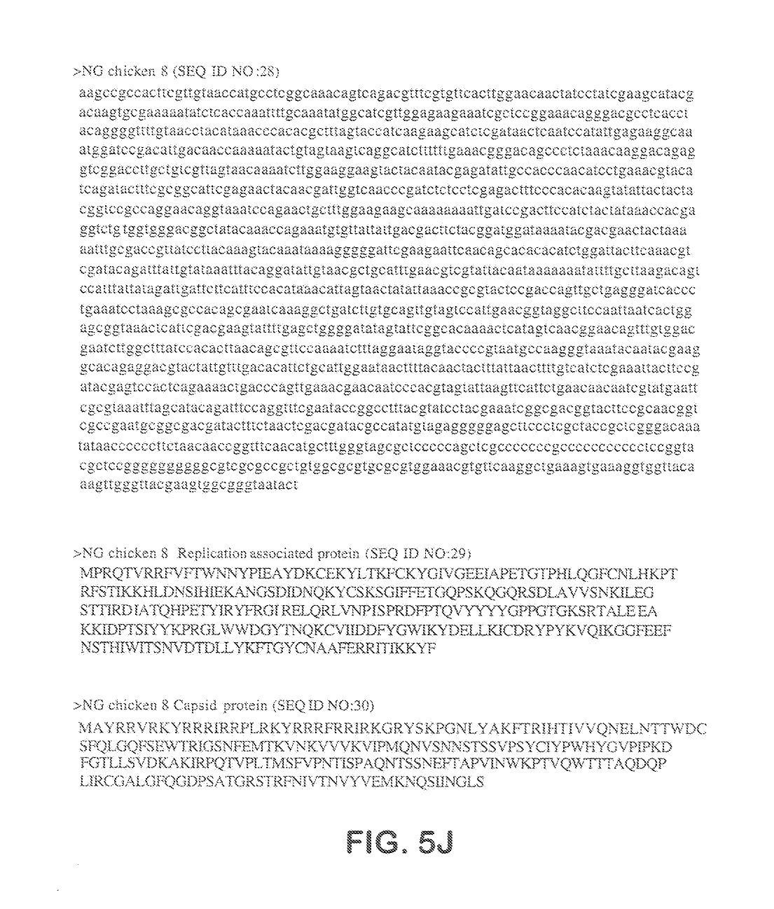

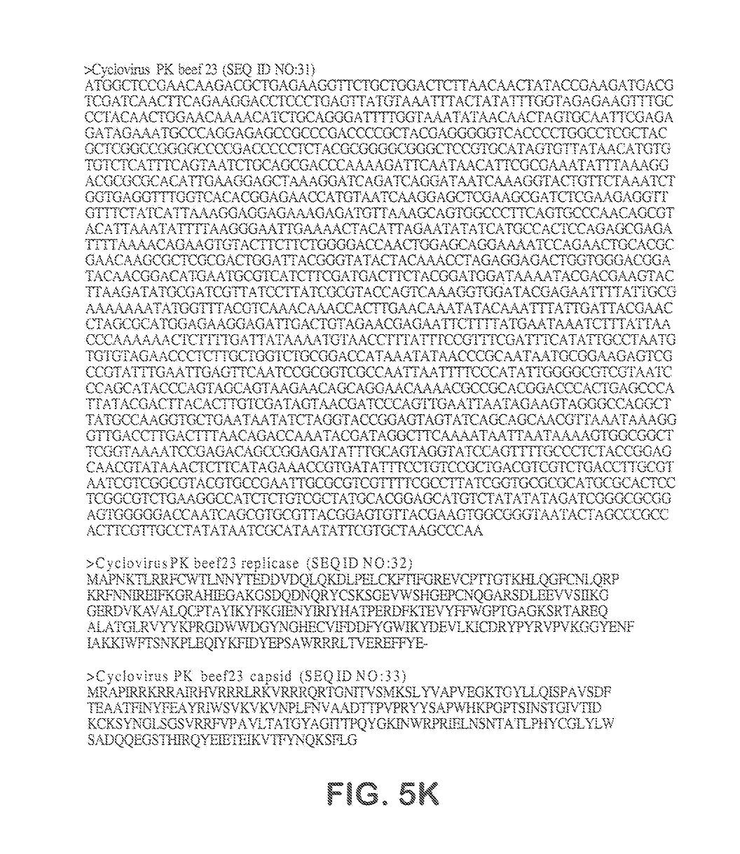

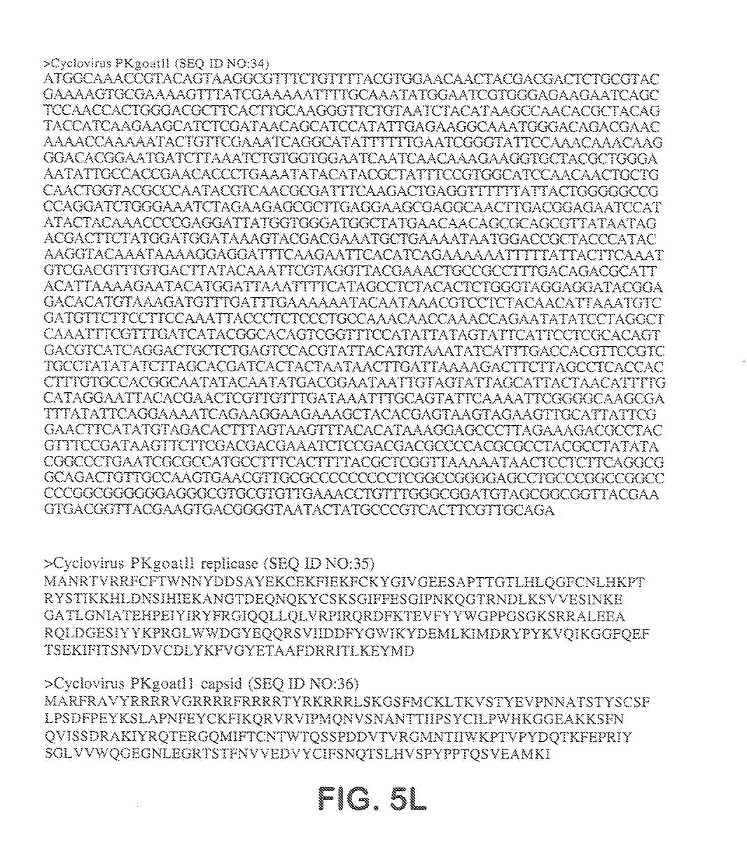

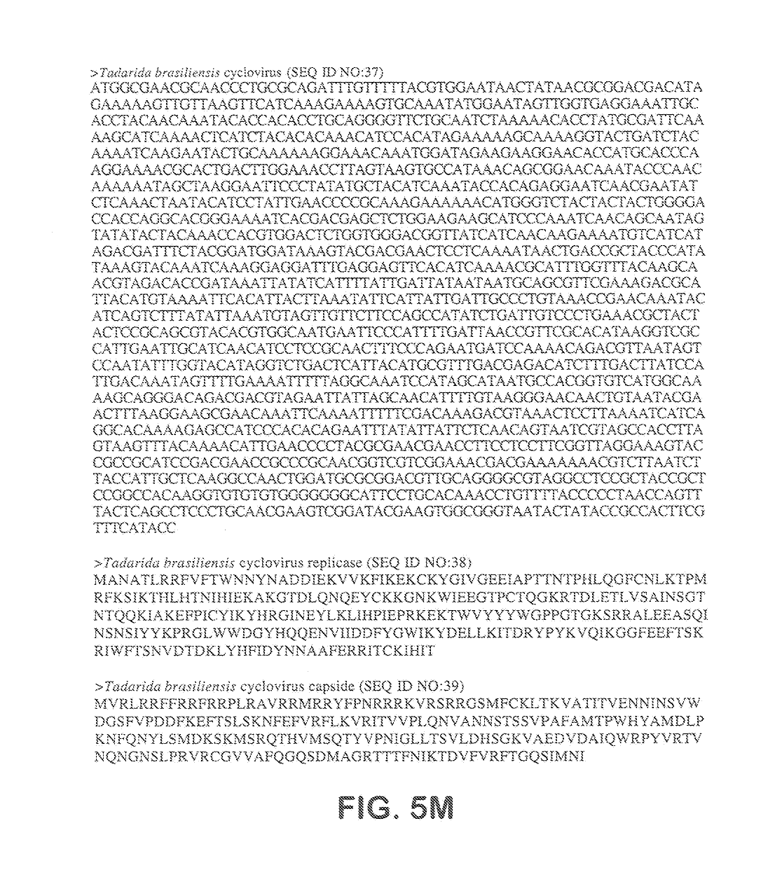

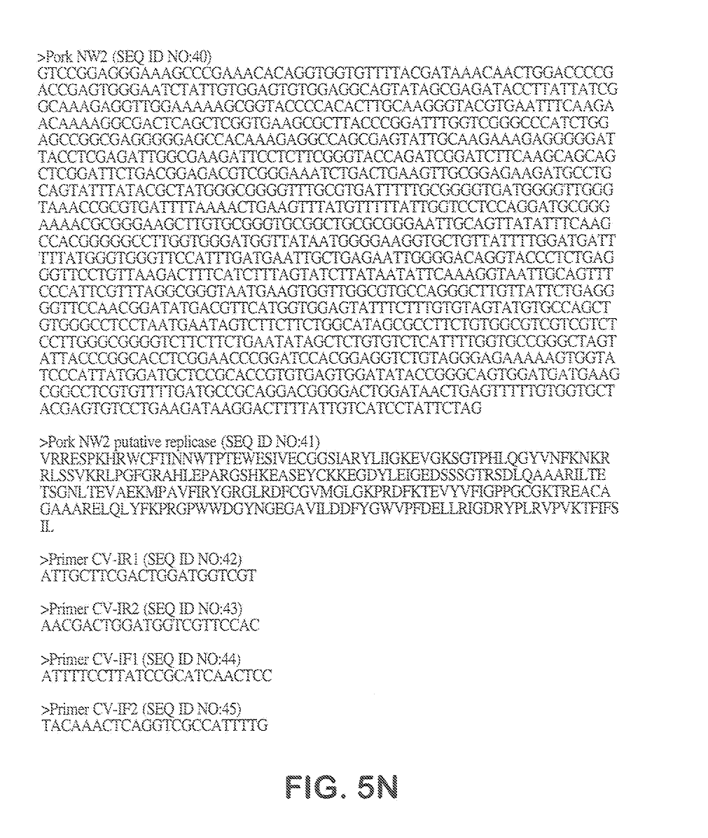

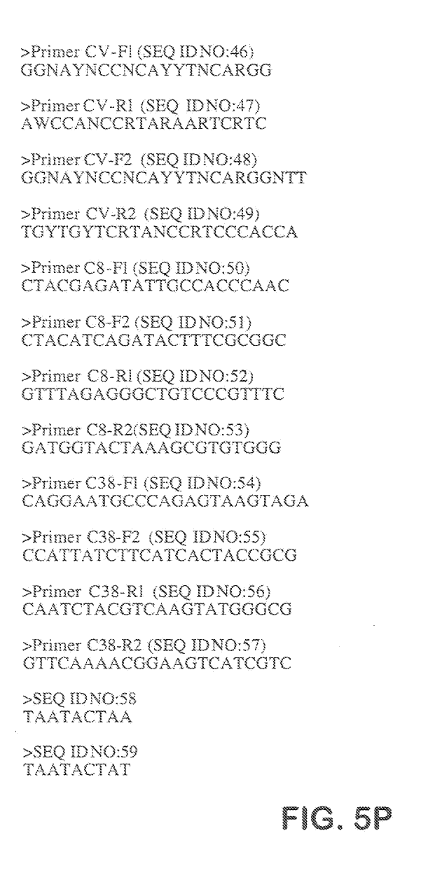

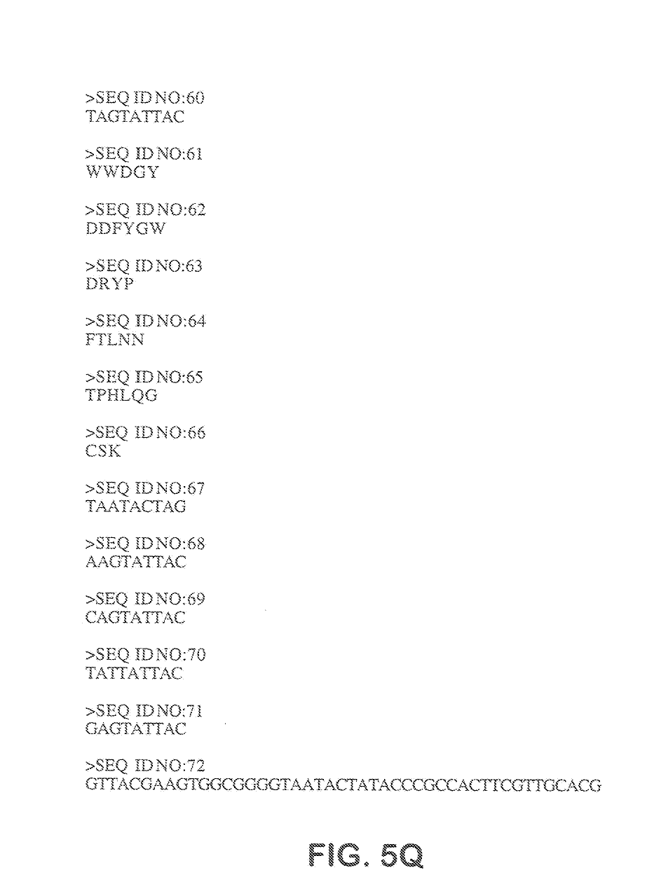

The stem-loop structure with a conserved nonanucleotide motif located at the 5′ intergenic region of circovirus genomes is thought to initiate rolling-cycle replication. A highly conserved stem-loop structure is also found in the 5′ intergenic regions of cycloviruses (FIG. 2 and FIG. 4A). The consensus sequence for the loop nonamer of the circoviruses is 5′-TAGTATTAC-3′ (SEQ ID NO:60), with slight variation among the sequenced genomes (21, 26, 31, 35, 38, 39) (FIG. 4B). A different and conserved loop nonamer sequence 5′-TAATACTAT-3′ (SEQ ID NO:59) is observed for all the cycloviruses except CyCV-NG13, which is a cyclovirus group outlier but carries a typical circovirus nonamer (FIG. 4B). The highly conserved nonamer atop the stem-loop structure is one of the distinct characteristics of the new Cyclovirus genus.

The two genomes derived from human stool samples in the United States (MN614 and MN500) share 99% overall genome nucleotide similarity with PCV2. The Chimpl7 genome, from a chimpanzee stool sample, grouped with the raven circovirus RaCV, shares 79% amino acid similarity to its Rep protein. The present invention provides this virus as “Chimpanzee Stool avian-like circovirus-chimp17” (CsaCV-chimp17). No suitably located ATG is identified for either ORF of CsaCV-chimp17. Considering the common usage of alternative start codons in avian circoviruses, such as TCT, GTG, and ATA, CTG is considered the most likely candidate for a start codon in the genome, producing ORFs of expected lengths.

The average amino acid similarity among cyclovirus Rep proteins is 59% (range, 42 to 80%), and the value for circovirus Rep proteins is 56% (range, 40 to 87%), reflecting a comparable range of viral diversity within both genera (see Table 4). For the capsid protein, the average amino acid similarity is 29% (range, 11 to 56%) for cycloviruses and 34% (range, 18 to 76%) for circoviruses (see Table 4). An amino acid alignment shows that cycloviruses also possess some of the highly conserved Rep amino acid motifs typical of circoviruses, including WWDGY (SEQ ID NO:61), DDFYGW (SEQ ID NO:62), and DRYP (SEQ ID NO:63). Motifs associated with rolling-circle replication [FTLNN (SEQ ID NO:64), TPHLQG (SEQ ID NO:65), and CSK (SEQ ID NO:66)] and deoxynucleoside triphosphate (dNTP) binding (G-GSK) are also identified, with some alterations. The N-terminal region of the cyclovirus Cap proteins is highly basic and arginine-rich, as is typical for circoviruses.

### Example 5

### PCVs Frequently Detected in U.S. Human Stool Samples and Pork Products

All Rep sequences derived from human stool samples from the United States cluster closely with PCVs (FIG. 1). In order to test the possible dietary origin of these PCV sequences, pork specimens purchased from different U.S. stores are tested. Out of 13 U.S. pork products, 9 (70%) are Rep positive, 7 of which cluster with PCV2, 1 with PCV1, and 1 highly divergent sequence (US porkNW2) (FIG. 1). Pork sample US porkNW2 may represent a yet uncharacterized porcine circovirus species. Out of 23 Rep sequences from the U.S. samples, 22, including all 14 from human stool samples and 8 out of 9 from pork specimens, therefore belong to PCV1 or PCV2.

Circovirus-like Rep sequences in consumed meats: The frequent detection of PCVs in U.S. stool samples and U.S. pork products suggests that the cycloviruses found in non-U.S. human stool samples and wild chimpanzee stool samples might similarly originate from the consumption of meat contaminated with cycloviruses. Commonly eaten meat products are acquired from markets in Pakistan and Nigeria and analyzed by pan-Rep PCR (Table 1). Of 204 meat samples tested, 24% are positive, and all amplicons were sequenced. The Rep sequence detection rates differed substantially between countries for the same type of meat. None of 13 chicken samples from Pakistan is positive, while 30 out of 40 (75%) chicken samples from Nigeria are positive. Of the 30 Rep sequences from Nigerian chicken samples, 22 sequences cluster tightly within the Cyclovirus genus, and 8 sequences cluster together in a cluster with pigeon Circovirus (as did the Rep sequence NG1-AFP from the stool sample of a Nigerian child). Of the 26 goat samples from Nigeria, none is positive, while 7 out of 18 (38%) goat specimens from Pakistan are positive for cycloviruses. Of the total 19 Rep sequences obtained from farm mammals (cows, goats, sheep, and camels), only 1 (PK beef21) grouped deeply with the circovirus Glade, while 18 fell within the cyclovirus Glade. The majority (40 out of 49) of Rep sequences obtained from animal tissue therefore belongs to the cyclovirus Glade. Some cyclovirus Rep sequences from different animal species (e.g., cows and goats, even-toed ungulates in the Bovidae family) are very closely related (FIG. 1, species 22).

The ICTV defines different circovirus species based on sequence similarity; genomic sequences having <75% nucleotide identity and <70% identity in the capsid protein qualify as different species. The present invention provides a criterion of <85% amino acid identity in the highly conserved Rep protein region amplified by pan-Rep PCR as the criterion for Cyclovirus species designation by comparing the amino acid identity of the same Rep region among known circovirus species. Using this criterion, 25 species of Cyclovirus are found in human and chimpanzee stool samples and meat samples from farm animals. Of these 25 species, only a single Cyclovirus species (FIG. 1, species 2), represented by 16 out of a total of 88 Rep sequences (18.2%), is found in both human stool and farm animal tissue samples. Four species are specific to chimpanzee stool samples, and another four species were specific to farm animals. Sixteen cyclovirus species are specific to non-U.S. human stool samples. The consumption of meat from infected animals is therefore unlikely to account for the majority of cycloviruses detected in non-U.S. human stool samples.

### Example 6

### Analysis of Cycloviruses Sequences

The present invention provides the frequent detection of viral, circular DNA genomes related to porcine and avian circoviruses in human and chimpanzee stool samples and genetically characterize a previously unrecognized genus in the family Circoviridae. These viruses are both widely dispersed (Tunisia, Pakistan, and Nigeria) and highly prevalent (7 to 17% of children's stool samples.)

Cycloviruses are not closely related phylogenetically to the recently described circular DNA viruses chimpanzee stool-associated circular viruses (ChiSCV) found in chimpanzee stool samples or the circular ssDNA viruses in aquatic environments, nor is their genome organization related to human or animal anelloviruses (e.g. torque teno virus [TTV]).

PCVs are frequently detected in stool samples from adults in the United States (5%), and store-bought pork products also frequently contain PCV sequences (70%). These results indicat that detection of PCV DNA in stool may reflect dietary consumption of PCV-infected pork.

Evidence for circovirus infection in mammals other than pigs is equivocal, and studies have been restricted to PCVs. PCV2 DNA in cows with respiratory symptoms and in aborted bovine fetuses has been reported only once. PCV2 is also reported in a colon biopsy specimen from a patient with ulcerative colitis, although contamination with PCV2 from stool is difficult to exclude in this case. No PCV DNA is found by PCR in screening more than 1,000 samples from various tissues of both healthy and immunosuppressed humans and plasma samples from 18 xenotransplantation recipients of pig islet cells. In this study, the results of screening plasma samples from 96 U.S. blood donors and 113 Central African bush hunters via pan-Rep PCR are also negative (Table 1). One study shows that viral protein expression, cytopathic effect, and DNA persistence occurs in human cell lines infected with PCV2, but the virus can not be passed to new cultures. Another study shows the presence of PCV-reactive antibodies, although with somewhat distinctive properties in sera of humans, cows, and mice, while another reported the lack of PCV antibodies in cows and horses.

Avian circovirus-like DNA is found in 3/44 wild chimpanzee stool samples and in 2/96 stool samples from Nigerian children (Table 1). This observation may reflect consumption of infected birds or conceivably contamination of food with bird droppings.

Cycloviruses are found in the muscle tissue of all the species of farm animals tested (goats, sheep, cows, camels, and chickens), suggesting that viral infection occurs in these species. In previous studies, different tissues have been shown to retain small DNA viruses (e.g., parvoviruses) long after primary infection viremia. The detection of cycloviruses and circoviruses in muscle tissue can therefore reflect prior and/or ongoing infection. The detection of closely related cyclovirus Rep sequences in both cows and goats from Pakistan (FIG. 1, species 22) may reflect cross-species transmission.

A wide diversity of cycloviruses is identified in human stool samples collected from children in developing countries. In contrast, in the United States, all Rep sequences obtained from stool samples belong to the PCV Glade. An important distinction between U.S. and non-U.S. human stool samples is the younger age of the non-U.S. donors, which may have impacted host susceptibility to infections or the duration of viral shedding. Exposure to cyclovirus may therefore also occur in the United States but was not detected because of the older age of the subjects.

In total, 17 Cyclovirus species are identified in 395 human stool samples, and 5 Cyclovirus species are found in muscle tissue samples from 204 farm animals, with only a single species found in common in both groups of samples. The meat samples analyzed are acquired from three major cities in Pakistan and one major city in Nigeria, while the children from these countries shedding cycloviruses are geographically more dispersed. The present invention provides that despite the large number of cyclovirus replicase sequences generated, more geographically dispersed sampling of farm animals would have shown greater overlap with human stool-derived cyclovirus sequences. Using the current sampling, the limited overlap between Cyclovirus species found in human stool samples and in meat from farm animals from the same countries does suggest that most of the cycloviruses found in the stool samples of children in Nigeria and Pakistan are not from consumed meat. Possibly, the 16 cycloviruses species found only in human stool samples are transmitted via a fecal-oral route from other infected children, a common pathway for many enteric viral infections. The detection of cycloviruses in 14% of stool samples from chimpanzees (who consume very limited amounts of meat) also argues in favor of transmission within this primate species rather than simply reflecting consumption of infected meats. It is not known whether the viral species found in both human stool samples and tissue samples from farm animals, such as PCVs in the United States and cyclovirus species 2 (FIG. 1, species 2) in Pakistan, Nigeria, and Tunisia, can replicate in their human host. Since transmission of PCV2 from one pig to another through consumption of meat is recently shown, the potential for zoonotic transfer also exists for other circoviruses and cycloviruses.

Given the high prevalence of cyclovirus infections in non-U.S. farm animals, the possibility of cross-species transmission (cyclovirus species 22 in different members of the family Bovidae), the high diversity of cycloviruses in human stool samples, the documented pathogenicity of closely related Circovirus species, and the high rate of mutation and recombination of some ssDNA viruses, the pathogenic potential of cycloviruses in both humans and farm animals can be significant.

### Example 7

### Cyclovirus and Circovirus Isolated from Animals

The present invention provides at least six cyclovirus and four circovirus genomes from the tissues of chickens, goats, cows, and a bat, which are amplified and sequenced using rolling-circle amplification and inverse PCR. A goat cyclovirus is nearly identical to a cyclovirus in a cow. US beef can contain circoviruses >99% similar to porcine PCV2b. Circoviruses in chicken are related to those of pigeons. The close genetic similarity of a subset of cycloviruses and circoviruses replicating in distinct animal species may reflect recent cross-species transmissions.

Members of the Circoviridae family, are non-enveloped, spherical viruses with a single-stranded circular DNA genome of approximately 2 kb, the smallest known autonomously replicating viral genomes. Circoviruses cause a variety of clinical symptoms in birds and pigs including lethargy, lymphoid depletion and immunosuppression. Both circoviruses and cycloviruses have an ambisense genome organization containing two major inversely-arranged open reading frames (ORF) encoding the putative replication-associated (Rep) and capsid protein (Cap). A potential stem-loop structure with a conserved nonanucleotide motif located between 5′-ends of these two ORFs is required to initiate the replication of the viral genome. Cycloviruses are distinguishable from circoviruses by missing one intergenic region, containing a different conserved nonamer sequence atop their stem-loop structure, and by phylogenetically clustering separately from the circoviruses.

To date no circovirus infection has been reported in chicken, and porcine circovirus 1 and 2 (PCV1 and PCV2) are the only two circoviruses reported to infect mammals. Using degenerate PCR primers based on highly conserved amino acid motifs in the Rep proteins, both circovirus and cyclovirus related sequences are recently detected in muscle tissues of animals including chickens, cows, sheep, goats, and camels from Pakistan and Nigeria, but complete genomes were not obtained from these tissues. In this example and following examples, rolling-circle amplification, using the illustra TempliPhi 100 Amplification Kit (GE Health Care) according to a modified protocol optimized for the amplification of viral circular DNA genomes, and inverse PCR are performed to amplify and sequence some of these viruses. The complete genome sequences of eleven circoviruses and cycloviruses from farm animals and a bat are obtained. Extended PCR prevalence search to chicken, pork and beef are carried out from stores stores in California, USA. The US meat samples (chicken, beef, and pork) were collected from California, USA in September 2008, and from October 2009 to July 2010.

### Example 8

### Cyclovirus and Circovirus Isolated from Chicken

The present invention provides that none of the 13 Pakistani but 30 out of the 40 Nigerian chicken muscle tissue samples are PCR positive for the Rep gene. 22 of the 30 Nigerian sequences were closely related and belonged to the cyclovirus genus, while 8 sequences clustered with pigeon circovirus (CoCV). All 21 San Francisco supermarkets bought chicken samples are negative for circovirus-like Rep sequence.

Two chicken cyclovirus and one circovirus circular genomes are then amplified by inverse PCR from the nucleic acid extracted from chicken muscle samples (QIAamp DNA Mini Kit from Qiagen). Nested PCR are used with the following two primer sets designed according to the Rep gene fragments previously amplified with the pan-Rep primers: C8-F1 (SEQ ID NO:50): 5′-CTACGAGATATTGCCACCCAAC-3′, C8-F2 (SEQ ID NO:51): 5′-CTACATCAGATACTTTCGCGGC-3′, C8-R1 (SEQ ID NO:52): 5′-GTTTAGAGGGCTGTCCCGTTTC-3′, C8-R2 (SEQ ID NO:53): 5′-GATGGTACTAAAGCGTGTGGG-3′ for the two cycloviruses and C38-F1 (SEQ ID NO:54): 5′-CAGGAATGCCCAGAGTAAGTAGA-3′, C38-F2 (SEQ ID NO:55): 5′-CCATTATCTTCATCACTACCGCG-3′, C38-R1 (SEQ ID NO:56): 5′-CAATCTACGTCAAGTATGGGCG-3′, C38-R2 (SEQ ID NO:57): 5′-GTTCAAAACGGAAGTCATCGTC-3′ for the circovirus). PCR reactions are carried out with the following cycling profile: 95° C. for 5 minutes, 39 cycles with 95° C. for 1 minute, 55° C. for 1 minute (57° C. for the 2nd PCR round), and 72° C. for 1.5 minutes, and a final incubation for 10 minutes at 72° C. The resulting PCR products (approximately 1.7 kb) are purified and cloned into the pGEM-T Easy Vector (Promega). The nucleotide sequence of the genomes are covered twice and the sequences are deposited in GenBank. The putative ORFs are predicted by Vector NTI (Invitrogen), taking into consideration the organization of other circoviruses. Phylogenetic analyses based on aligned amino acid sequences of the full-length Rep proteins are generated by the neighbor joining method in MEGA 4.1, using amino acid p-distances, with 1,000 bootstrap replicates. The stem-loop structure was predicted using Mfold (version 3.2).

The full-length genomes of two closely related chicken cycloviruses (CyCV-NG chicken 8 and CyCV-NG chicken 15) and that of the chicken circovirus (CV-NG chicken 38) are obtained. Sequence analysis reveals that the cycloviruses were 1760 nucleotides long and circular, and shared 99% nucleotide identity differing at only 10 nucleotides, resulting in one amino acid change in the Rep and the Capsid proteins. The chicken circovirus genome is 2037 nucleotides and is 92% identical to CoCV.

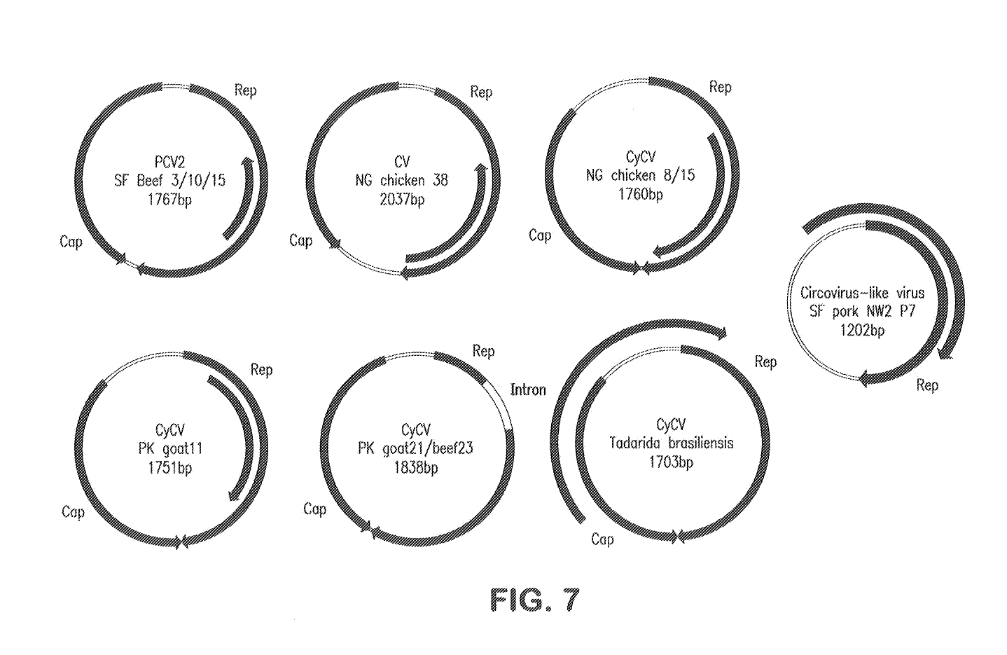

The genome of the chicken cycloviruses and circovirus has features characteristic of their genera including the absence and presence respectively of an intergenic region between the 3′ of both major ORFs (FIG. 7). CV-NG chicken 38 has the typical stem-loop structure and nonamer sequence of circoviruses (SEQ ID NO:60, 5′-TAGTATTAC-3′) while the nonamer sequence of chicken cycloviruses (SEQ ID NO:58, 5′-TAATACTAA-3′) slightly differed from those of cycloviruses in human and chimpanzee feces (SEQ ID NO:59, 5′-TAATACTAT-3′). No suitably located ATG is identified for the ORF encoding the Cap protein of CV-NG chicken 38. Considering the common use of alternative start codons in avian circoviruses, ATA is considered as the most likely start codon producing a Cap ORF of comparable size with that of CoC V. CoCV uses the alternative start codon ATA for both the Rep and Cap ORFs.

The putative Rep proteins of the chicken cycloviruses are 278 amino acids (aa) and from 47% to 74% similar to the Rep of previously reported cycloviruses (Table 5). The deduced Cap proteins are 222 aa long typical of cycloviruses (average 220 aa) exhibiting 14% to 48% similarity with those of reported cycloviruses. The chicken circovirus has a Rep protein of 317 aa, as does CoCV, with which it shared 93% amino acid identity (Table 5). The Cap protein of the chicken circovirus is 273 aa, also the same length as CoCV, with an amino acid similarity of 98%.

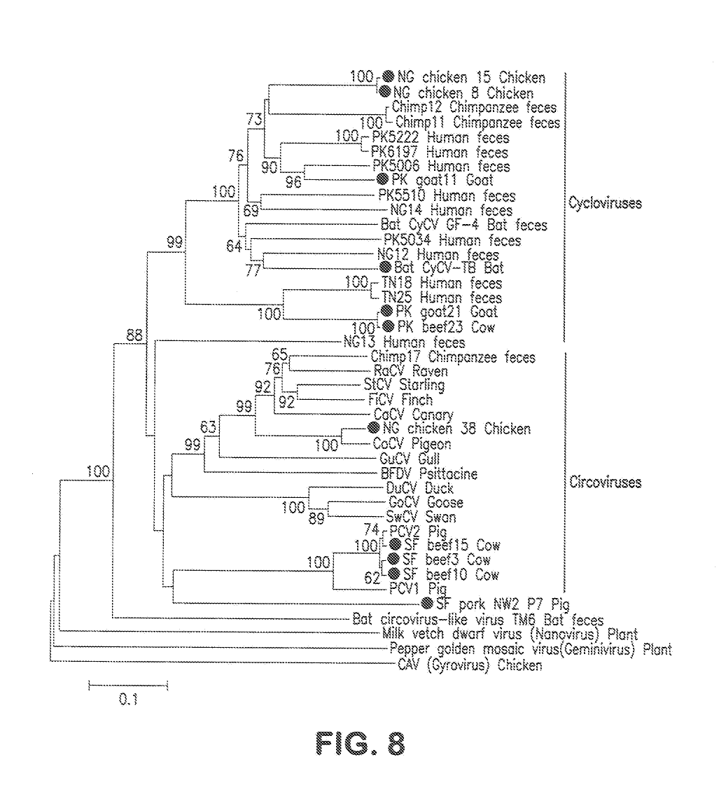

A phylogenetic analysis of the Rep confirmed that CyCV-NG chicken 8/15 clustered with cycloviruses while CV-NG chicken 38 is closely related to CoCV (FIG. 8).

### Example 9

### Cyclovirus and Circovirus Isolated from Bovids

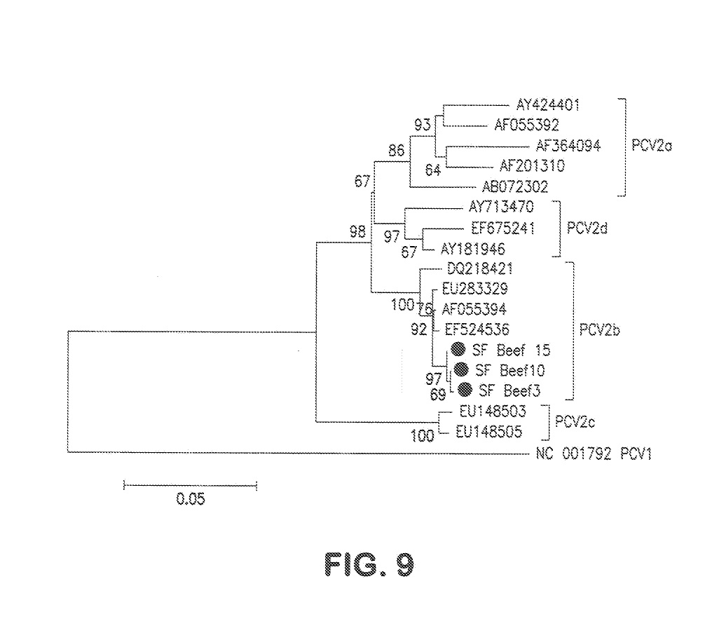

Circovirus-like Rep sequences were detected in 9 out of 19 beef sample from supermarkets in San Francisco, US. Five arere PCV2 sequences, and four are similar to the previously reported circovirus-like sequence NW2 with higher than 96% nucleotide identity over a Rep fragment of approximately 400 nucleotides amplified by degenerate PCR. There are 17/70 positive beef specimens, with 7 cyclovirus sequences, 5 PCV2 sequences and 5 circovirus-like sequences. The full-length genomes of PCV2 are obtained from 3 US beef specimens (PCV2 SF Beef3, 10 and 15). The 3 PCV2 from beef are 1767 nucleotides in length, differing at 5, 14, 15 nucleotides respectively with one another and sharing 99% nucleotide identity with PCV2 strains from pigs. Phylogenetically all clustered with the PCV2b genotypes (FIG. 9). One cyclovirus from beef is also sequenced (CyCV-PK beef23).

8 out of 73 goats and sheep specimens are PCR positive for Rep, all from the cyclovirus genus. Two cycloviruses genomes are obtained from Pakistani goats (CyCV-PK goat11, CyCV-PK goat21). The genomes of CyCV-PK goat21 and CyCV-PK beef23 are both 1838 nucleotides, shared 99% nucleotide identity, differing only at 2 nucleotides resulting in 1 aa difference in Rep (FIG. 7). The Rep gene of CyCV-PK goat21 and CyCV-PK beef23 are both interrupted by a 169 bp intron with typical splice donor (GT) and splice acceptor site (AG) as are the related Cy-CV TN18 and 25. Both Rep proteins are 280 aa, sharing 48% to 75% similarity with known cycloviruses (Table 5). The deduced Cap proteins are 212 aa, showing 14% to 57% similarity with those of known cycloviruses and having the highest similarity of 57% with Cy-CV TN18/25. The stem-loop structure contains slightly modified nonamer sequence (SEQ ID NO:67, 5′-TAATACTAG-3′) comparing with cycloviruses identified in human and chimpanzee feces (SEQ ID NO:59, 5′-TAATACTAT-3′). Phylogenetically, CyCV-PK goat21 and CyCV-PK beef23 cluster with human feces derived CyCV TN18 and TN25 (FIG. 8).

The CyCV-PK goat11 genome is 1751 nucleotides long encoding a 278 aa Rep and a 231 aa Cap protein (FIG. 7). Its nonanucleotide motif (SEQ ID NO:59, 5′-TAATACTAT-3′) is the same as the chimpanzee and human stool CyCV. The Rep of CyCV-PK goat11 shows 83% similarity with a human feces derived CyCV-PK5006, while the Cap shows <42% aa similarity with other cycloviruses (Table 5). Phylogenetically CyCV-PKgoat11 distantly clusters with other CyCV from human Pakistani feces (FIG. 8).

### Example 10

### Cyclovirus and Circovirus Isolated from Porcine

In total, 18 of the 22 pork products (muscle, ground pork and ham) from San Francisco supermarkets are PCR positive for circovirus-like Rep sequence. 12 samples are positive for PCV2 and 1 sample is PCV1. Six samples are closely related to one another and to the previously reported circovirus-like virus pork NW2 sequence. In one pork sample, both PCV2 and NW2 are detected. The full-length genomes of circovirus-like virus SF pork NW2 P7 is obtained as well as the partial genomes of closely related strains pork NW2 P8 (1050 bp) and NW2 P9 (899 bp). All share more than 97% nucleotide identity with each other.

Circovirus-like virus SF pork NW2 P7 genome is only 1202 nucleotides and circular, much shorter than known circoviruses and cycloviruses. Its genome organization resembles those of the single stranded circular DNA anelloviruses with two overlapping ORFs in the same direction (FIG. 7) but with a shorter genome. No suitably located ATG is identified for ORF1 encoding the putative Rep protein, but GTC is considered a possible alternative. The initiation codon for the putative ORF2 is ATG. The putative Rep protein of circovirus-like virus SF pork NW2 P7 is 221 aa, with 34% to 46% similarity to the Rep of known cycloviruses, and 39% to 46% similarity to circoviruses (Table 5). The ORF2 encodes a putative protein of 177 aa, which has low aa similarity (41%) with a hypothetical protein m169 of Muromegalovirus, a member of the Herpesviridae family. A stem-loop structure is found 51 nucleotides upstream of ORF2 with no homology to those of circoviruses or cycloviruses. Phylogenetic analyses of its circovirus-like Rep proteins shows that it fell outside the circovirus group but grouped together with the combined circoviruses and cyclovirus clades (FIG. 8).

### Example 11

### Cyclovirus and Circovirus Isolated from Bat

The pectoral muscle, digestive tract and fecal specimen from an individual male Brasilian free-tailed bat (Tadarida brasiliensis) in Texas, United States, in 2009 were all PCR positive for circovirus-like Rep sequence. The 3 sequences are identical with one another and belong to the cyclovirus Glade indicating that this virus infected this bat rather than simply consumed and excreted. The full-length genome of this cyclovirus is sequenced from muscle tissue, and tentatively named cyclovirus Tadarida brasiliensis (CyCV-TB).

The CyCV-TB genome is 1703 nucleotides, with a typical cyclovirus genome organization (FIG. 7). The putative Rep protein of CyCV-TB is 278 amino acids (aa), with 44% to 71% similarity to the Rep of known cycloviruses. CyCV-TB shows the highest aa similarity of 71% to CyCV NG12 from a Nigerian human feces, and 68% aa similarity with the CyCV-GF4 genome previously reported in bat guano from a Californian roost (Table 5). The deduced Cap protein is 225 aa, showing 12% to 48% similarity with those of cycloviruses found in human and chimpanzee, and 28% aa similarity with the bat guano derived CyCV-GF4. The highly conserved stem-loop structure with the nonamer sequence (SEQ ID NO:59, 5′-TAATACTAT-3′), identical to that in cycloviruses from human and chimpanzee feces, is present in the 5′-intergenic region.

The International Committee for the Taxonomy of Viruses suggested criteria for circovirus species demarcation as genome nucleotide identities of less than 75% and capsid protein amino acid identities of less than 70%. This example provides on circular single-stranded DNA viruses in the tissue of farm animals and a wild bat. Based on the distance criteria, CV-NG chicken 38 therefore appears to be a subtype of CoCV, and SF Beef3, 10 and 15 are strains of PCV2b. Four new species of cycloviruses are characterized, including CyCV-NG chicken 8/15, CyCV-PK goat11, CyCV-PK goat21/beef23, and CyCV-TB. Circovirus-like SF pork NW2 P7 genome is unusually small and only loosely related to circoviruses or cycloviruses and because of its unusual genome size and organization, its classification remains uncertain. The detection of apparently truncated circular DNA genome is reminiscent of that reported for a distantly related group of circular DNA viruses recently detected in chimpanzee stool and can reflect the presence of defective genome requiring trans-complementation by a helper virus.

Infection of different animal species by very closely related viruses includes PCV2 in pork and beef, CoCV in pigeon and chicken, CyCV-PK goat21/beef23 in goat and cow, and Circovirus-like virus SF pork NW2 in pork and beef Given that circoviruses have been estimated to have a mutation rate approaching those of RNA viruses, the presence of nearly identical viruses in different hosts may reflect recent cross-species viral transmissions.

TABLE 1
Sample collections tested by the degenerate primers for replication-associated proteins

| | | | | | No. of specimens (%) | | | | | | | |
|---|---|---|---|---|---|---|---|---|---|---|---|---|
| | | | | | | | | | | | With | |
| | Collection | | | | Pan-Rep | | | | | | other | |
| Sample | site(s) | Specimen | No. of | | PCR- | | With | | With | | Rep | |
| collection | (country) | type | Specimens | | positive | | cyclovirus | | circovirus | | proteins | |
| South Asian | Pakistan, | Human | 57 | (diseased) | 12 | (21.4) | 9 | (15.8) | 0 | (0.0) | 3 | (5.3) |
| children | Afghanistan | stool | 9 | (contact) | 3 | (33.3) | 3 | (33.3) | 0 | (0.0) | 0 | (0.0) |
| with AFP | | | 41 | (control) | 8 | (19.5) | 7 | (17.1) | 0 | (0.0) | 1 | (2.4) |
| and healthy | | | | | | | | | | | | |
| contact and | | | | | | | | | | | | |
| control | | | | | | | | | | | | |
| children | | | | | | | | | | | | |
| Minnesotans | United | Human | 140 | (diseased) | 7 | (5.0) | 0 | (0.0) | 7 | (5.0) | 0 | (0.0) |
| with | States | stool | 107 | (control) | 6 | (5.6) | 0 | (0.0) | 6 | (5.6) | 0 | (0.0) |
| gastroenteritis | | | | | | | | | | | | |
| and | | | | | | | | | | | | |
| healthy | | | | | | | | | | | | |
| controls | | | | | | | | | | | | |
| Nigerian | Nigeria | Human | 96 | | 18 | (18.8) | 9 | (9.4) | 2 | (2.1) | 7 | (7.3) |
| children | | stool | | | | | | | | | | |
| with AFP | | | | | | | | | | | | |
| Tunisian | Tunisia | Human | 96 | (diseased) | 7 | (7.3) | 7 | (7.3) | 0 | (0.0) | 0 | (0.0) |
| children | | stool | 96 | (contact) | 9 | (9.4) | 7 | (7.3) | 1 | (1.0) | 1 | (1.0) |

| | | | | | | | | | | | |
|---|---|---|---|---|---|---|---|---|---|---|---|
| with AFP | | | | | | | | | | | |
| and healthy | | | | | | | | | | | |
| contact | | | | | | | | | | | |
| children | | | | | | | | | | | |
| African | Middle | Chimpanzee | 44 | 9 | (20.5)* | 6 | (13.6) | 3 | (6.8) | 1 | (2.3) |
| chimpanzees | African | stool | | | | | | | | | |
| | countries* | | | | | | | | | | |
| U.S. blood | United | Human | 96 | 0 | (0.0) | 0 | (0.0) | 0 | (0.0) | 0 | (0.0) |
| donors | States | plasma | | | | | | | | | |
| African bush | Africa | Human | 113 | 0 | (0.0) | 0 | (0.0) | 0 | (0.0) | 0 | (0.0) |
| hunters | | plasma | | | | | | | | | |
| U.S. pork | United | Pork | 13 | 9 | (69.2) | 0 | (0.0) | 9 | (69.2) | 0 | (0.0) |
| products | States | | | | | | | | | | |
| Pakistani | Pakistan | Chicken | 13 | 0 | (0.0) | 0 | (0.0) | 0 | (0.0) | 0 | (0.0) |
| meat | | Beef | 26 | 5 | (19.2) | 4 | (15.4) | 1 | (3.8) | 0 | (0.0) |
| | | Goat | 18 | 7 | (38.9) | 7 | (38.9) | 0 | (0.0) | 0 | (0.0) |
| Nigerian meat | Nigeria | Chicken | 40 | 30 | (75.0) | 22 | (55.0) | 8 | (20.0) | 0 | (0.0) |
| | | Beef | 25 | 3 | (12.0) | 3 | (12.0) | 0 | (0.0) | 0 | (0.0) |
| | | Camel | 27 | 3 | (11.1) | 3 | (11.1) | 0 | (0.0) | 0 | (0.0) |
| | | Goat | 26 | 0 | (0.0) | 0 | (0.0) | 0 | (0.0) | 0 | (0.0) |
| | | Sheep | 29 | 1 | (3.4) | 1 | (3.4) | 0 | (0.0) | 0 | (0.0) |
| Total | | | 1,112 | 137 | | 88 | | 37 | | 13 | |

*Specimens are collected from Tanzania, Cameroon, Uganda, Rwands, Central Africa Republic, Republic of the Congo, and Democratic Republic of the Congo. In one chimp specimen, 2 different Rep sequences are obtained by subcloning

TABLE 2
Genome organization of newly discovered cycloviruses
and representative circoviruses*

| | | | | Length (nt) of region (start-end)* | |
|---|---|---|---|---|---|
| Virus type and | | No. of aa | | 5′ | 3′ |
| circular DNA | No. of nt | in protein | | Intergenic | Intergenic |
| virus species | in the genome | Rep | Cap | region | region |
| Cycloviruses | | | | | |
| PK5006 | 1,723 | 278 | 219 | 230(1516-22) | |
| PK5222 | 1,740 | 279 | 218 | 247(1516-22) | |
| PK5510 | 1,759 | 280 | 219 | 271(1679-190) | |
| FK5034 | 1,780 | 277 | 218 | 293(1691-203) | |
| PK6197 | 1,741 | 279 | 218 | 248(1516-22) | |
| Chimp11 | 1,750 | 280 | 220 | 258(1515-22) | |
| Chimp12 | 1,747 | 280 | 220 | 255(1515-22) | |
| NG12 | 1,794 | 281 | 218 | 284(1691-180) | 7(1027-1033) |
| NG14 | 1,795 | 286 | 230 | 245(1707-156) | |
| TN18 | 1,867 | 286 | 222 | 160(1762-54) | 6(1087-1092) |
| TN25 | 1,867 | 286 | 222 | 160(1762-54) | 6(1087-1092) |
| NG13 | 1,699 | 307 | 221 | 105(1622-27) | 4(952-955) |
| Circoviruses | | | | | |
| Chimp17 | 1,935 | 291 | 232 | 198(1772-34) | 162(911-1072) |
| MN614 | 1,767 | 314 | 233 | 83(1735-50) | 37(996-1032) |
| MN500 | 1,768 | 314 | 233 | 83(1736-50) | 38(996-1033) |
| PCV1 | 1,759 | 312 | 233 | 82(1724-46) | 36(986-1021) |
| PCV2 | 1,768 | 314 | 233 | 83(1736-50) | 38(996-1033) |
| DuCV | 1,991 | 292 | 257 | 110(1929-47) | 228(927-1154) |
| GoCV | 1,821 | 293 | 250 | 132(1762-72) | 54(955-1008) |
| CoCV | 2,037 | 317 | 273 | 90(1988-40) | 171(995-1165) |
| RaCV | 1,898 | 291 | 243 | 86(1848-35) | 204(912-1115) |
| SwCV | 1,785 | 293 | 251 | 107(1726-47) | 40(930-969) |
| BFDV | 1,993 | 299 | 244 | 126(1975-107) | 232(1008-1239) |
| GuCV | 2,035 | 305 | 245 | 207(1928-99) | 172(1018-1189) |
| FiCV | 1,962 | 291 | 249 | 29(1962-28) | 307(905-1211) |
| StCV | 2,063 | 289 | 276 | 79(2021-36) | 283(907-1189) |
| CaCV | 1,952 | 290 | 250 | 77(1907-31) | 249(905-1153) |

*15 genome sequences obtained by this study are shown in Bold. Nucleotide position 1 is set at the residue “A” at position 8 of the nonamer sequence; nt = nucleotides; aa = amino acids.

TABLE 3
Chimpanzee stool samples

| | | Collection Date | | |
|---|---|---|---|---|
| Sample ID | Collection Site | (mm/dd/yy) | Rep Screen | Rep ID |
| BB093 | Cameroon, Boumba Bek | 06/07/03 | Negative | |
| BQ399 | Cameroon, Belgique | 06/10/04 | Negative | |
| CP381 | Cameroon, Campo-Ma'an | 03/24/04 | Negative | |
| DG523 | Cameroon, Diang | 09/01/04 | Negative | |
| DP152 | Cameroon, Doumo Pierre | 11/13/03 | Negative | |
| DP271 | Cameroon, Douroo Pierre | 03/24/04 | Negative | |
| EK503 | Cameroon, E'kom | 07/11/04 | Negative | |
| LB313 | Cameroon, Lobéké | 01/10/04 | Negative | |
| MB615 | Cameroon, Mambele | 11/15/03 | Negative | |
| MF1290 | Cameroon, Mamfé | 08/21/05 | Negative | |
| MP1309 | Cameroon, Metep | 12/02/05 | Negative | |
| MT53 | Cameroon, Minta | 05/25/03 | Positive | Chimp53 |
| ME2525 | Central African Republic, | 07/19/07 | Negative | |
| | Melongodi | | | |
| ME2533 | Central African Republic, | 07/24/07 | Negative | |
| | Melongodi | | | |
| BA499 | Democratic Republic of the | 01/26/06 | Negative | |
| | Congo, Bafwaboli | | | |
| BF1541 | Democratic Republic of the | 03/07/07 | Positive | Chimp41 |
| | Congo, Bafwasende | | | |
| BD3 | Democratic Republic of the | 02/10/02 | Negative | |
| | Congo, Bondo-Bili | | | |
| BO1773 | Democratic Republic of the | 03/22/07 | Positive | Chimp73 |
| | Congo, Bongbola | | | |
| EP878 | Democratic Republic of the | 09/01/06 | Negative | |
| | Congo, Epulu | | | |
| KA1703 | Democratic Republic of the | 04/19/07 | Negative | |
| | Congo, Kabuka | | | |
| MA1919 | Democratic Republic of the | 06/24/07 | Negative | |
| | Congo, Maiko National Park | | | |
| MU720 | Democratic Republic of me | 03/20/06 | Negative | |
| | Congo, Mungbere | | | |
| OP1299 | Democratic Republic of the | 01/14/07 | Negative | |
| | Congo, Opienge | | | |
| UB1432 | Democratic Republic of the | 01/13/07 | Positive | Chimp32 |
| | Congo, Ubangi | | | |
| WL99 | Democratic Republic of the | 02/27/04 | Negative | |
| | Congo, Walengola | | | |
| WA513 | Democratic Republic of the | 03/17/06 | Positive | Chimp13 |
| | Congo, Wanie-Rukula | | | |
| GT306 | Republic of the Congo, | 09/30/04 | Negative | |
| | Goualougo Triangle | | | |
| GT615 | Republic of the Congo, | 04/19/05 | Negative | |
| | Goualougo Triangle | | | |
| NY17 | Rwanda, Nyungwe | 09/07/02 | Positive | Chimp17 |
| NY401 | Rwanda, Nyungwe | 02/05/04 | Negative | |
| MH60 | Tanzania, Mahale | 10/24/03 | Negative | |
| MH70 | Tanzania, Mahale | 11/03/03 | Negative | |
| KG9 | Uganda, Kyambura Gorge | 06/09/07 | Negative | |
| KG16 | Uganda, Kyambura Gorge | 06/12/07 | Positive | Chimp161/ |
| | | | | Chimp162 |
| KB30 | Uganda, Kibale | 03/16/03 | Negative | |
| KB36 | Uganda, Kibale | 03/17/03 | Negative | |
| GM415 | Tanzania, Gombe | 04/09/04 | Negative | |
| GM491 | Tanzania, Gombe | 06/22/04 | Negative | |
| GM841 | Tanzania, Gombe | 10/13/05 | Negative | |
| GM1062 | Tanzania, Gombe | 12/20/04 | Negative | |
| GM1199 | Tanzania, Gombe | 04/05/07 | Negative | |
| GM495 | Tanzania, Gombe | 05/03/04 | Positive | Chimp11 |
| GM488 | Tanzania, Gombe | 05/03/04 | Positive | Chimp12 |
| GM476 | Tanzania, Gombe | 03/16/04 | Negative | |

TABLE 4
Comparison of cycloviruses and circoviruses based on amino acid identities of the replicase and capsid
protein*

| | PK 5006 | PK 5034 | PK522 | PK5510 | Chimp12 | NG12 | NG14 | NG13 | TN25 | Chimp17 |
|---|---|---|---|---|---|---|---|---|---|---|
| PK 5006 | | 65.7 | 79.8 | 67.1 | 68.7 | 65.5 | 66.8 | 46.2 | 50.2 | 41.4 |
| PK 5034 | 28.9 | | 66.3 | 64.5 | 63.9 | 67.1 | 62.7 | 45.6 | 52.9 | 40.6 |
| PK5222 | 30.1 | 41.9 | | 67.1 | 71.8 | 63.5 | 66.8 | 46.5 | 52.5 | 43.8 |
| PK5510 | 32.7 | 35.5 | 42.3 | | 69.7 | 65.7 | 69.3 | 46.0 | 52.9 | 39.2 |
| Chimp12 | 35.0 | 45.8 | 38.8 | 34.0 | | 61.4 | 62.8 | 44.4 | 47.3 | 39.2 |
| NG12 | 55.6 | 29.9 | 30.1 | 35.7 | 33.5 | | 66.4 | 42.1 | 52.7 | 42.0 |
| NG14 | 31.0 | 38.4 | 31.6 | 36.5 | 34.3 | 33.5 | | 44.7 | 51.1 | 41.7 |
| NG13 | 12.7 | 14.6 | 15.6 | 13.7 | 14.6 | 11.2 | 14.9 | | 49.1 | 50.5 |
| TN25 | 25.8 | 28.0 | 25.4 | 29.6 | 29.6 | 28.1 | 26.5 | 13.7 | | 40.3 |
| Chimp17 | 11.7 | 12.8 | 11.5 | 11.4 | 12.8 | 10.4 | 10.2 | 14.0 | 15.9 | |
| PCV2 | 10.9 | 14.0 | 14.9 | 13.1 | 17.2 | 12.0 | 12.2 | 17.0 | 16.7 | 23.7 |
| PCV1 | 12.4 | 11.9 | 14.9 | 11.5 | 12.4 | 12.4 | 11.3 | 17.5 | 16.6 | 22.3 |
| BFDV | 14.8 | 16.7 | 19.1 | 17.7 | 15.2 | 14.4 | 13.8 | 18.8 | 17.7 | 44.9 |
| CaCV | 13.9 | 16.9 | 14.5 | 18.3 | 16.7 | 15.0 | 14.2 | 18.3 | 19.7 | 50.5 |
| CoCV | 15.2 | 15.3 | 13.8 | 16.7 | 15.6 | 16.1 | 15.3 | 15.0 | 15.7 | 57.9 |
| StCV | 15.4 | 16.9 | 16.4 | 17.3 | 16.7 | 15.0 | 13.2 | 16.1 | 17.3 | 75.0 |
| DUCV | 11.2 | 12.5 | 13.2 | 13.2 | 12.6 | 10.3 | 12.1 | 15.7 | 14.4 | 17.9 |
| GoCV | 11.7 | 13.2 | 16.2 | 13.7 | 12.6 | 12.7 | 10.7 | 15.9 | 13.0 | 18.0 |
| GuCV | 12.3 | 12.6 | 14.1 | 15.4 | 14.8 | 14.8 | 9.5 | 20.2 | 15.8 | 42.2 |
| FiCV | 13.8 | 15.8 | 16.3 | 20.0 | 18.5 | 17.2 | 16.3 | 18.8 | 15.2 | 50.8 |
| RaCV | 13.1 | 15.1 | 14.1 | 14.6 | 15.9 | 15.2 | 15.3 | 17.1 | 16.6 | 46.3 |
| SwCV | 10.7 | 15.1 | 16.6 | 13.6 | 13.5 | 9.8 | 10.2 | 17.8 | 12.9 | 20.2 |

| | PCV2 | PCV1 | BFDV | CaCV | CoCV | StCV | DuCV | GoCV | GuCV | FiCV | RaCV | SwCV |
|---|---|---|---|---|---|---|---|---|---|---|---|---|
| PK 5006 | 39.6 | 39.2 | 42.3 | 43.0 | 41.2 | 43.2 | 40.4 | 41.0 | 42.1 | 42.1 | 41.0 | 42.4 |
| PK 5034 | 40.4 | 39.7 | 39.2 | 41.5 | 41.0 | 42.1 | 39.4 | 39.3 | 41.7 | 41.4 | 42.1 | 39.3 |
| PK5222 | 41.2 | 40.1 | 43.2 | 43.6 | 42.5 | 43.8 | 41.6 | 42.6 | 42.3 | 42.6 | 43.8 | 42.2 |
| PK5510 | 39.0 | 39.0 | 40.9 | 41.3 | 39.5 | 43.0 | 38.3 | 38.9 | 40.8 | 41.9 | 40.4 | 40.4 |
| Chimp12 | 38.5 | 37.1 | 38.6 | 40.1 | 39.0 | 39.6 | 37.5 | 38.5 | 39.6 | 38.8 | 39.9 | 39.2 |
| NG12 | 39.9 | 39.5 | 41.8 | 41.0 | 43.7 | 42.8 | 39.9 | 39.8 | 41.6 | 42.4 | 41.3 | 39.4 |
| NG14 | 39.9 | 39.2 | 41.1 | 43.4 | 42.2 | 44.0 | 40.4 | 40.2 | 42.1 | 43.2 | 43.2 | 42.4 |
| NG13 | 44.9 | 44.7 | 48.8 | 49.3 | 51.9 | 51.2 | 44.2 | 46.2 | 50.3 | 52.2 | 49.5 | 46.6 |
| TN25 | 37.5 | 37.8 | 40.8 | 41.9 | 41.3 | 41.8 | 42.3 | 41.0 | 40.7 | 40.3 | 39.6 | 40.7 |
| Chimp17 | 42.8 | 43.0 | 61.4 | 74.8 | 73.5 | 79.2 | 50.5 | 49.1 | 60.0 | 79.0 | 79.7 | 48.1 |
| PCV2 | | 87.1 | 40.6 | 42.1 | 46.1 | 43.6 | 44.7 | 44.2 | 40.3 | 44.3 | 41.2 | 43.8 |
| PCV1 | 66.7 | | 41.7 | 44.1 | 45.8 | 44.3 | 44.7 | 45.2 | 41.4 | 44.3 | 42.3 | 44.9 |
| BFDV | 24.1 | 24.0 | | 59.2 | 59.5 | 60.4 | 47.6 | 44.8 | 54.4 | 61.7 | 57.6 | 45.5 |
| CaCV | 25.6 | 26.0 | 39.9 | | 69.0 | 76.0 | 47.2 | 46.5 | 65.4 | 75.5 | 76.6 | 46.2 |
| CoCV | 23.1 | 21.7 | 43.9 | 46.6 | | 73.4 | 51.0 | 48.6 | 58.4 | 72.9 | 73.2 | 49.0 |
| StCV | 26.1 | 26.1 | 45.6 | 52.3 | 55.3 | | 49.8 | 50.2 | 62.5 | 83.4 | 78.9 | 50.2 |
| DUCV | 26.5 | 26.3 | 20.4 | 21.6 | 23.1 | 21.3 | | 82.9 | 46.3 | 51.2 | 49.5 | 78.8 |
| GoCV | 26.3 | 27.6 | 18.9 | 19.6 | 19.3 | 19.2 | 48.0 | | 45.3 | 49.5 | 47.7 | 85.7 |
| GuCV | 27.1 | 25.6 | 49.8 | 44.2 | 43.5 | 42.2 | 17.7 | 18.8 | | 64.8 | 61.0 | 46.0 |
| FiCV | 24.0 | 23.4 | 41.2 | 65.0 | 47.0 | 48.9 | 19.7 | 19.0 | 45.3 | | 77.7 | 50.5 |
| RaCV | 25.2 | 24.3 | 39.4 | 75.6 | 44.1 | 48.7 | 21.7 | 18.8 | 44.7 | 60.3 | | 48.1 |
| SwCV | 27.1 | 27.0 | 18.8 | 19.5 | 20.2 | 20.1 | 49.4 | 71.6 | 17.9 | 18.5 | 19.6 | |

*All data above the diagonal showed amino acid identity (%) of Rep proteins, while all data below the diagonal compared amino acid identity (%) of Cap proteins. PK6197, Chimp 11 and TNI 8 are not shown as their genome had high identity with PK5222 (93%), Chimp12 (98%), and TN25 (97%), respectively. MN614 and MN500 are also excluded as they show 99%) nucleotide similarity to PCV2.

TABLE 5
Comparison of chicken cyclovirus and circovirus with representative circoviruses based on amino acid identities
of the replicase and capsid protein.

| | NG | NG | | | | | | | | | | | | |
|---|---|---|---|---|---|---|---|---|---|---|---|---|---|---|
| | chicken | Chicken | | | | | | | | | | | | |
| Virus | 8 | 38 | PCV2 | PCV1 | BFDV | CaCV | CoCV | StCV | DuCV | GoCV | GuCV | FiCV | RaCV | SwCV |
| NG | | 17.5 | 13.5 | 17.2 | 16.0 | 18.2 | 17.1 | 15.9 | 12.7 | 12.6 | 16.2 | 18.2 | 17.4 | 13.9 |
| Chicken 8 | | | | | | | | | | | | | | |
| NG | 42.9 | | 25.1 | 25.5 | 43.9 | 49.1 | 97.8 | 57.0 | 22.0 | 21.6 | 39.2 | 49.6 | 46.1 | 22.5 |
| Chicken 8 | | | | | | | | | | | | | | |
| PCV2 | 44.3 | 45.4 | | 66.7 | 27.4 | 27.5 | 24.7 | 27.9 | 24.8 | 24.1 | 28.1 | 26.2 | 26.8 | 23.5 |
| PCV1 | 43.2 | 44.9 | 87.1 | | 27.9 | 29.3 | 25.0 | 27.4 | 24.0 | 22.8 | 28.9 | 27.7 | 27.1 | 23.6 |
| BFDV | 41.1 | 60.9 | 40.5 | 41.6 | | 41.1 | 43.9 | 45.5 | 22.3 | 21.9 | 49.5 | 43.2 | 39.5 | 21.0 |
| CaCV | 44.1 | 70.3 | 41.7 | 44.3 | 58.8 | | 47.8 | 54.7 | 21.3 | 21.3 | 42.5 | 65.0 | 75.6 | 20.0 |
| CoCV | 43.2 | 93.4 | 45.1 | 45.2 | 58.5 | 68.6 | | 57.0 | 22.0 | 21.2 | 39.7 | 48.2 | 45.6 | 22.5 |
| StCV | 46.0 | 74.7 | 43.3 | 44.6 | 60.4 | 76.5 | 73.4 | | 20.2 | 20.6 | 40.5 | 51.3 | 51.1 | 19.7 |
| DuCV | 40.1 | 51.0 | 43.6 | 44.0 | 47.4 | 47.7 | 50.7 | 49.8 | | 48.0 | 19.9 | 18.2 | 21.9 | 49.4 |
| GoCV | 40.7 | 49.0 | 43.8 | 44.9 | 44.6 | 47.0 | 48.3 | 50.2 | 82.9 | | 24.5 | 21.1 | 22.3 | 71.6 |
| GuCV | 42.6 | 58.7 | 39.8 | 40.9 | 53.2 | 66.1 | 58.1 | 62.8 | 46.5 | 45.1 | | 44.1 | 43.2 | 23.9 |
| FiCV | 42.7 | 73.5 | 43.6 | 44.1 | 61.7 | 75.5 | 72.5 | 83.4 | 51.8 | 50.0 | 64.5 | | 60.3 | 21.1 |
| RaCV | 43.7 | 75.9 | 40.9 | 42.0 | 58.2 | 77.3 | 73.2 | 79.7 | 49.8 | 48.1 | 61.8 | 78.0 | | 22.3 |
| SwCV | 41.1 | 49.0 | 44.2 | 45.2 | 45.3 | 46.6 | 48.6 | 50.2 | 78.8 | 85.7 | 45.8 | 51.1 | 48.4 | |

Note:
all data below the diagonal showed amino acid identity (%) of Rep proteins, while all data above the diagonal compared amino acid identity (%) of Cap proteins. NG chicken 15 is not presented as it had 99% nucleotide similarity with NG chicken 8. Sequences for BFDV (AF071878), CaCV (AJ301633), CoCV (AF252610), DuCV (DQ100076), GoCV (AJ304456), GuCV (DQ845074), FiCV (DQ845075), RaCV (DQ146997), StCV (DQ172906), SwCV(EU056310), PCV1(AY660574), PCV2 (AY424401) are from GenBank.

Although the invention has been described with reference to the above example, it will be understood that modifications and variations are encompassed within the spirit and scope of the invention. Accordingly, the invention is limited only by the following claims.

What is claimed is:

1. An isolated nucleic acid molecule comprising a nucleotide sequence having at least 60% identity to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof.

2. An isolated nucleic acid molecule comprising a nucleotide sequence selected from the group consisting of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, and a complement thereof.

3. An isolated nucleic acid molecule comprising a nucleotide sequence that hybridizes under highly stringent conditions to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof.

4. An isolated nucleic acid molecule comprising a nucleotide sequence that hybridizes under highly stringent conditions to a nucleotide sequence encoding a protein selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, SEQ ID NO:30, SEQ ID NO:32, SEQ ID NO:33, SEQ ID NO:35, SEQ ID NO:36, SEQ ID NO:38, SEQ ID NO:39, SEQ ID NO:41, or a complement thereof.

5. The nucleic acid molecule of claim 1, wherein the nucleotide sequence is at least 80% identical to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof.

6. The nucleic acid molecule of claim 1, wherein the nucleotide sequence is at least 95% identical to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof.

7. The nucleic acid of claim 1, wherein the nucleotide sequence comprises an open reading frame encoding a protein selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, SEQ ID NO:30, SEQ ID NO:32, SEQ ID NO:33, SEQ ID NO:35, SEQ ID NO:36, SEQ ID NO:38, SEQ ID NO:39, SEQ ID NO:41, and conservative variants thereof.

8. A substantially purified protein comprising an amino acid sequence at least 60% identical to a sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, SEQ ID NO:30, SEQ ID NO:32, SEQ ID NO:33, SEQ ID NO:35, SEQ ID NO:36, SEQ ID NO:38, SEQ ID NO:39, SEQ ID NO:41.

9. The protein of claim 8, comprising a sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41.

10. A composition comprising a protein of claim 8.

11. A composition comprising a nucleic acid molecule of claim 1.

12. An isolated antibody that specifically binds to a protein of claim 8.

13. A purified serum comprising a polyclonal antibody that specifically binds to a protein of claim 15.

14. An isolated cyclovirus comprising a nucleic acid molecule of claim 1.

15. An expression vector comprising a nucleic acid molecule of claim 1.

16. A host cell comprising the expression vector of claim 15.

17. A method of detecting an cyclovirus nucleic acid comprising:
a) contacting a sample suspected of containing an cyclovirus nucleic acid with a nucleotide sequence that hybridizes under highly stringent conditions to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof; and
b) detecting the presence or absence of hybridization.

18. A method of detecting a cyclovirus nucleic acid comprising:
a) amplifying the nucleic acid of a sample suspected of containing cyclovirus nucleic acid with at least one primer that hybridizes to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof to produce an amplification product; and
b) detecting the presence of an amplification product, thereby detecting the presence of the cyclovirus nucleic acid.

19. A method of detecting a cyclovirus infection in a sample comprising:
a) contacting a sample suspected of containing a cyclovirus protein with an antibody that specifically binds to an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41, to form a protein/antibody complex; and
b) detecting the presence of the protein/antibody complex, thereby detecting the presence of the cyclovirus protein.

20. A kit for detecting a cyclovirus nucleic acid comprising at least one nucleic acid molecule that hybridizes under highly stringent conditions to a nucleic acid molecule of claim 1.

21. A kit for detecting a cyclovirus nucleic acid comprising at least one oligonucleotide primer that hybridizes to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof, under highly stringent PCR conditions.

22. A method of assaying for an anti-cyclovirus compound comprising:
a) contacting a sample containing a cyclovirus with a test compound, the cyclovirus comprising a genome that hybridizes under highly stringent conditions to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof; and
b) determining whether the test compound inhibits cyclovirus replication, wherein inhibition of cyclovirus replication indicates that the test compound is an anti-cyclovirus compound.

23. A method of treating or preventing a cyclovirus infection in a subject comprising; administering to the subject an antigen encoded by a cyclovirus, the cyclovirus comprising a genome that hybridizes under highly stringent conditions to a nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof; thereby treating or prevention infection in the subject.

24. A method of treating or preventing a cyclovirus infection in a subject comprising: administering to the subject an antigen encoded by a cyclovirus, wherein the antigen comprises an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41, thereby treating or prevention infection in the subject.

25. A vaccine for the prevention of gastrointestinal tract, respiratory, nervous system or blood infection in a subject, comprising: a cyclovirus or at least one cyclovirus antigen from the cyclovirus which induces a gastrointestinal tract, respiratory, nervous system or blood infection in a subject and a pharmacologically acceptable carrier wherein the cyclovirus has gastrointestinal tract, respiratory, nervous system or blood infection inducing characteristics.

26. The vaccine of claim 25, wherein the cyclovirus antigen has an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID NO:23, SEQ ID NO:24, SEQ ID NO:26, SEQ ID NO:27, SEQ ID NO:29, and SEQ ID NO:30, SEQ ID NO:32, SEQ ID NO:33, SEQ ID NO:35, SEQ ID NO:36, SEQ ID NO:38, SEQ ID NO:39, SEQ ID NO:41.

27. A method for detecting and serotyping cyclovirus in a sample comprising:
a) contacting a first portion of the sample with a first pair of primers in a first amplification protocol, wherein the first pair of primers have an associated first characteristic amplification product if a cyclovirus is present in the sample;
b) determining whether or not the first characteristic amplification product is present;
c) contacting a second portion of the sample with a second pair of primers in a second amplification protocol, wherein the second pair of primers have an associated second characteristic amplification product if a cyclovirus is present in the sample and wherein the second pair of primers are different from the first pair of primers;
d) determining whether or not the second characteristic amplification product is present;
e) based on whether or not the first and second characteristic amplification product are present, selecting one or more subsequent pair of primers and contacting the one or more subsequent pair of primers with additional portions of the sample in subsequent amplification protocols, wherein each subsequent pair of primers is different from each pair of primers already used and wherein each subsequent pair of primers has an associated subsequent characteristic amplification product if a cyclovirus is present in the sample;
f) determining whether or not the associated characteristic amplification product for each subsequent pair of primers used is present;
g) repeating steps e) and for one or more subsequent pairs of primers if the cyclovirus cannot be serotyped based on the determinations of steps b), d), and f) until the cyclovirus can be serotyped, wherein the one or more subsequent pairs of primers are different from all pairs of primers used in earlier amplification protocols; and
h) determining the serotype or groups of serotypes of the cyclovirus that may be present in the sample.

28. The method of claim 27, wherein the cyclovirus has a genome comprising a nucleic acid sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof.

29. A method for detecting the presence of a cyclovirus in a sample comprising:
a) purifying RNA contained in the sample;
b) reverse transcribing the RNA with primers effective to reverse transcribe cyclovirus RNA to provide a cDNA;
c) contacting at least a portion of the cDNA with (i) a composition that promotes amplification of a nucleic acid and (ii) an oligonucleotide mixture wherein the mixture comprises at least one oligonucleotide that hybridizes to a highly conserved sequence of the sense strand of a cyclovirus nucleic acid and at least one oligonucleotide that hybridizes to a highly conserved sequence of the antisense strand of a cyclovirus nucleic acid;
d) carrying out an amplification procedure on the amplification mixture such that, if a cyclovirus is present in the sample, a cyclovirus amplicon is produced whose sequence comprises a nucleotide sequence of at least a portion of the cyclovirus genome; and
e) detecting whether an amplicon is present; wherein the presence of the amplicon indicates that a cyclovirus is present in the sample.

30. The method of claim 29, wherein the cyclovirus has a genome comprising a nucleic acid sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, or a complement thereof.

31. A vaccine for protecting an animal from infection by a cyclovirus, wherein the vaccine is selected from the group consisting of:
a) a genetically modified cyclovirus encoded by the isolated polynucleotide molecule according to claim 1; and
b) a viral vector comprising the isolated polynucleotide molecule according to claim 1;
wherein the vaccine is in an amount effective to produce immunoprotection against infection by a cyclovirus and the vaccine comprises a vaccine carrier acceptable for human or veterinary use.

32. A vaccine for the prevention of a systemic disease, respiratory disease complex, enteric disease, postweaning multisystemic wasting syndrome, porcine dermatitis and nephropathy syndrome or reproductive disorders in porcine, comprising:
a cyclovirus or at least one cyclovirus antigen from the cyclovirus which induces a systemic disease, respiratory disease complex, enteric disease, porcine dermatitis and nephropathy syndrome or reproductive disorders in porcine and a pharmacologically acceptable carrier wherein the cyclovirus has systemic disease, respiratory disease complex, enteric disease, postweaning multisystemic wasting syndrome, porcine dermatitis and nephropathy syndrome or reproductive disorders inducing characteristics.
